(19) United States
(12) Patent Application Publication
(10) Pub. No.: US 2016/0192963 A1
(43) Pub. Date: Jul. 7, 2016

(54) LIVING BODY INSERTION TOOL AND SUCTION METHOD

(71) Applicant: TERUMO KABUSHIKI KAISHA, Tokyo (JP)

(72) Inventors: Masakatsu KAWAURA, Sunnyvale, CA (US); Nao MANDAI, Mountain View, CA (US)

(73) Assignee: TERUMO KABUSHIKI KAISHA, Tokyo (JP)

(21) Appl. No.: 15/071,933

(22) Filed: Mar. 16, 2016

Related U.S. Application Data
(63) Continuation-in-part of application No. PCT/JP2014/073573, filed on Sep. 5, 2014.

(30) Foreign Application Priority Data
Sep. 21, 2013 (JP) 2013-196286

(51) Int. Cl. A61B 17/42; A61B 17/02

(52) U.S. Cl. CPC A61B 17/42; A61B 17/0218; A61B 2017/0042

(57) ABSTRACT

A living body insertion tool for being inserted into a body lumen includes an attachment face configured to attach to a living body tissue, a suction portion configured to suck the living body tissue to the attachment face side in a state in which the attachment face attaches to the living body tissue, and a blocking portion configured to block the living body tissue from reaching the suction portion. A suction method for inserting the living body insertion tool into the body lumen to suck the living body tissue to the living body insertion tool side includes sucking the living body tissue to a recessed portion of the attachment face such that the living body tissue does not attach to the suction portion.

### CROSS-REFERENCES TO RELATED APPLICATIONS

This application is a continuation of International Application No. PCT/JP2014/073573 filed on Sep. 5, 2014, and claims priority to Japanese Application No. 2013-196286 filed on Sep. 21, 2013, the entire content of both of which is incorporated herein by reference.

### TECHNICAL FIELD

The present disclosure relates to a living body insertion tool and a suction method.

### BACKGROUND DISCUSSION

In a patient suffering from urinary incontinence, particularly, for example, stress urinary incontinence, urine leakage can occur due to an abdominal pressure exerted during a normal exercise or by, for example, laughing, coughing, or sneezing. This can be attributable, for example, to loosening of the pelvic floor muscle, which is a muscle for supporting the urethra, caused by childbirth.

Surgical therapy is effective for treatment of urinary incontinence, and, for example, a belt-shaped implant called “sling” is used and placed indwelling in the body such that the urethra is supported by the sling (for example, refer to Japanese Patent Laid-Open No. 2010-99499). In order to put a sling indwelling inside the body, an operator would incise the vagina with a surgical knife to dissect the living body tissue between the urethra and the vagina, and then, using a puncture needle, make the dissected region and the outside communicate with each other through an obturator foramen. In such a state as just described, the sling is placed indwelling in the body.

Further, when the living body tissue between the urethra and the vagina is to be dissected, for example, a suction tool having a great number of suction ports is sometimes inserted into the vagina to suck the vaginal wall. By the suction, the living body tissue is expanded in a thicknesswise direction thereof and is placed into a state in which it can be dissected readily.

However, since the suction ports are very small, if they are covered directly with the living body tissue, then there is the possibility that suction force may be degraded remarkably, resulting in failure to sufficiently expand the living body tissue.

### SUMMARY

A living body insertion tool and a suction method are disclosed by which degradation of suction force can be prevented with relative certainty.

A living body insertion tool is disclosed for being inserted into a body lumen, including an attachment face configured to attach to a living body tissue, a suction portion configured to suck the living body tissue to the attachment face side in a state in which the attachment face attaches to the living body tissue, and a blocking portion configured to block the living body tissue from reaching the suction portion.

In accordance with an exemplary embodiment, the living body insertion tool is configured such that a recessed portion is formed on the attachment face, the suction portion is provided at a bottom portion of the recessed portion, and the blocking portion is configured from a plurality of linear members at least part of which is provided at the bottom portion and which individually have a linear shape or a shape of a belt.

The blocking portion may have a form of a grating formed by crossing the plurality of linear members with each other.

The blocking portion and the bottom portion may have a gap therebetween and be communicated with each other by the gap.

At least part of the linear members may be provided so as to connect side walls of the recessed portion to each other.

The linear members may be disposed so as to overlap with a suction direction of the suction portion.

The living body insertion tool may be configured such that the body lumen is a vaginal cavity and the living body insertion tool is inserted into and used with the vaginal cavity.

In another aspect, a suction method is disclosed for inserting a living body insertion tool into a body lumen to suck a living body tissue to the living body insertion tool side, the living body insertion tool including an attachment face configured to attach to the living body tissue, a suction portion configured to suck the living body tissue to the attachment face side in a state in which the attachment face attaches to the living body tissue, and a blocking portion configured to block the living body tissue from reaching the suction portion, the suction method including sucking the living body tissue to a recessed portion of the attachment face such that the living body tissue does not attach to the suction portion.

Suction force to a living body tissue which defines a body lumen varies in proportion to the area (hereinafter referred to as “suction area”) over which the living body tissue is sucked actually. Here, the suction area where the living body tissue directly contacts with a suction port to close the suction port and the suction area where the living body tissue is spaced apart from the suction port and the suction port is not closed are compared with each other. The former suction area can be smaller than the latter suction area. Therefore, the suction force to the living body tissue in the former suction area is lower than that in the latter suction area.

Therefore, according to the present disclosure, even in a state in which a living body tissue is sucked, a blocking portion can prevent the living body tissue from closing up a suction port with certainty. Consequently, a drop of suction force can be prevented with relative certainty.

### BRIEF DESCRIPTION OF THE DRAWINGS

FIG. 1 is a perspective view depicting a puncture apparatus to which a living body insertion device (first embodiment) is applied;

FIG. 2 is a lateral view of the puncture apparatus depicted in FIG. 1;

FIG. 3 is a plan view depicting an operation member possessed by the puncture apparatus depicted in FIG. 1;

FIGS. 4A and 4B depict a puncture member possessed by the puncture apparatus depicted in FIG. 1, wherein FIG. 4A is a perspective view and FIG. 4B is a sectional view taken along line IVB-IVB of FIG. 4A;

FIG. 5 is a sectional view of the puncture member depicted in FIG. 4A;

FIGS. 6A to 6C depict a state maintaining mechanism possessed by the puncture member depicted in FIG. 4A, wherein FIG. 6A is a top plan view and FIGS. 6B and 6C are sectional views;

FIGS. 7A to 7C are partial enlarged views depicting the state maintaining mechanism possessed by the puncture member depicted in FIGS. 4A and 4B, wherein FIGS. 7A and 7B are top plan views depicting modifications and FIG. 7C is a top plan view depicting the present embodiment;

FIGS. 8A and 8B depict a second anchor possessed by the puncture apparatus depicted in FIG. 1, wherein FIG. 8A is a sectional view and FIG. 8B is a sectional view depicting an engagement state with the puncture member;

FIGS. 9A and 9B depict a first anchor possessed by the puncture apparatus depicted in FIG. 1, wherein FIG. 9A is a sectional view and FIG. 9B is a sectional view depicting an engagement state with the puncture member;

FIG. 10 is a sectional view depicting a guide portion of a frame provided in the puncture apparatus depicted in FIG. 1;

FIG. 11 is a sectional view depicting the guide portion of the frame provided in the puncture apparatus depicted in FIG. 1;

FIG. 12 is a sectional view depicting the guide portion of the frame provided in the puncture apparatus depicted in FIG. 1;

FIG. 13 is a plan view depicting a fixation portion of the frame provided in the puncture apparatus depicted in FIG. 1;

FIG. 14 is a lateral view of an insertion tool possessed by the puncture apparatus depicted in FIG. 1;

FIGS. 15A and 15B depict a positional relationship between the puncture member and an obturator foramen (pelvis), wherein FIG. 15A is a lateral view and 15B is a front elevational view;

FIG. 16 is a partial enlarged view of a vaginal insertion member possessed by the insertion tool depicted in FIG. 14;

FIG. 17A is a sectional view depicting an example of the shape of a vaginal wall, and FIG. 17B is a sectional view depicting a state in which a vaginal insertion portion is inserted in the vagina depicted in FIG. 17A;

FIG. 18 is a view depicting an implant used together with the puncture apparatus depicted in FIG. 1;

FIG. 19 is a view illustrating an operation procedure of the puncture apparatus depicted in FIG. 1;

FIGS. 20A and 20B illustrate the operation procedure of the puncture apparatus depicted in FIG. 1, wherein FIG. 20A depicts the operation procedure when the operation is performed appropriately and FIG. 20B depicts the operation procedure when the operation is performed inappropriately;

FIG. 21 is a view depicting the operation procedure of the puncture apparatus depicted in FIG. 1;

FIGS. 22A and 22B are views illustrating the operation procedure of the puncture apparatus depicted in FIG. 1;

FIG. 23 is a lateral view depicting a relationship between the puncture apparatus and the pelvis when the puncture apparatus is in the state depicted in FIG. 22A;

FIGS. 24A and 24B are views illustrating the operation procedure of the puncture apparatus depicted in FIG. 1;

FIG. 25 is a lateral view depicting a relationship between the puncture apparatus and the pelvis when the puncture apparatus is in the state depicted in FIG. 24A;

FIG. 26 is a sectional view depicting a posture of the puncture member with respect to the urethra when the puncture apparatus is in the state depicted in FIG. 24B;

FIGS. 27A and 27B are views illustrating the operation procedure of the puncture apparatus depicted in FIG. 1;

FIGS. 28A and 28B are views illustrating the operation procedure of the puncture apparatus depicted in FIG. 1;

FIGS. 29A and 29B are lateral views illustrating an operation method of a vaginal insertion member possessed by a living body insertion device (second embodiment);

FIG. 30 is a lateral view of a vaginal insertion member possessed by a living body insertion device (third embodiment);

FIG. 31 is a view illustrating an operation procedure of a living body insertion device (fourth embodiment);

FIG. 32 is a view illustrating the operation procedure of the living body insertion device (fourth embodiment);

FIGS. 33A and 33B illustrate the operation procedure of the living body insertion device (fourth embodiment), wherein FIG. 33A is a view when the operation is performed appropriately and FIG. 33B is a view when the operation is performed inappropriately;

FIG. 34 is a lateral view of a urethral insertion member possessed by a living body insertion device (fifth embodiment);

FIG. 35 is a sectional view taken along line XXXV-XXXV of FIG. 34;

FIG. 36 is a transverse sectional view of a urethral insertion member possessed by a living body insertion device (sixth embodiment);

FIG. 37 is a lateral view of a urethral insertion member possessed by a living body insertion device (seventh embodiment);

FIG. 38 is a sectional view taken along line XXXVIII-XXXVIII of FIG. 37;

FIG. 39 is a plan view of a vaginal insertion member possessed by a living body insertion device (eighth embodiment);

FIG. 40 is a sectional view taken along line XL-XL of FIG. 39;

FIG. 41 is a plan view of a vaginal insertion member possessed by a living body insertion device (ninth embodiment);

FIG. 42 is a sectional view taken along line XLII-XLII of FIG. 41;

FIG. 43 is a longitudinal sectional view of a vaginal insertion member possessed by a living body insertion device (tenth embodiment);

FIG. 44 is a plan view of a vaginal insertion member possessed by a living body insertion device (eleventh embodiment);

FIG. 45 is a view illustrating an operation procedure of the vaginal insertion member depicted in FIG. 44;

FIG. 46 is a view illustrating the operation procedure of the vaginal insertion member depicted in FIG. 44;

FIG. 47 is a plan view of a vaginal insertion member possessed by a living body insertion device (twelfth embodiment);

FIG. 48 is a longitudinal sectional view of the vaginal insertion member depicted in FIG. 47;

FIG. 49 is a plan view of a vaginal insertion member possessed by a living body insertion device (thirteenth embodiment);

FIG. 50 is a sectional view taken along line L-L of FIG. 49;

FIG. 51 is a plan view of a vaginal insertion member possessed by a living body insertion device (fourteenth embodiment);

FIG. 52 is a sectional view taken along line LII-LII of FIG. 51;

FIG. 53 is an enlarged view of a protrusion of a vaginal insertion member possessed by a living body insertion device (fifteenth embodiment);

FIG. 54 is an enlarged view of a protrusion of a vaginal insertion member possessed by a living body insertion device (sixteenth embodiment);

FIG. 55 is a view illustrating an operation procedure of a living body insertion device (seventeenth embodiment);

FIG. 56 is a view illustrating the operation procedure of the living body insertion device (seventeenth embodiment);

FIG. 57 is a view illustrating an operation procedure of a living body insertion device (eighteenth embodiment);

FIG. 58 is a view illustrating the operation procedure of the living body insertion device (eighteenth embodiment);

FIG. 59 is an enlarged sectional view of a region [H] surrounded by a two-dot chain line in FIG. 57;

FIG. 60 is an enlarged sectional view of a region [I] surrounded by a two-dot chain line in FIG. 58;

FIG. 61 is a view illustrating an operation procedure of a living body insertion device (nineteenth embodiment);

FIG. 62 is a view illustrating the operation procedure of the living body insertion device (nineteenth embodiment);

FIG. 63 is a view illustrating an operation procedure of a living body insertion device (twentieth embodiment);

FIG. 64 is a view illustrating the operation procedure of the living body insertion device (twentieth embodiment);

FIG. 65 is a front elevational view of a support unit of the living body insertion device (twentieth embodiment);

FIG. 66 is a front elevational view of a support unit of a living body insertion device (twenty-first embodiment);

FIG. 67 is a front elevational view of a support unit of a living body insertion device (twenty-second embodiment);

FIG. 68 is a front elevational view of a support unit of a living body insertion device (twenty-third embodiment);

FIG. 69 is a front elevational view of a support unit of a living body insertion device (twenty-fourth embodiment);

FIG. 70 is a view of the support unit of FIG. 69 as viewed in a direction indicated by an arrow mark J;

FIG. 71 is a lateral view of a living body insertion device (twenty-fifth embodiment);

FIG. 72 is a lateral view of a living body insertion device (twenty-sixth embodiment); and

FIG. 73 is a lateral view of a living body insertion device (twenty-seventh embodiment).

### DETAILED DESCRIPTION

In the following, a living body insertion tool and a suction method of the present disclosure are described in detail in connection with preferred embodiments depicted in the accompanying drawings.

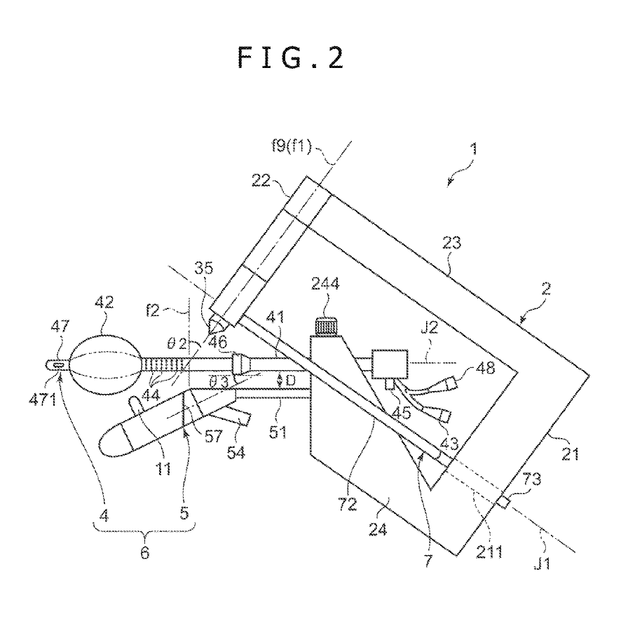

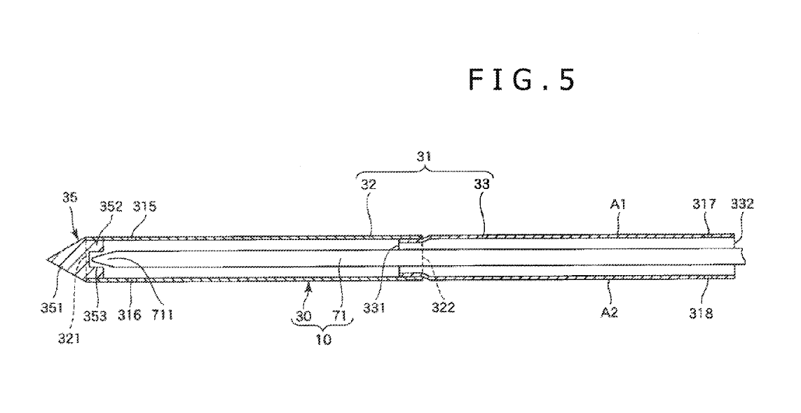

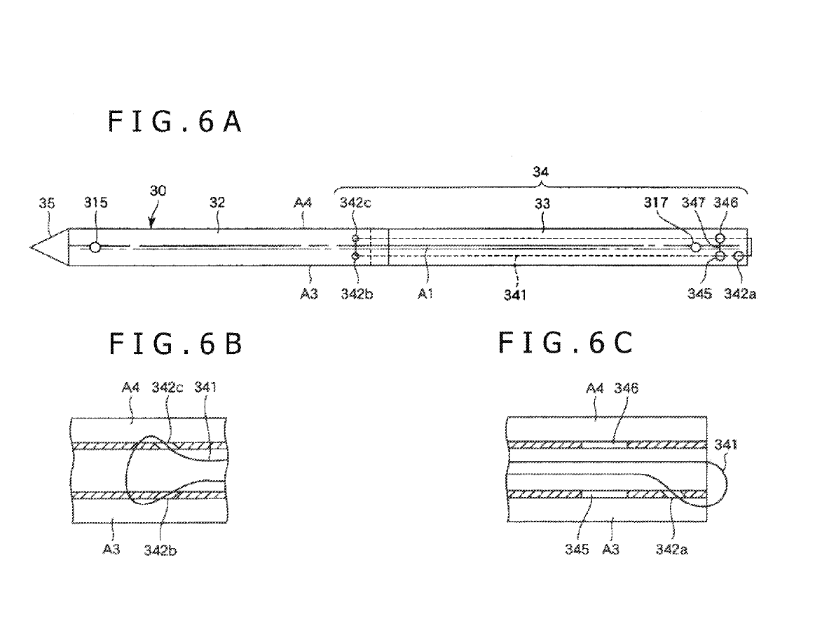

Note that, in the following description, the left side in FIG. 2 is referred to as “distal (end),” the right side as “proximal (end),” the upper side as “upper (side),” and the lower side as “lower (side)” for the convenience of description. FIG. 2 depicts a puncture apparatus (insertion tool) in a state in which the puncture apparatus is not used as yet, and in the following description, this state is referred to also as “initial state” for the convenience of description. Another state in which the puncture apparatus (insertion tool) depicted in FIG. 2 is mounted on a patient is referred to also as “mounted state.” Furthermore, in FIG. 5 and FIGS. 6A to 6C, a puncture member which extends in an arc is depicted in a linearly stretched state for the convenience of illustration.

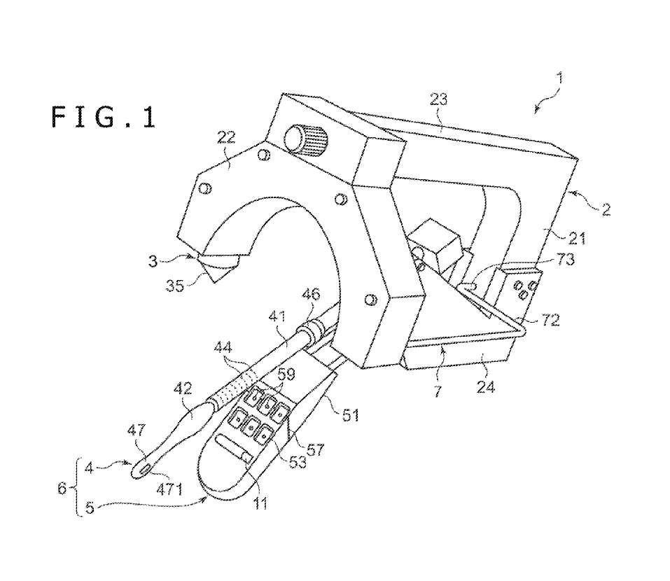

The puncture apparatus 1 depicted in FIGS. 1 and 2 can be used in treatment of urinary incontinence of women, for example, used to implant a living body tissue-supporting indwelling article for treatment of urinary incontinence of women into a living body.

The puncture apparatus 1 can include a frame (support member) 2, a puncture member (puncture needle) 3, a urethral insertion member (urethral insertion tool) 4, a vaginal insertion member (vaginal insertion tool) 5, an operation member 7, and anchors 81 and 82. The puncture member 3, urethral insertion member 4, vaginal insertion member 5, operation member 7 and anchors 81 and 82 are each supported on the frame 2. Further, in the puncture apparatus 1, the urethral insertion member 4 and the vaginal insertion member 5 configure an insertion tool (living body insertion device) 6. In the following, the components mentioned are described in order.

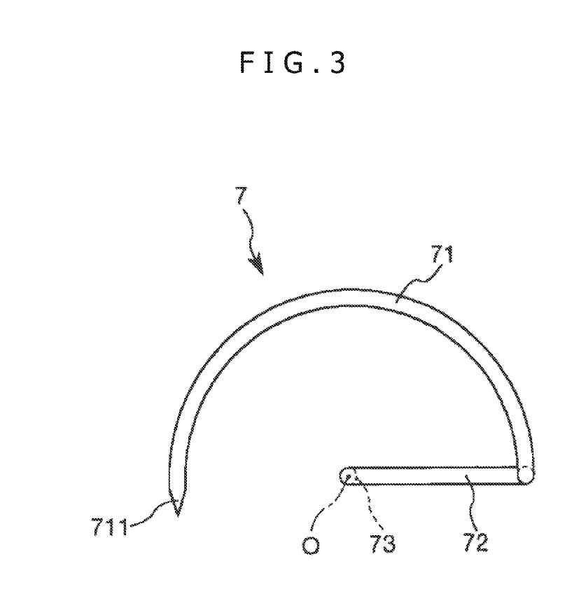

In accordance with an exemplary embodiment, the operation member 7 can be used to operate the puncture member 3. As depicted in FIGS. 1 to 3, such an operation member 7 as just described can include an insertion portion 71, a shaft portion 73, and an interlock portion 72 which interlocks the insertion portion 71 and the shaft portion 73 to each other. The insertion portion 71, interlock portion 72 and shaft portion 73 may be formed integrally with one another, or, alternatively, at least one of them may be formed as a separate member from the others of them.

The insertion portion 71 is configured to be inserted into the puncture member 3 and functions as a stylet for reinforcing the puncture member 3 from the inner side. By inserting the insertion portion 71 into the puncture member 3, the puncture member 3 is connected to the operation member 7, whereby an operation of the puncture member 3 by the operation member 7 is permitted. Such an insertion portion 71 as just described has an arcuate shape corresponding to the shape of the puncture member 3. The center angle of the insertion portion 71 is set in accordance with the center angle of the puncture member 3. A distal end portion 711 of the insertion portion 71 is tapering. Since the insertion portion 71 has the tapering distal end portion 711, the insertion of the insertion portion 71 into the puncture member 3 can be performed relatively smoothly.

The shaft portion 73 extends along an axis J1 which intersects with the center O of the insertion portion 71 and orthogonally intersects with a plane f1 which can include the insertion portion 71.

The interlock portion 72 interlocks a proximal end portion of the insertion portion 71 and a distal end portion of the shaft portion 73 to each other. Further, the interlock portion 72 has a substantially L shape bent at a substantially right angle at an intermediate portion thereof. The interlock portion 72 can function also as a grasping portion to be grasped by an operator when the operation member 7 is to be operated.

The operation member 7 having such a configuration as described above is configured so as to have a rigidity higher than that of the puncture member 3 (main body 31). The material constituting the operation member 7 is not limited particularly and, for example, various metal materials such as stainless steel, aluminum or aluminum alloys, titanium or titanium alloys and so forth can be used.

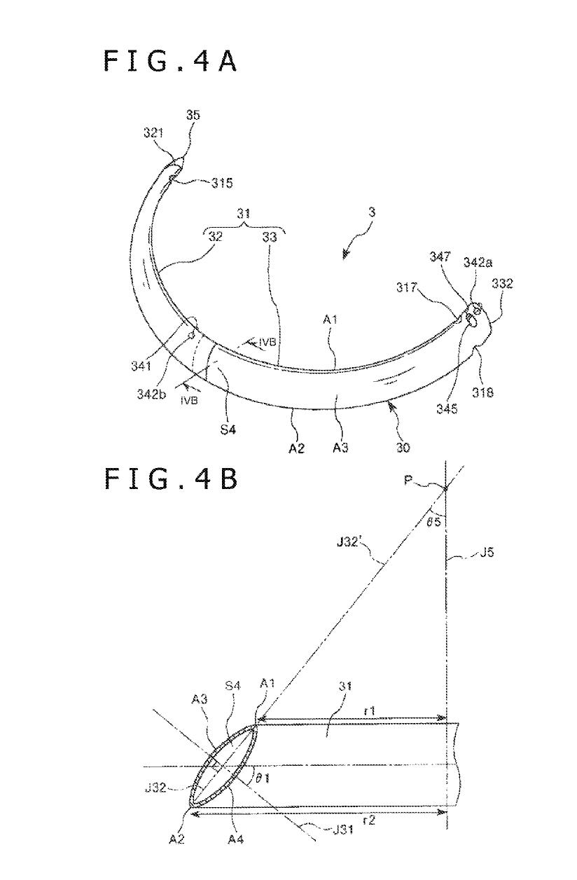

In accordance with an exemplary embodiment, the puncture member 3 can be used to puncture a living body. As depicted in FIG. 4A, the puncture member 3 can include an elongate sheath (medical tube) 30, and a needle body 35 provided at the distal end of the sheath 30. The sheath 30 can include a tubular main body 31 and a state maintaining mechanism 34.

The main body 31 is configured from an elongate pipe member (tube) and is open at the distal end and the proximal end thereof. Such a main body 31 as just described has an internal space into which an implant main body 91 can be inserted. Further, the main body 31 has a curved shape in which it is curved in an arc and has a flattened transverse sectional shape as depicted in FIG. 4B. Particularly, for example, the transverse sectional shape of the main body 31 at a central portion S4 in the longitudinal direction has a flattened shape having a minor axis J31 and a major axis J32. As hereinafter described, the implant main body 91 is disposed in the main body 31. Therefore, by forming the main body 31 in a flattened shape, the implant main body 91 can be controlled to a desired posture in the main body 31.

Meanwhile, the width (length in the major axis J32 direction) of the internal space of the main body 31 can be designed so as to be substantially equal to the width of a main body portion 911 hereinafter described of the implant main body 91. By the configuration, even if the implant main body 91 is moved, the frictional resistance between the implant main body 91 and the main body 31 can be reduced and unnecessary force is not applied to the implant main body 91. Further, the main body portion 911 can be disposed in the main body 31 in a state in which it can be sufficiently developed. However, the width (length in the direction of the major axis J32) of the internal space of the main body 31 may be smaller than the width of the main body portion 911. Since the width of the main body 31 is thereby suppressed, the puncture member 3 becomes less invasive.

Note that the flattened shape of the main body 31 is not limited particularly and can be made, for example, an elliptical shape, a sectional shape of a convex lens, a diamond shape rounded at the corners thereof, a rectangle (flattened shape) rounded at the corners thereof or a spindle shape having a width greater (diameter increased) at a central portion than at the opposite end portions thereof.

In the following description, for the convenience of description, an end portion positioned on the inner side (at one end portion) in the major axis J32 direction is referred to also as “inner circumferential portion A1,” an end portion positioned on the outer side (at the other end portion) in the major axis J32 direction is referred to also as “outer circumferential portion A2,” a face directed to the upper side is referred to also as “front face A3,” and a face directed to the lower side is referred to also as “back face A4” as depicted in FIG. 4B.

Where, as depicted in FIG. 4B, a plane including both of the center point of the arc of the central portion S4 and the center point of a transverse sectional shape across the longitudinal direction of the main body 31 (plane including the center axis of the main body 31) is represented as plane f9 and the angle between the plane f9 and the minor axis J31 at the central portion S4 is represented as inclination angle θ1, the inclination angle θ1 is preferably, for example, an acute angle. Where the inclination angle θ1 is an acute angle, an implant 9 hereinafter described can be disposed substantially in parallel to the urethra (urethral lumen) 1300 which is one of body lumens. Therefore, the urethra 1300 can be effectively supported. This effect is hereinafter described in detail.

Note that although the inclination angle θ1 is not limited particularly only if it is an acute angle, it is preferably, for example, approximately 20 to 60 degrees, more preferably 30 to 45 degrees, and most preferably 35 to 40 degrees. The effect described above is thereby further enhanced.

Although it is preferable for the inclination angle θ1 to satisfy the aforementioned numerical range over the overall region in the extending direction of the main body 31, only it is necessary for the aforementioned numerical range to be satisfied at least at the central portion S4 in the extending direction of the main body 31 so that the aforementioned effect can be demonstrated. Note that the “central portion S4” signifies a region including at least a portion positioned between the urethra 1300 and the vagina (vaginal cavity) 1400, which is one of body lumens similarly to the urethra 1300, in a state in which a living body is punctured by the puncture member 3 (a state in which the main body 31 is disposed inside the living body). Further, in the present embodiment, also it can be regarded that the central portion S4 is a middle portion between the anchors 81 and 82 (the center and portions in the proximity of the center) in a state in which the anchors 81 and 82 are engaged with the puncture member 3 as hereinafter described.

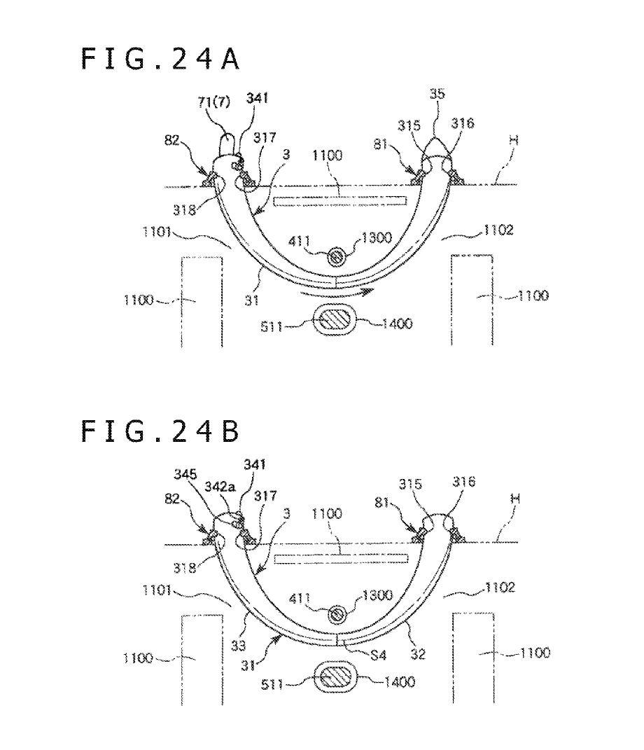

Note that a marker may be provided at portions, which protrude to the outside of a living body in a state in which the main body 31 is disposed in the living body (state of FIGS. 24A and 24B), of the opposite end portions of the main body 31 at positions spaced by an equal distance from the central portion S4, which makes it possible to confirm the position of the central portion S4 inside the living body by comparing the positions of both markers.

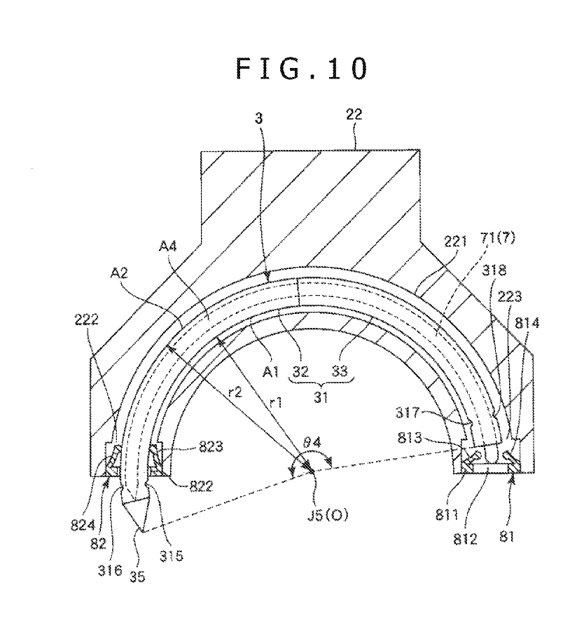

If the configuration of the main body 31 is represented in different words, it can be described in the following manner. In particular, the main body 31 is formed such that the major axis J32 is inclined with respect to the center axis J5 of the arc and the center axis J5 of the arc and an extension line J32′ of the major axis J32 have an intersection P as depicted in FIG. 4B. In this case, the angle θ5 defined between the center axis J5 and the extension line J32′ is equal to the inclination angle θ1. Alternatively, stated another way, also it can be regarded that, in a plan view as viewed from the direction of the center axis J5 of the main body 31 as depicted in FIG. 10, the main body 31 has the inner circumferential portion A1 positioned on an inner circumferential edge thereof and having a minimum radius of curvature r1 and the outer circumferential portion A2 positioned on an outer circumferential edge thereof and having a maximum radius of curvature r2. Further, as depicted in FIG. 4B, the inner circumferential portion A1 and the outer circumferential portion A2 can be positioned in a spaced apart (displaced) relationship from each other in the direction of the center axis J5.

The main body 31 having such a shape as described above is configured such that two divisional pieces are connected to each other such that the main body 31 can be divided into, for example, two or at the halfway point. In particular, the main body 31 has a distal end divisional piece 32 and a proximal end divisional piece 33. In accordance with an exemplary embodiment, the distal end divisional piece 32 and the proximal end divisional piece 33 have lengths substantially equal to each other, and the boundary between them is positioned at the central portion S4.

As depicted in FIG. 5, the distal end divisional piece 32 has a tubular shape and has a distal side opening 321 and a proximal side opening 322. In addition, the proximal end divisional piece 33 has a tubular shape and has a distal side opening 331 and a proximal side opening 332. In accordance with an exemplary embodiment, a distal end portion of the proximal end divisional piece 33 is inserted in a proximal end portion of the distal end divisional piece 32, whereby the distal end divisional piece 32 and the proximal end divisional piece 33 are connected to each other. By inserting the proximal end divisional piece 33 in the distal end divisional piece 32 in this manner, a step which may appear on the boundary between the divisional pieces 32 and 33 is less likely to be caught by the living body tissue. Consequently, puncture of a living body by the puncture member 3 can be performed relatively smoothly. However, conversely to the present embodiment, the distal end divisional piece 32 may be inserted in the proximal end divisional piece 33 to thereby connect the divisional pieces 32 and 33 together.

The connected state in which the divisional pieces 32 and 33 are connected together is maintained by the state maintaining mechanism 34. As depicted in FIG. 6A, the state maintaining mechanism 34 has holes 342a, 342b and 342c, an endless string (interlock member) 341 threaded in the holes 342a, 342b and 342c, exposure holes (through-holes) 345 and 346 for exposing the string 341, and a slit 347 interconnecting the exposure holes 345 and 346.

In accordance with an exemplary embodiment, the hole 342a is provided at a proximal end portion of the proximal end divisional piece 33 near the inner circumferential portion A1 of the front face A3. On the other hand, the holes 342b and 342c can be provided in an opposing relationship to each other at a position of the front face A3 and the back face A4 of a proximal end portion of the distal end divisional piece 32 rather near to the inner circumferential portion A1.

The string 341 is disposed in the main body 31 and is exposed to the outside of the main body 31 between the hole 342b and the hole 342c and between the hole 342a and the proximal side opening 332. By routing the string 341 in this manner, the connected state of the divisional pieces 32 and 33 can be maintained with certainty. Further, exposure of the string 341 to the outside of the main body 31 can be suppressed, and the string 341 becomes less likely to be caught by a living body tissue. Further, while such cutting of the string 341 as hereinafter described is made possible, the total length of the string 341 can be minimized. Therefore, when the implant main body 91 is threaded into the main body 31, the string 341 becomes less likely to be caught by the implant main body 91. Further, since the holes 342a, 342b and 342c are disposed near the inner circumferential portion A1 as aforementioned, also the string 341 is disposed rather near to the inner circumferential portion A1. Therefore, when the implant main body 91 is inserted into the main body 31, the string 341 becomes less likely to be caught by the implant main body 91.

The string 341 can be obtained, for example, by preparing a string having ends, inserting one end of the string into the main body 31 via the proximal side opening 332, drawing out the same to the outside of the main body 31 through the hole 342b, inserting the same into the main body 31 via the hole 342c, drawing out the same to the outside of the main body 31 through the hole 342a and tying the same with the other end of the string in the proximity of the proximal side opening 332. However, note that the position of the knot is not limited.

Here, as depicted in FIG. 6C, the axis of the hole 342a can be inclined such that the outer side opening is positioned on the proximal side with respect to the inner side opening. On the other hand, as depicted in FIG. 6B, the axis of each of the holes 342b and 342c is inclined such that the outer side opening is positioned on the distal side with respect to the inner side opening, which helps makes it possible to allow each of the holes 342a, 342b and 342c to extend along the route of the string 341. Consequently, the string 341 becomes less likely to be caught by the holes 342a, 342b and 342c.

In accordance with an exemplary embodiment, the exposure holes 345 and 346 are provided in an opposing relationship to the front face A3 and the back face A4 of the proximal end portion of the proximal end divisional piece 33, respectively. The locations at which the exposure holes 345 and 346 are provided project from the body surface H in a state in which the main body 31 is disposed in a living body. Further, the exposure holes 345 and 346 can be positioned on the route of the string 341. Therefore, the string 341 is exposed to the outside of the main body 31 from the exposure holes 345 and 346. Further, the exposure holes 345 and 346 can be tied with each other by the slit 347 provided on the inner circumferential portion A1 along a circumferential direction of the main body 31.

With such a state maintaining mechanism 34 as described above, if the string 341 is cut, then a state in which the distal end divisional piece 32 and the proximal end divisional piece 33 are separable from each other can be established. By the configuration, the distal end divisional piece 32 and the proximal end divisional piece 33 can be placed into a separable state through a relatively simple operation. Further, since the cutting of the string 341 is visually observable, it can be simply confirmed that the distal end divisional piece 32 and the proximal end divisional piece 33 are in a separable state.

By providing the exposure holes 345 and 346 and the slit 347 in such a manner as in the present embodiment, the string 341 can be cut relatively simply. Describing by way of example, scissors having a pair of blades (first blade and second blade) are prepared, and the first blade is inserted into the exposure holes 345 and 346 and the string 341 is positioned between the pair of blades. Then, if the scissors are operated to close, then at least one of the first and second blades passes through the slit 347 until the first and second blades are overlapped with each other, and the string 341 is cut during the process. By providing the exposure holes 345 and 346 and the slit 347 in this manner, the string 341 can be simply cut.

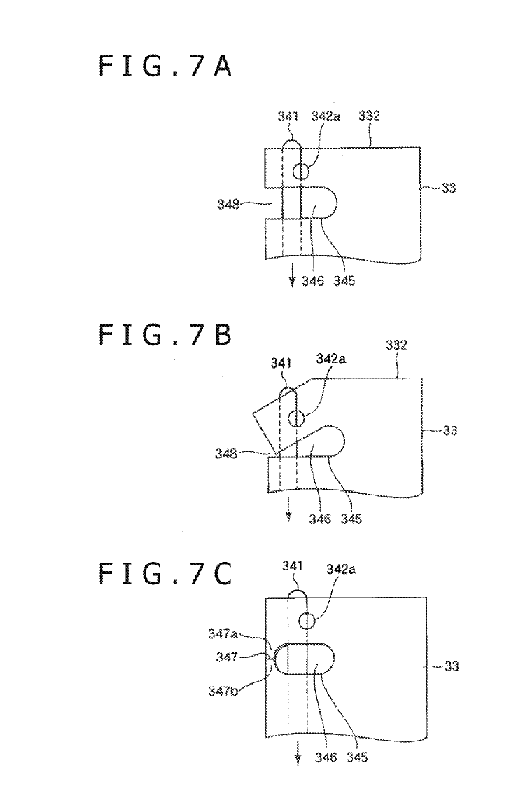

In this manner, in the present embodiment, the slit 347 is provided and used as a passing route of a blade. By this, deformation of the main body 31 by tension of the string 341 can be prevented. In particular, the passing route of a blade may be configured through a hole 348 in place of the slit 347 as depicted in FIG. 7A. In this case, however, depending upon the hardness of the main body 31, the hole 348 may be crushed through buckling by the tension on the string 341 as depicted in FIG. 7B, resulting in deformation of the main body 31. In contrast, in the case of the slit 347, since portions 347a and 347b on the opposite sides of the slit 347 attach to and are pressed against each other, such deformation as aforesaid will not occur as depicted in FIG. 7C. Consequently, the main body 31 can be prevented from deformation.

Further, as depicted in FIG. 5, the main body 31 has a pair of engaging holes 315 and 316 provided at a distal end portion thereof for engaging with the anchor 81. Meanwhile, the main body 31 has a pair of engaging holes 317 and 318 provided at a proximal end portion thereof for engaging with the anchor 82. From among the four engaging holes, the engaging holes 315 and 317 are provided on the inner circumferential portion A1 and the engaging holes 316 and 318 are provided on the outer circumferential portion A2.

As aforementioned, the main body 31 has a flattened shape and is less likely to be crushed in the major axis direction, and therefore, the separated distance between the inner circumferential portion A1 and the outer circumferential portion A2 is less likely to vary. Further, the inner circumferential portion A1 and the outer circumferential portion A2 are relatively large in curvature in comparison with the front face A3 and the back face A4 and are less likely to be deformed. Therefore, by providing the engaging holes 315 and 317 on the inner circumferential portion A1 and providing the engaging holes 316 and 318 on the outer circumferential portion A2, the engagement between the anchors 81 and 82 and the main body 31 is less likely to be cancelled.

Further, the separated distance between the engaging holes 315 and 316 and the central portion S4 and the separated distance between the engaging holes 317 and 318 and the central portion S4 are substantially equal to each other. Consequently, the anchors 81 and 82 play a role of markers, and the position of the central portion S4 of the main body 31 in a living body can be known.

Such a main body 31 as described above has the needle body 35 provided at the distal end of the main body 31. As depicted in FIG. 5, the needle body 35 can include a tapering needle tip 351, and a proximal end portion 352 provided on the proximal side of the needle tip 351. The proximal end portion 352 is inserted in the main body 31, whereby the needle body 35 is held on the main body 31 in a freely detachable manner. Note that the proximal end portion 352 is fitted in the main body 31 with force of such a degree that the needle body 35 can be prevented from being unintentionally removed from the main body 31. Note that the needle body 35 may be configured integrally with the main body 31.

Further, an engaging portion 353 for engaging with the distal end portion 711 of the insertion portion 71 is provided at the proximal end portion 352. The engaging portion 353 is configured from a recessed portion, and in an insertion state in which the insertion portion 71 is inserted in the puncture member 3, the distal end portion 711 is positioned in the engaging portion 353. By providing the engaging portion 353, displacement of the needle body 35 with respect to the insertion portion 71 can be reduced, and puncture of a living body by the puncture member 3 can be performed with increased smoothness.

The puncture member 3 has been described above. The center angle θ4 of the puncture member 3 is not limited particularly and is appropriately set in accordance with various conditions. However, the center angle θ4 is set such that the needle body 35 can enter the body of a patient via one of the inguinal regions, pass the region between the urethra 1300 and the vagina 1400 and project from the body via the other inguinal region. In particular, the center angle θ4 is preferably, for example, 150 to 270 degrees, more preferably 170 to 250 degrees, and most preferably 190 to 230 degrees.

The constituent materials of the main body 31 and the needle body 35 are preferably such rigid materials as may maintain the shape and the internal space of them in a state in which the puncture member 3 is inserted in a living body. As such rigid materials, for example, various resin materials such as polyethylene, polyimide, polyamide, polyester elastomer or polypropylene or various metal materials such as stainless steel, aluminum or aluminum alloy, or titanium or titanium alloy can be used. Note that, where the main body 31 and the needle body 35 are configured not by adopting rigid materials but by adopting other materials than rigid materials, the wall may be reinforced by a reinforcement member to achieve the same effect. For example, a braid of high strength may be embedded in the wall to maintain the shape and the internal space in the state in which the puncture member 3 is inserted in a living body. Another example of the reinforcement member is a spiral body, which is embedded in the wall of the main body 31, by which flexibility can be provided while the internal space is maintained to such a degree that an inserted article can be slidably moved therein.

In accordance with an exemplary embodiment, the main body 31 is preferably optically transparent so that the inside thereof can be visually observed from the outside. This makes it possible, for example, to confirm whether the distal end portion 711 of the insertion portion 71 inserted in the inside thereof is in engagement with the engaging portion 353, and whether or not the string 341 is broken.

A medical tube assembly 10 is configured from the puncture member 3 (main body 31) described above and the insertion portion 71 inserted in the main body 31, and use of the puncture apparatus 1 is started in a state in which they have the state of the medical tube assembly 10.

Note that the number and the disposition of holes (342a, 342b and 342c) through which the string 341 is to be threaded are not limited particularly only if the connected state of the distal end divisional piece 32 and the proximal end divisional piece 33 can be maintained by the string 341. Further, the string 341 need not have an endless state but may be in an ended state in which it has one end and the other end. For example, an ended string may be prepared and threaded at one end thereof through the hole 342a and the proximal side opening 332 to form a loop while it is threaded at the other end thereof through the holes 342b and 342c to form a loop. Further, the string 341 can include a cord and/or a belt, which can be used similarly to the string 341.

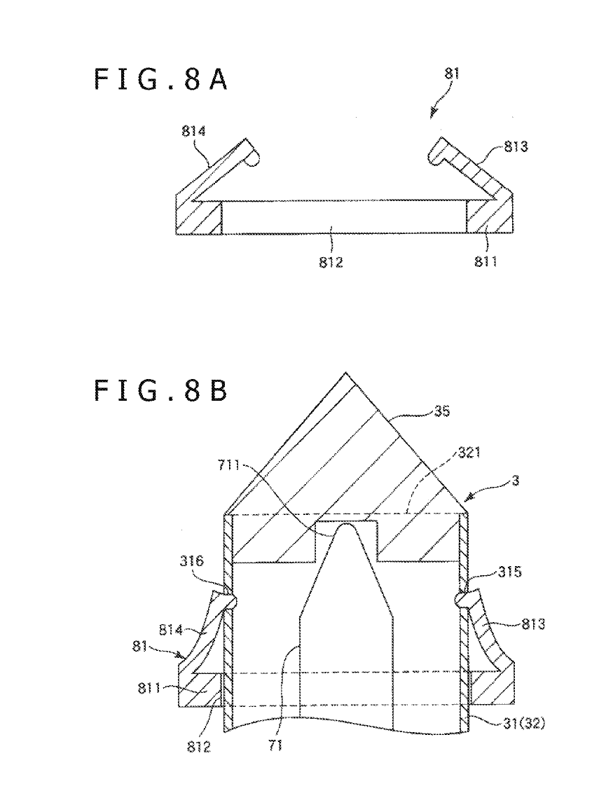

As depicted in FIG. 8A, the anchor (second anchor) 81 has a base portion 811 having therein a fitting hole 812 in which the main body 31 is to be fitted, and a pair of pawl portions 813 and 814 projecting from the base portion 811 and provided for engagement with the pair of engaging holes 315 and 316, respectively. The fitting hole 812 has a transverse sectional shape corresponding to the transverse sectional shape of the main body 31 of the puncture member 3. Therefore, in the state in which the puncture member 3 is fitted in the fitting hole 812, rotation of the anchor 81 with respect to the puncture member 3 can be restricted, and consequently, a positional relationship between them can be appropriately maintained. If the puncture member 3 is fitted into the fitting hole 812 and then advanced with respect to the anchor 81, then the pawl portions 813 and 814 are engaged with the engaging holes 315 and 316, respectively, as depicted in FIG. 8B. Consequently, the anchor 81 is engaged with the distal end divisional piece 32. In the state in which the anchor 81 engages with the distal end divisional piece 32, the base portion 811 is positioned on the proximal side with respect to the pawl portions 813 and 814. In the state in which the puncture member 3 is fitted in the fitting hole 812, rotation of the anchor 81 with respect to the puncture member 3 is restricted as aforementioned. Therefore, engagement between the pawl portions 813 and 814 and the engaging holes 315 and 316 can be established with relative certainty.

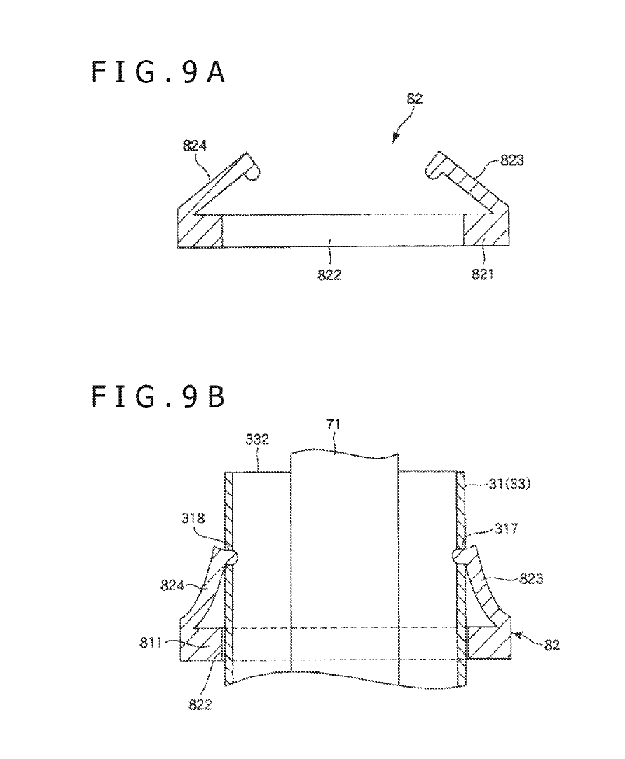

Similarly, as depicted in FIG. 9A, the anchor (first anchor) 82 has a base portion 821 having therein a fitting hole 822 in which the main body 31 is to be fitted, and a pair of pawl portions 823 and 824 projecting from the base portion 821 and provided for engagement with the pair of engaging holes 317 and 318, respectively. The fitting hole 822 has a transverse sectional shape corresponding to the transverse sectional shape of the main body 31. Therefore, in the state in which the puncture member 3 is fitted in the fitting hole 822, rotation of the anchor 82 with respect to the puncture member 3 can be restricted, and consequently, a positional relationship between them can be appropriately maintained. If the puncture member 3 is fitted into the fitting hole 822 and then advanced with respect to the anchor 82, then the pawl portions 823 and 824 can be engaged with the engaging holes 317 and 318, respectively, as depicted in FIG. 9B. Consequently, the anchor 82 is engaged with the proximal end divisional piece 33. In the state in which the anchor 82 engages with the proximal end divisional piece 33, the base portion 821 is positioned on the distal side with respect to the pawl portions 823 and 824. In the state in which the puncture member 3 is fitted in the fitting hole 822, rotation of the anchor 82 with respect to the puncture member 3 is restricted as aforementioned. Therefore, engagement between the pawl portions 823 and 824 and the engaging holes 317 and 318 can be established with relative certainty.

The constituent materials for the anchors 81 and 82 are not limited particularly, and, for example, various resin materials can be used.

The frame 2 holds the operation member 7, on which the puncture member 3 is mounted, for rotary movement and has an insertion tool 6 and the anchors 81 and 82 fixed thereto in a freely detachable manner. The frame 2 has a function of determining, when the puncture member 3 is to puncture a living body tissue, a puncture route of the needle body 35. In particular, the frame 2 defines a positional relationship among the puncture member 3, urethral insertion member 4 and vaginal insertion member 5 such that, when the puncture member 3 punctures a living body tissue, the needle body 35 passes a region between the urethral insertion member 4 and the vaginal insertion member 5 without interfering with them.

As depicted in FIGS. 1 and 2, the frame 2 has a bearing portion 21, a guide portion (holding portion) 22, an interlock portion 23 and a fixation portion 24. The bearing portion 21 supports the shaft portion 73 of the operation member 7 for rotation thereon. The guide portion 22 guides the puncture member 3 and holds the first and second anchors 81 and 82 thereon in a freely detachable manner. The interlock portion 23 interlocks the bearing portion 21 and the guide portion 22 to each other. The fixation portion 24 is used such that the insertion tool 6 is fixed thereto.

The bearing portion 21 is positioned on the proximal side of the puncture apparatus 1 and extends substantially orthogonally to the axis J1. A through-hole 211 is formed in the bearing portion 21 on the axis J1, and the shaft portion 73 is inserted for rotation in the through-hole 211. Consequently, the operation member 7 is supported on the frame 2 for rotary movement around the axis J1.

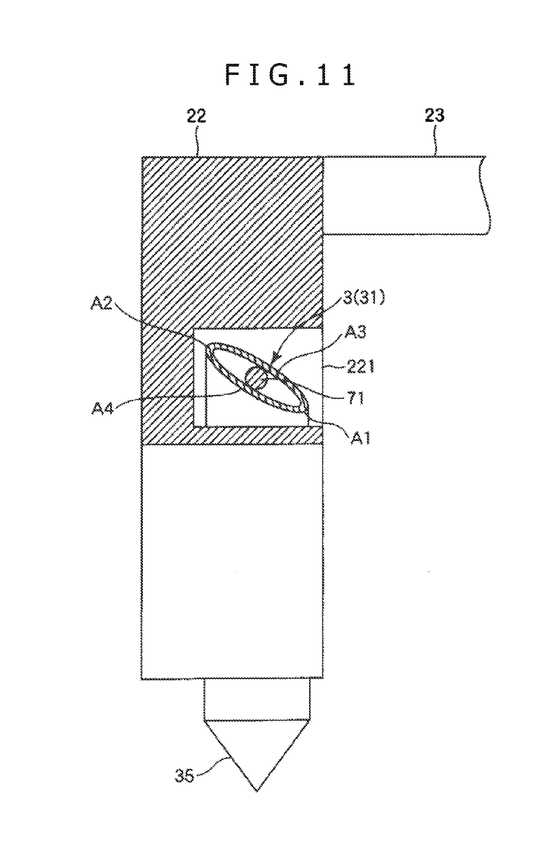

The guide portion 22 is positioned on the distal side of the puncture apparatus 1 and disposed in an opposing relationship to the bearing portion 21. As depicted in FIG. 10, the guide portion 22 has a substantially C-shaped guide groove 221 formed thereon so as to accommodate and guide the puncture member 3. Further, as depicted in FIG. 11, in a state in which the puncture member 3 is disposed in the guide groove 221, the puncture member 3 has the back face A4 positioned on the distal side and has the front face A3 positioned on the proximal side.

Further, the guide portion 22 holds the anchors 81 and 82 thereon in a freely detachable manner. The anchor 82 is held in an opposing relationship to a distal side opening 222 of the guide groove 221 such that the fitting hole 822 continues to the guide groove 221. Meanwhile, the anchor 81 is held in an opposing relationship to a proximal side opening 223 of the guide groove 221 such that the fitting hole 812 continues to the guide groove 221.

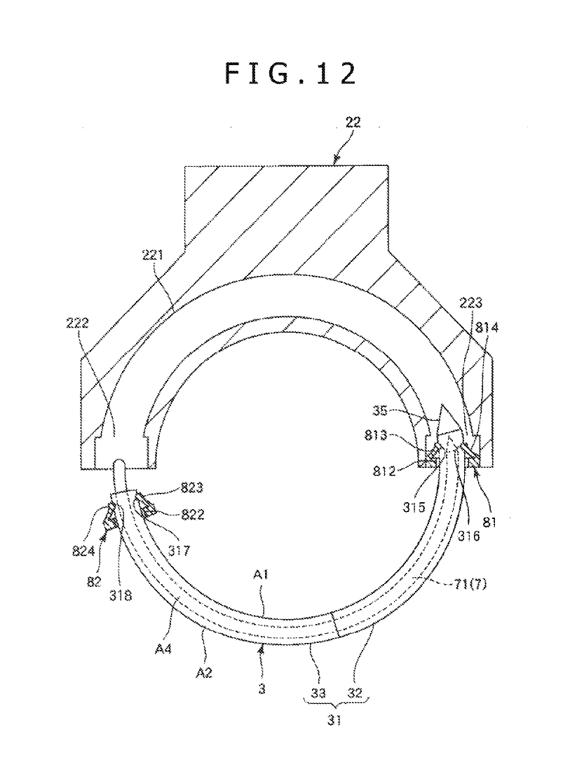

In the initial state, the main body 31 is fitted in the fitting hole 822 of the anchor 82 and besides the needle body 35 projects from the guide portion 22. Thus, if the operation member 7 is operated to rotate, then the puncture member 3 gradually projects from the guide portion 22 until the needle body 35 finally enters the guide portion 22 through the proximal side opening 223 as depicted in FIG. 12. In this procedure, on the distal side of the puncture member 3, the puncture member 3 passes through the fitting hole 812 of the anchor 81 and the pawl portions 813 and 814 are engaged with the engaging holes 315 and 316, respectively. On the other hand, on the proximal side of the puncture member 3, the pawl portions 823 and 824 are engaged with the engaging holes 317 and 318, respectively. Consequently, the anchors 81 and 82 are engaged with the puncture member 3.

The interlock portion 23 interlocks the bearing portion 21 and the guide portion 22 to each other. Further, the interlock portion 23 has a shape of a bar extending substantially in parallel to the axis J1. The interlock portion 23 can also function as a grasping portion, and the operator can grasp the interlock portion 23 to use the puncture apparatus 1.

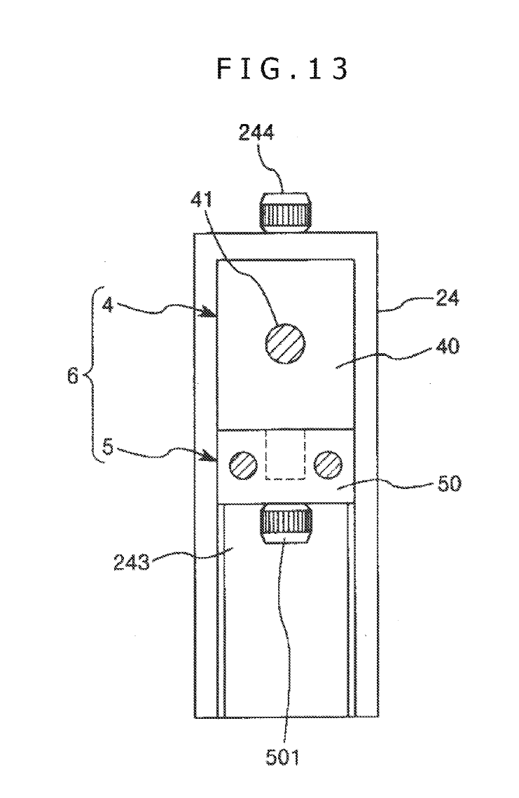

The fixation portion 24 is disposed in an opposing relationship with the interlock portion 23 with the axis J1 interposed therebetween. As depicted in FIG. 13, the fixation portion 24 has a recessed portion 243 into which a support unit 60 hereinafter described of the insertion tool 6 is to be fitted, and a male screw 244. By fitting the support unit 60 into the recessed portion 243 and tightening the male screw 244 into a female screw (not depicted) of the support unit 60, the insertion tool 6 can be fixed to the fixation portion 24.

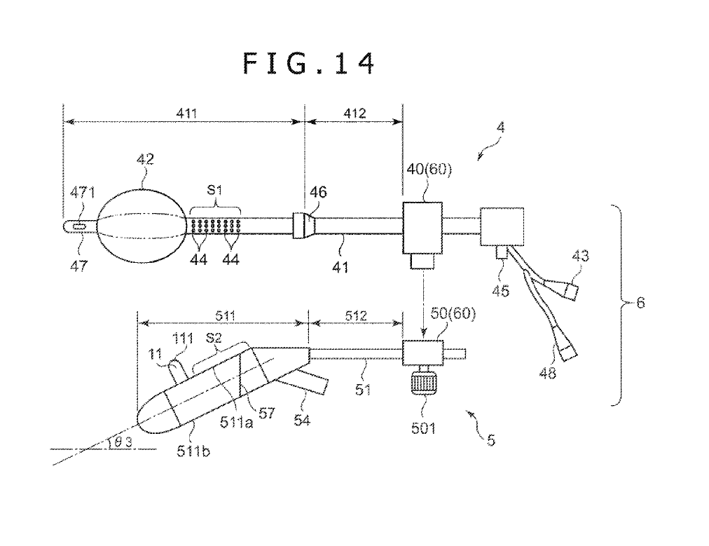

As depicted in FIGS. 1 and 14, the insertion tool 6 has a urethral insertion portion (second insertion portion) 41 for being inserted into the urethra 1300, a vaginal insertion portion (first insertion portion) 51 for being inserted into the vagina 1400, and the support unit 60 for supporting the urethral insertion portion 41 and the vaginal insertion portion 51 in a juxtaposed relationship thereon. As aforementioned, the insertion tool 6 is configured from two living body insertion tools, namely, the urethral insertion member 4 and the vaginal insertion member 5 as described hereinabove. Further, the urethral insertion member 4 can include the urethral insertion portion 41 while the vaginal insertion member 5 can include the vaginal insertion portion 51. Further, the support unit 60 has a support portion 40 provided on the urethral insertion member 4 for supporting the urethral insertion portion 41 and a support portion 50 provided on the vaginal insertion member 5 for supporting the vaginal insertion portion 51. In the insertion tool 6, the urethral insertion member 4 and the vaginal insertion member 5 are freely detachable from each other through the support portions 40 and 50. In the following, the urethral insertion member 4 and the vaginal insertion member 5 are described in order.

The urethral insertion member 4 has the elongate urethral insertion portion 41 for being inserted to an intermediate portion thereof into the urethra 1300, and the support portion 40 for supporting the urethral insertion portion 41. Note that, in the following description, for the convenience of description, a portion of the urethral insertion portion 41 which is positioned in the urethra 1300 (including the bladder 1310) in the mounted state is referred to also as “insertion portion 411.” Meanwhile, another portion of the urethral insertion portion 41, which is exposed to the outside of the body from the urethral orifice in the mounted state and extends to the support portion 40, is referred to also as “non-insertion portion 412.”

The urethral insertion portion 41 has a straight tubular shape rounded at the distal end thereof. Further, an expandable-contractible balloon 42 and a urine drainage portion 47 can be provided at a distal end portion of the insertion portion 411. As depicted in FIG. 19, the balloon 42 can function as a restriction portion (or “attaching portion”) which is expanded, in a state in which the urethral insertion portion 41 is inserted in the urethra 1300 until the urine drainage portion 47 reaches the bladder 1310, and attaches to the bladder neck 1320 to restrict the position of the urethral insertion member 4 in the axial direction in the urethra 1300. In particular, when the puncture apparatus 1 is to be used, the balloon 42 is expanded after it is inserted into the bladder 1310 of the patient. Then, since the expanded balloon 42 is caught by the bladder neck 1320, the position of the urethral insertion member 4 with respect to the bladder 1310 and the urethra 1300 is fixed. Meanwhile, the urine drainage portion 47 has a function for draining the urine in the bladder 1310 and further has a function for supplying liquid such as water into the bladder 1310.

The balloon 42 is connected to a balloon port 43 provided at a proximal end portion of the urethral insertion portion 41 through the inside of the urethral insertion portion 41. A balloon expansion tool such as, for example, a syringe can be connected to the balloon port 43. If working fluid (fluid like physiological saline solution, or gas) is supplied from the balloon expansion tool into the balloon 42, then the balloon 42 is expanded. In contrast, if the working fluid is extracted from the balloon 42 by the balloon expansion tool, then the balloon 42 is contracted. Note that, in FIG. 14, the balloon 42 in a contracted state is indicated by a two-dot chain line, and the balloon 42 in an expanded state is indicated by a solid line.

Meanwhile, a drain hole 471 is provided in the urine drainage portion 47 such that it communicates the inside and the outside of the urine drainage portion 47 with each other. Further, the urine drainage portion 47 is connected to a urine drainage port 48 provided at the proximal end portion of the urethral insertion portion 41 through the inside of the urethral insertion portion 41. If a suction tool such as, for example, a syringe is connected to the urine drainage port 48 and is operated, and then the urine introduced from the drain hole 471 can be drained from the urine drainage port 48. In accordance with an exemplary embodiment, a syringe, in which, for example, water is filled, can be connected to the urine drainage port 48. In this case, by operating the syringe to press the plunger, water can be supplied into the bladder 1310 through the drain hole 471.

The balloon 42 and the urine drainage portion 47 can be configured, for example, from a double-lumen.

At an intermediate portion of the insertion portion 411, a plurality of suction holes (suction ports) 44 are formed such that they are open to an outer circumferential face of the insertion portion 411. The suction holes 44 are disposed over an overall area of the urethral insertion portion 41 in the circumferential direction. The suction holes 44 are connected to a suction port 45 provided at the proximal end portion of the urethral insertion portion 41 through the urethral insertion portion 41. A suction device such as a pump can be connected to the suction port 45. Then, if the urethral insertion portion 41 is inserted into the urethra 1300, then the outer circumferential face of the insertion portion 411 of the urethral insertion portion 41 is placed into a state in which it attaches (or is positioned closely) to the living body tissue between the urethra 1300 and the vagina 1400, namely, to the urethral wall. If the suction device is rendered operative in this state, then the urethral wall is sucked to the outer circumferential face side of the insertion portion 411. Consequently, the closeness of contact between the urethral wall and the outer circumferential face of the insertion portion 411, which is an attachment face to the urethral wall, can be raised. If the urethral insertion portion 41 is pushed in to the distal side (into the body) in this state, then also the urethra 1300 is pushed in together. Consequently, for example, the bladder 1310 can be displaced to a position at which it does not overlap with the puncture route of the puncture member 3, and the puncture route of the puncture member 3 can be assured. Therefore, puncture of the puncture member 3 can be performed accurately and safely.

Note that the number of suction holes 44 is not limited particularly and may be, for example, one. The disposition of the suction holes 44 is not limited particularly, and the suction holes 44 may be formed, for example, only at part of the urethral insertion portion 41 in the circumferential direction.

Further, in the puncture apparatus 1, a line from each suction hole 44 to the suction device such as a pump past the suction port 45 is sometimes referred to as “suction line 49.”

As depicted in FIG. 14, a ring member 46 is mounted on the urethral insertion portion 41 such that it divides the urethral insertion portion 41 into the insertion portion 411 and the non-insertion portion 412. The ring member 46 is movable along a longitudinal direction of the urethral insertion portion 41 and is supported for fixation at the destination of the movement. The ring member 46 functions, for example, as a marker for confirming the insertion depth of the urethral insertion portion 41 into the urethra 1300 and functions also as a cap for sealing the urethral orifice in a state in which the urethral insertion portion 41 is inserted in the urethra 1300.

The length of the insertion portion 411 is not limited particularly and is suitably set depending upon the length of the urethra 1300 of the patient, the shape of the bladder 1310 and so forth. Since the length of the urethra of general women is approximately 30 to 50 mm, preferably the length of the insertion portion 411 is approximately, for example, 50 to 100 mm.

Although the length of the non-insertion portion 412 (separated distance between the urethral orifice and the support portion 40) is not limited particularly, it is preferably equal to or smaller than, for example, approximately 100 mm, and more preferably approximately 20 to 50 mm. Thus, the non-insertion portion 412 can be made having an appropriate length, which improves the operability. If the length of the non-insertion portion 412 exceeds the aforesaid upper limit value, then depending upon the configuration or the like of the frame 2, the center of gravity of the puncture apparatus 1 may be displaced by a great distance from the patient and the stability of the puncture apparatus 1 in the mounted state may be degraded.

The constituent material of the urethral insertion member 4 is not limited particularly, and various metal materials such as, for example, stainless steel, aluminum or aluminum alloy, or titanium or titanium alloy or various resin materials can be used.

Here, the inclination angle θ2 of the plane f9 (plane f1) with respect to the plane f2 orthogonal to the axis J2 of the urethral insertion portion 41 is preferably, for example, approximately 20 to 60 degrees, more preferably approximately 30 to 45 degrees, and most preferably 35 to 40 degrees. In accordance with an exemplary embodiment, the main body 31 is preferably indwelled in the body such that the angle defined between the plane f9 and the plane orthogonal to the axis of the urethra 1300 becomes, for example, approximately 20 to 60 degrees, more preferably indwelled in the body such that the angle becomes approximately 30 to 45 degrees, and most preferably indwelled in the body such that the angle becomes 35 to 40 degrees. Where the main body 31 is indwelled in this manner, puncture by the puncture member 3 can be performed readily and the puncture distance by the puncture member 3 can be reduced.

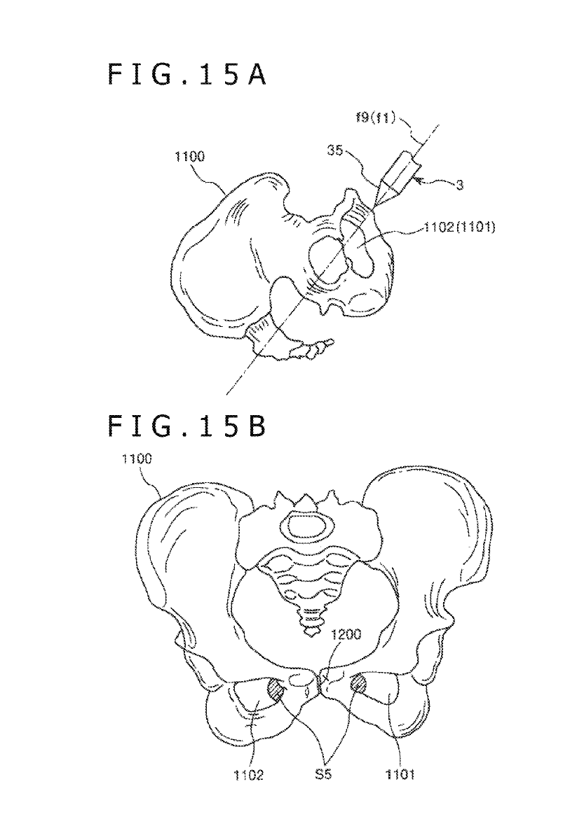

Describing more particularly, if the inclination angle θ2 is set within the aforesaid range, then the puncture member 3 can capture the left and right obturator foramens 1101 and 1102 of the pelvis 1100 widely in a plane as depicted in FIG. 15A, and a wide puncture space can be assured for the puncture member 3. In other words, in a state in which the patient assumes a predetermined posture (lithotomy position), the puncture member 3 can be caused to puncture in a comparatively vertical direction with respect to the obturator foramens 1101 and 1102. Therefore, puncture by the puncture member 3 can be performed. In addition, where the puncture member 3 can puncture the obturator foramens 1101 and 1102 in a comparatively vertical direction, since the puncture member 3 passes a comparatively shallow portion of the tissue, the needle body 35 of the puncture member 3 can pass the location between the left and right obturator foramens 1101 and 1102 in a shorter distance. Therefore, the puncture member 3 can pass rather close to the pubic symphysis 1200 with respect to the obturator foramens 1101 and 1102, preferably pass a safety zone S5 as depicted in FIG. 15B. Since the safety zone S5 is a region, which can include a comparatively small number of nerves or blood vessels to which damage is to be avoided, the puncture member 3 can be caused to puncture in safe. Therefore, the puncture becomes less invasive, and the burden on the patient can be reduced. In this manner, by setting the inclination angle θ2 to the aforesaid range, puncture of the patient by the puncture member 3 can be appropriately performed. Further, by puncturing the patient by the angle given above, it becomes easy to target the tissue between the central urethra, which represents a central portion of the urethra 1300 in the lengthwise direction, and the vagina 1400. The location between the central urethra and the vagina 1400 is a position suitable as a portion into which the implant 9 can be embedded to perform treatment of urinary incontinence.

In contrast, where the inclination angle θ2 is less than the aforesaid lower limit value or is greater than the aforesaid upper limit value, depending upon the individual difference of the patient, the posture during the manipulation, the puncture member 3 may not be able to capture the obturator foramens 1101 and 1102 widely in a plane or may not be able to sufficiently shorten the puncture route.

More preferably, puncture is performed in a state in which the position of the urethra 1300 or the vagina 1400 or the positions of both of the urethra 1300 and the vagina 1400 are displaced so as to be pushed in to the inner side of the body. This makes it easy to puncture the region between the central urethra and the vagina 1400. The method for pushing one of the urethra 1300 and the vagina 1400 into the inner side of the body may be, for example, a method of placing the urethral insertion member 4 and/or the vaginal insertion member 5 into a state in which they are inserted to appropriate positions, sucking the urethra 1300 and/or the vagina 1400 to and by the suction holes 44 and 59 hereinafter described which are provided on the urethral insertion member 4 and/or the vaginal insertion member 5 and then further moving the urethral insertion member 4 and/or the vaginal insertion member 5 to predetermined positions in the inside of the body along respective axial lines. By causing, in the state in which at least one of the urethra 1300 and the vagina 1400 is positionally displaced so as to be pushed in to the inner side of the body in this manner, the main body 31 to puncture the region between and perpendicularly to the left and right obturator foramens 1101 and 1102 of the pelvis 1100, a path can be formed at a position suitable for indwelling of the implant 9.

In accordance with an exemplary embodiment, it can be preferable to set the locus of the main body 31 so as to pass the safety zone S5 of the left and right obturator foramens 1101 and 1102 of the pelvis 1100, displace at least one of the urethra 1300 and the vagina 1400 to the inner side of the body so that the locus may be positioned between the central urethra and the vagina 1400, and cause the main body 31 to puncture along the locus to form the path.

As depicted in FIGS. 1 and 14, the vaginal insertion member 5 has the elongate vaginal insertion portion (first insertion portion) 51 for being inserted to an intermediate portion thereof into the vagina 1400, and the support portion 50 for supporting the vaginal insertion portion 51. Note that, in the following description, for the convenience of description, a portion of the vaginal insertion portion 51 which is positioned in the vagina 1400 in the mounted state is referred to also as “insertion portion 511,” and another portion of the vaginal insertion portion 51 which is exposed to the outside of the body from the vaginal orifice in the mounted state and extends to the support portion 50 is referred to also as “non-insertion portion 512.”

In accordance with an exemplary embodiment, the insertion portion 511 has an elongate form. Further, the insertion portion 511 extends in an inclined relationship with respect to the insertion portion 411 such that the distal side thereof is spaced apart from the insertion portion 411. Where the insertion portion 511 is inclined with respect to the insertion portion 411 in this manner, the positional relationship between the insertion portions 411 and 511 can be made close to the positional relationship between the urethra 1300 and the vagina 1400 in comparison with that in an alternative case in which the insertion portion 511 is not inclined. Therefore, in the mounted state, the puncture apparatus 1 can be held stably by the patient, and the burden on the patient is moderated. Although the inclination angle θ3 of the insertion portion 511 with respect to the insertion portion 411 is not limited particularly, it is preferably, for example, approximately 0 to 45 degrees, and more preferably approximately 0 to 30 degrees. This makes it possible to demonstrate the aforesaid effect more prominently. In contrast, where the inclination angle θ3 is less than the aforesaid lower limit value or greater than the aforesaid upper limit value, depending upon the individual difference of the patient, the posture during the manipulation, in the mounted state, the vagina 1400 or the urethra 1300 may be deformed unnaturally and the puncture apparatus 1 may not be held stably.

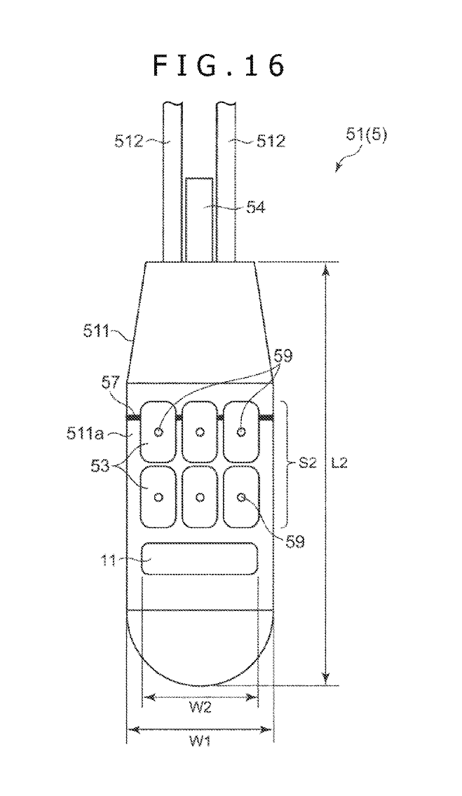

As depicted in FIG. 16, the insertion portion 511 has a flattened shape collapsed in an upward and downward direction of the puncture apparatus 1 (in a direction in which the urethra 1300 and the vagina 1400 are arrayed). Further, the insertion portion 511 has a central portion having a substantially fixed width and a rounded distal end portion. Although the length L2 of the insertion portion 511 is not limited particularly, it is preferably, for example, approximately 20 to 100 mm, and more preferably approximately 30 to 60 mm. Further, although the width W1 of the insertion portion 511 is not limited particularly, it is preferably, for example, approximately 10 to 50 mm, and more preferably 20 to 40 mm. Further, although the thickness of the insertion portion 511 is not limited particularly, it is preferably approximately 5 to 25 mm, and more preferably approximately 10 to 20 mm. Such length×width×thickness as disclosed above can provide a shape and a size suitable for the general vagina 1400 to the insertion portion 511. Therefore, the stability of the puncture apparatus 1 in the mounted state thereof is increased and the burden on the patient is reduced.

An upper face (face on the urethral insertion portion 41 side) 511a of the insertion portion 511 has a plurality of bottomed recessed portions 53 formed thereon and disposed in rows and columns. Note that the number of recessed portions 53 is not limited particularly and may be, for example, one. Further, a single suction hole 59 is provided on the bottom face of each of the recessed portions 53, and the suction holes 59 are connected to a suction port 54 provided at a proximal end portion of the insertion portion 511 through the inside of the insertion portion 511. The suction port 54 is provided so as to be positioned outside the living body in the mounted state. A suction device such as a pump can be connected to the suction port 54. Thus, if the vaginal insertion portion 51 is inserted into the vagina 1400, then the upper face 511a of the insertion portion 511 of the vaginal insertion portion 51 is placed into a state in which it attaches (or is positioned closely) to the living body tissue between the urethra 1300 and the vagina 1400, namely, to the vaginal wall. If, in this state, the suction device is rendered operative, then the vaginal wall is sucked to the upper face 511a side of the insertion portion 511. Consequently, the closeness of contact between the vaginal wall and the upper face 511a of the insertion portion 511, which is the attachment face to the vaginal wall, can be increased. If, in this state, the vaginal insertion portion 51 is pushed to the distal side (into the inner side of the body), then the vaginal wall can be pushed in together. Therefore, the disposition or the shape of the vaginal wall can be arranged and a puncture route for the puncture member 3 can be secured. Consequently, puncture by the puncture member 3 can be performed relatively accurately and safely.

Note that, although, in the present embodiment, one suction hole 59 is formed in each one recessed portion 53, the number of such suction holes 59 is not limited to this and a plurality of suction holes 59 may be formed in each one recessed portion 53. Further, the total number of suction holes 59 is not limited to a plural number but may be one.

Further, in the puncture apparatus 1, a line from each suction hole 59 to the suction device such as a pump through the suction port 54 is sometimes referred to as “suction line 58.”

As depicted in FIG. 14, a region S2 in which the plural recessed portions 53 are formed is disposed in an opposing relationship to a region S1 in which the suction holes 44 are formed. Further, the needle tip 351 of the puncture member 3 passes in the region between the regions S1 and S2. As aforementioned, in the region S1, the urethra 1300, which is a lower face of the urethral wall is sucked to the insertion portion 411, and in the region S2, the vaginal front wall is sucked to the insertion portion 511. Therefore, between the regions S1 and S2, the urethral wall and the vaginal wall are spaced apart by an increased distance from each other. Therefore, since the puncture member 3 passes such a region as just described, the puncture member 3 can puncture with a higher degree of safety.

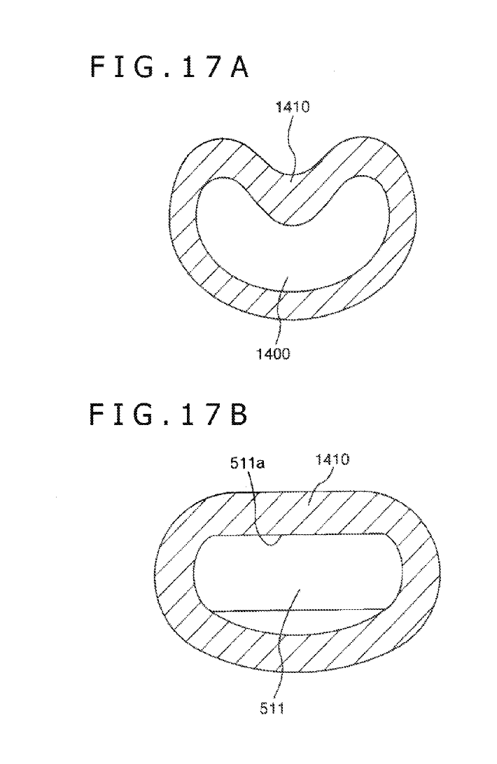

The region S2 extends over a substantially overall area of the upper face 511a in the widthwise direction. Although the width W2 of the region S2 is not limited particularly, it is preferably approximately, for example, 9 to 49 mm, and more preferably 19 to 39 mm. This makes it possible to allow the vaginal front wall to be sucked to the insertion portion 511 with a higher degree of certainty without being influenced much by the shape of the vaginal wall. Especially, for example, depending upon the patient, the vagina 1400 sometimes has such a shape that the vaginal front wall 1410 partly hangs down into the inside of the vagina 1400 as depicted in FIG. 17A. Also in such a case, if the region S2 has such a width W2 as aforesaid, then not only the hanging portion but also portions of the vagina 1400 on the opposite sides of the handing portion can be sucked with certainty as depicted in FIG. 17B. Therefore, the vaginal front wall can be spaced apart from the urethra 1300 with a higher degree of certainty without being influenced by the shape of the vagina 1400. Especially, for example, in the present embodiment, since the insertion portion 511 has a flattened shape, the vaginal front wall can be sucked so as to be spaced apart by a greater distance from the urethra 1300. Consequently, the living body tissue between the urethral wall and the vaginal wall can be expanded to a greater width.

As depicted in FIGS. 14 and 16, a protrusion 11 is disposed very closely to the distal side of the region S2, namely, on the distal side (forwardly in the insertion direction) with respect to the suction holes 59. The protrusion 11 protrudes toward the urethral insertion member 4 side and functions as an attachment detection portion for detecting whether or not the balloon 42 of the urethral insertion member 4 in an expanded state attaches to the bladder neck 1320. Note that the protrusion 11 is preferably, for example, rounded at the apex 111 thereof.

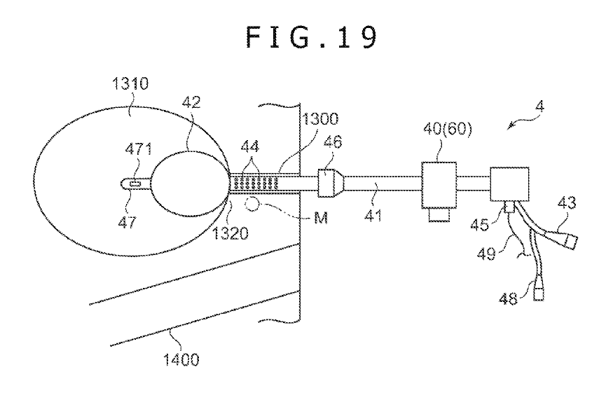

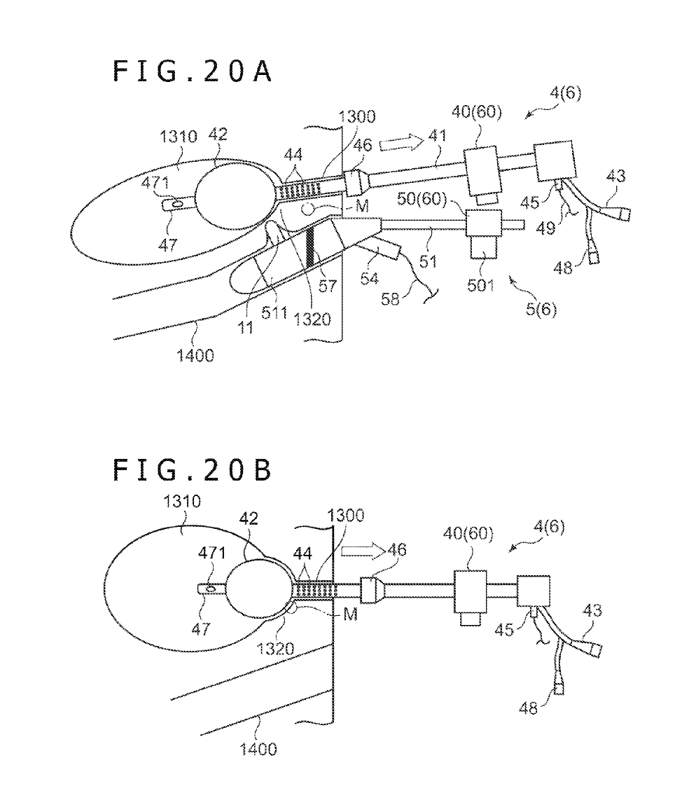

Incidentally, the urethral insertion member 4 is sometimes drawn in a direction toward the proximal end intending to expand the balloon 42 in a state in which the urethral insertion member 4 is inserted in the urethra 1300 and attach the balloon 42 to the bladder neck 1320 in order to position the urethral insertion member 4. In this case, for example, where the patient is an elderly woman and has the bladder neck 1320 comparatively emaciated, the balloon 42 pushes open the bladder neck 1320 and excessively enters the urethra 1300 as depicted in FIG. 20B. In the state depicted in FIG. 20B, a supposed puncture route M of the puncture member 3 in the living body tissue and the urethra 1300 (bladder neck 1320) interfere with each other, which is not preferable to the treatment for urinary incontinence of a woman. Further, such a state as described above is less likely to be recognized by the operator.

Therefore, it can be detected from the protrusion 11 whether or not the expanded balloon 42 of the urethral insertion member 4 attaches to the bladder neck 1320, namely, that the balloon 42 does not excessively enter the urethra 1300. This is described particularly below.

First, the urethral insertion member 4 is inserted into the urethra 1300 and the balloon 42 is expanded in the bladder 1310 as depicted in FIG. 19. Then, the urethral insertion member 4 is inserted into the urethra 1300, the vaginal insertion member 5 is inserted into the vagina 1400, and then the urethral insertion member 4 is pulled to the proximal side as depicted in FIG. 20A.

Note that the insertion depth of the vaginal insertion member 5 into the vagina 1400 is preferably set to a depth in which a marker 57 provided on the vaginal insertion member 5 is positioned on the interior side, for example, by approximately 15 mm from the vaginal orifice. At this time, the protrusion 11 is in a state in which it presses the proximity of the bladder neck 1320 toward the urethral insertion member 4 side.

Then, if the urethral insertion member 4 is pulled to the proximal side as aforementioned, then if the balloon 42 expanded to the protrusion 11 side does not excessively enter the urethra 1300 but attaches lightly to the bladder neck 1320, then oscillation upon the attachment is transmitted to the protrusion 11 through the bladder neck 1320 (living body tissue). From a result of the detection by the protrusion 11, the operator can decide and recognize that the balloon 42 is in attachment to the bladder neck 1320 and therefore the puncture route M is positioned on the urethral orifice side with respect to the bladder neck 1320, or in other words, the protrusion 11 as the detection portion is positioned on the distal side with respect to the puncture route M of the puncture member 3. Then, if puncture is performed in this state, then the needle body 35 of the puncture member 3 can be prevented from puncturing the urethra 1300 or the bladder 1310, namely, for example, occurrence of puncture in error can be prevented with relative certainty. In other words, when a surgical procedure such as puncture is performed for a living body tissue positioned between the urethra 1300 and the vagina 1400, the procedure can be performed with relative certainty.

Further, as depicted in FIG. 16, the protrusion 11 extends in a direction orthogonal to the longitudinal direction of the vaginal insertion member 5, namely, along a widthwise direction. Consequently, for example, in comparison with an alternative case in which the protrusion 11 has a form of a pin, a relatively large area can be secured as the area over which it is detected whether or not the balloon 42 attaches to the bladder neck 1320, and consequently, the detection can be performed with relative certainty.

Further, the insertion portion 511 has the marker (puncture position confirmation portion) 57 provided thereon such that the puncture route of the puncture apparatus 1 can be confirmed from the marker 57. Thus, the puncture apparatus 1 can be fixed such that it punctures the region between the vaginal wall and the urethral wall existing on an upper face of a position at which the marker 57 exists. Therefore, the operability and the safety of the insertion tool 6 are improved. The marker 57 is provided at least on a lower face 511b of the insertion portion 511. The lower face 511b is a face, which is directed to the vaginal orifice in the insertion state and can be visually recognized through the vaginal orifice by the operator. Therefore, by providing the marker 57 on the lower face 511b, the puncture route of the puncture apparatus 1 can be confirmed with a higher degree of certainty. In addition, the insertion depth of the insertion portion 511 in the vagina 1400 can also be confirmed. Note that only it is necessary for the marker 57 to be visually recognized from the outside, and the marker 57 can be configured, for example, from a colored portion, or a roughened portion.

The non-insertion portion 512 has a form of a thin bar extending substantially in parallel to the urethral insertion portion 41. Although the separated distance D between the non-insertion portion 512 and the urethral insertion portion 41 is not limited particularly, it is preferably, for example, approximately 5 to 40 mm so as to be suitable for the separated distance between the urethral orifice and the vaginal orifice of most women.

Although the length of the non-insertion portion 512 (separated distance between the vaginal orifice and the support portion 50) is not limited particularly, it is preferably, for example, equal to or smaller than approximately 100 mm, and more preferably approximately 20 to 50 mm. This makes it possible to set the non-insertion portion 512 to an appropriate length and improve the operability. If the length of the non-insertion portion 512 exceeds the aforesaid upper limit value, then depending upon the configuration of the frame 2 and so forth, the center of gravity of the puncture apparatus 1 may be spaced apart by a large distance from the patient, resulting in degradation of the stability of the puncture apparatus 1 in the mounted state.

A male screw 501 is provided on the support portion 50. If the male screw 501 is tightened to a female screw (not depicted) of the support portion 40, then the support portions 40 and 50 are fixed to each other.

The constituent material of the vaginal insertion member 5 is not limited particularly, and for example, various metal materials such as stainless steel, aluminum or aluminum alloys, and titanium or titanium alloys or various resign materials can be used similarly to the urethral insertion member 4.

Further, the support portion 40 supports the urethral insertion portion 41 for movement in the longitudinal direction of the urethral insertion portion 41. Consequently, in the state in which the support portion 40 and the support portion 50 are interlocked to each other, the protrusion 11 and the balloon 42 can move toward and away from each other (toward and/or away from each other).

The puncture apparatus 1 has the configuration described above.

Note that, while the urethral insertion member 4 and the vaginal insertion member 5 which configure the insertion tool 6 in the puncture apparatus 1 are configured so as to be freely detachable, the configuration of the urethral insertion member 4 and the vaginal insertion member 5 is not limited to this. The urethral insertion member 4 and the vaginal insertion member 5 may be configured so as not to be removable from each other.

Further, while the urethral insertion portion 41 in the puncture apparatus 1 is fixed to the support portion 40, the configuration of the urethral insertion portion 41 is not limited to this. The urethral insertion portion 41 may be configured so as to allow selection between a state in which the urethral insertion portion 41 is fixed to the support portion 40 and another state in which the urethral insertion portion 41 is slidably movable in the axial direction with respect to the support portion 40. In particular, the urethral insertion portion 41 may be configured such that, for example, if a screw provided on the support portion 40 is loosened, then the urethral insertion portion 41 is placed into a state in which it is slidably movable with respect to the support portion 40, but if the screw is tightened, then the urethral insertion portion 41 is placed into a state in which it is fixed to the support portion 40. With the configuration just described, since the length of the non-insertion portion 412 can be adjusted, the convenience in use of the insertion tool 6 can be improved. This similarly applies also to the vaginal insertion portion 51. Further, by moving the urethral insertion portion 41 forwardly or rearwardly in the urethra 1300, the balloon 42 attached to the bladder 1310 is contacted with the protrusion 11 of the vaginal insertion portion 51 through the living body tissue, by which the position of the balloon 42 in the bladder 1310 can be captured with relative certainty.

Further, while the components of the puncture apparatus 1 are fixed to the frame 2 such that the inclination angle θ2 is fixed, the configuration of the puncture apparatus 1 is not limited to this, and the inclination angle θ2 may be variable. Since this makes it possible to adjust the inclination angle θ2 in accordance with the patient, the convenience in use of the puncture apparatus 1 is improved.

Now, a method of using the puncture apparatus 1 is described. However, prior to the description of the method of use, the implant 9 which is used together with the puncture apparatus 1 is described.

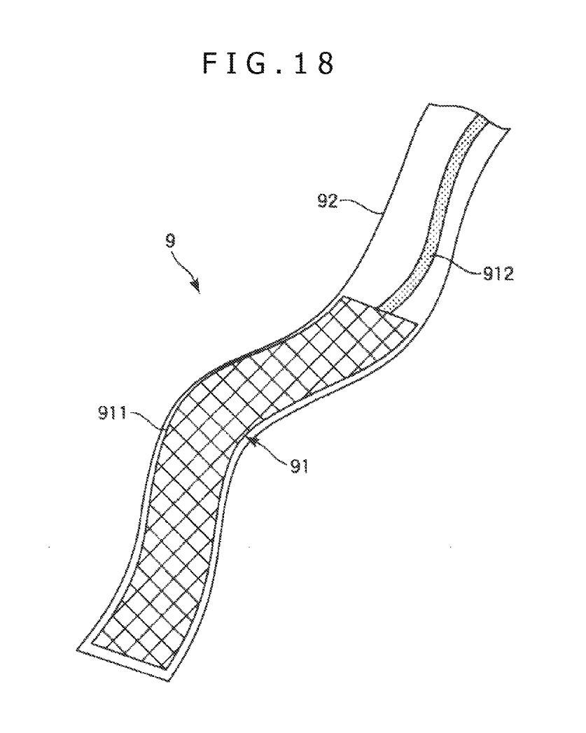

The implant (living body tissue-supporting indwelling article) 9 depicted in FIG. 18 is an implantable tool for treatment of urinary incontinence of women, namely, a tool for supporting the urethra, for example, a tool for supporting, when the urethra tends to move to the vaginal wall side, the urethra so as to restrict such a movement, in the manner of pulling the urethra in the direction for spacing away from the vaginal wall. As the implant 9, for example, an elongate article having flexibility can be used.

The implant 9 has the implant main body (belt-shaped elongate article) 91 and a packaging material 92 in the form of a bag for accommodating the implant main body 91 therein. The implant main body 91 has the main body portion 911 and a band 912 interlocked to one side of the main body portion 911. Since the implant 9 can include the packaging material 92, contamination of the implant main body 91 can be effectively prevented. Note that a guide wire, a cord, or a string may be used in place of the band 912.

The main body portion 911 has a form of a net and has a general shape of a belt. Note that the main body portion 911 can be configured, for example, from an article formed by crossing linear objects with each other into a braid, namely, a net-like braid. The linear objects may be those having a circular transverse sectional shape, or those having a flattened transverse sectional shape, namely, those of a belt-like shape.

The constituent materials of the main body portion 911, band 912 and packaging material 92 are not limited particularly, and for example, various resin materials and fiber materials having biocompatibility such as polypropylene, polyester or nylon.

Note that the implant 9 is not limited to an implant of the net-like shape only if it can demonstrate a similar effect. A pelvic treatment kit of the present disclosure is configured from such an implant 9 as just described and the sheath 30.

Now, an operation procedure (puncture method) of the puncture apparatus 1, namely, a procedure when the implant 9 is to be embedded into a living body is described.

First, the patient is set to a lithotomy position on an operating table, and the urethral insertion portion 41 of the urethral insertion member 4 is inserted into the urethra 1300 of the patient as depicted in FIG. 19. Thereupon, the insertion depth is confirmed from the ring member 46 disposed at a predetermined position in advance and the balloon 42 is disposed into the bladder 1310. The urethra 1300 is corrected to a predetermined shape by the urethral insertion portion 41 of the predetermined shape. In the case of the present embodiment, the urethra 1300 is corrected to a linear state by the linear urethral insertion portion 41.

Then, the balloon 42 is expanded, and the urine is drained from within the bladder 1310 through the drain hole 471 as occasion demands.

Then, the vaginal insertion portion 51 of the vaginal insertion member 5 is inserted into the vagina 1400 of the patient as depicted in FIG. 20A. Thereupon, the puncture position is confirmed from the marker 57, and the vaginal insertion portion 51 is inserted to an appropriate depth thereof.

Then, the urethral insertion member 4 is pulled to the proximal side. At this time, the urethral insertion member 4 is placed into a state depicted in FIG. 20A or into another state depicted in FIG. 20B.

In the state depicted in FIG. 20A, the balloon 42 attaches lightly to the bladder neck 1320 without excessively entering the urethra 1300 and oscillation upon the attachment is transmitted to the protrusion 11 through the bladder neck 1320 as aforementioned. Consequently, the operator can perceive the fact that the balloon 42 attaches to the bladder neck 1320 and therefore the puncture route M is in a state in which it is positioned on the urethral orifice side with respect to the bladder neck 1320, namely, in a state which is preferable for puncture.

On the other hand, in the state depicted in FIG. 20B, oscillation is not transmitted to the operator. Therefore, the operator can estimate that the balloon 42 excessively enters the urethra 1300, which is not a state suitable for puncture. In this case, the urethral insertion member 4 is pushed or pulled to establish the state of FIG. 20A.

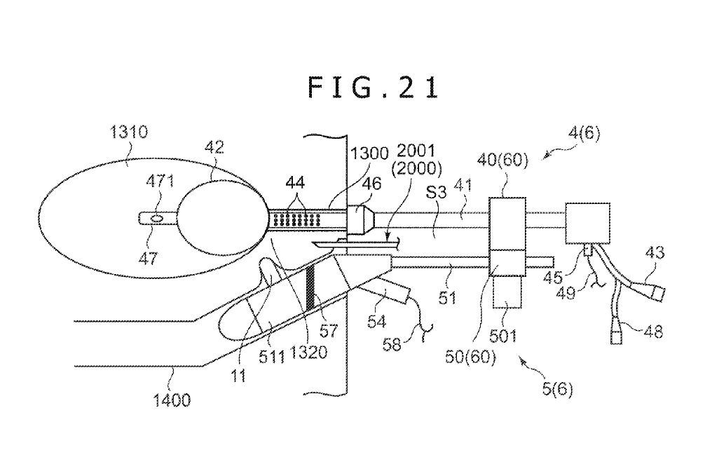

Then, the male screw 501 is operated to fix the support portions 40 and 50 as depicted in FIG. 21. Mounting of the insertion tool 6 on the patient is thereby completed. In this state, the non-insertion portions 412 and 512 are spaced apart from each other and besides the support unit 60 is spaced apart from the body surface between the urethral orifice and the vaginal orifice, and the body surface is exposed. In addition, where the insertion portion 511 and the vaginal front wall are spaced apart from each other and a gap (space) is formed between them, a space S3 in which an injector (injecting tool) 2000 is to puncture the living body tissue between the urethra 1300 and the vagina 1400 is formed from the body surface between the urethral orifice and the vaginal orifice. Note that the injector 2000 is filled with physiological saline solution or liquid for local anesthesia and has an injection needle 2001 at a distal end portion thereof.

Further, the ring member 46 of the urethral insertion member 4 is closely contacted with and fixed to the urethral orifice.

Then, suction devices are connected to the suction ports 45 and 54 and rendered operative to suck the urethra 1300 to the urethral insertion portion 41 and suck the vaginal front wall to the vaginal insertion portion 51. For example, if the urethra 1300 is sucked accurately to the urethral insertion portion 41, then since the suction holes 44 are closed up with the urethral wall, the suction from the suction port 45 is stopped or weakened. Similarly, if the vaginal front wall is sucked accurately to the vaginal insertion portion 51, then since the suction holes 59 are closed up with the vaginal wall, the suction from the suction port 54 is stopped or weakened. Therefore, the operator can confirm, from the degree of the suction (for example, from the magnitude of sound generated by the suction) from the suction ports 45 and 54, whether or not the urethra 1300 and the vaginal front wall are sucked accurately to the urethral insertion portion 41 and the vaginal insertion portion 51, respectively. Note that the insertion tool 6 may have a confirmation mechanism for mechanically confirming the suction state. Although the confirmation mechanism is not limited particularly if it allows confirmation of the suction state, the confirmation mechanism may be configured such that, for example, it can include a flow rate measuring unit (negative pressure gauge) for measuring the flow rate from the suction port 54 and a decision unit for deciding on the basis of a result of the measurement from the flow rate measuring unit whether or not the suction is performed accurately.

Thereafter, liquid dissection can be performed. In particular, the injection needle 2001 of the injector 2000 is caused to puncture the vaginal front wall 1410 from the proximal side through the space (space S3) between the insertion portion 511 and the vaginal front wall 1410 as depicted in FIG. 21. Then, physiological saline solution, liquid for local anesthesia is injected into the living body tissue between the urethra 1300 and the vagina 1400 (between the regions 51 and S2). By this, the living body tissue between the regions 51 and S2 is inflated, whereupon the urethra 1300 is pressed against the urethral insertion portion 41 and the vaginal front wall 1410 is pressed against the vaginal insertion portion 51.

Here, also during liquid dissection, the suction from the suction holes 44 and 59 is preferably continuously performed. If the urethra 1300 is pressed against the urethral insertion portion 41 by the liquid dissection, then since the urethra 1300 is sucked to the urethral insertion portion 41, the suction from the suction port 45 is stopped or weakened. Similarly, if the vaginal front wall 1410 is pressed against the vaginal insertion portion 51, then since the vaginal front wall 1410 is sucked to the vaginal insertion portion 51, the suction from the suction port 54 is stopped or weakened. Accordingly, the operator can confirm from the degree of the suction from the suction ports 45 and 54 whether or not the liquid dissection is accurately performed.

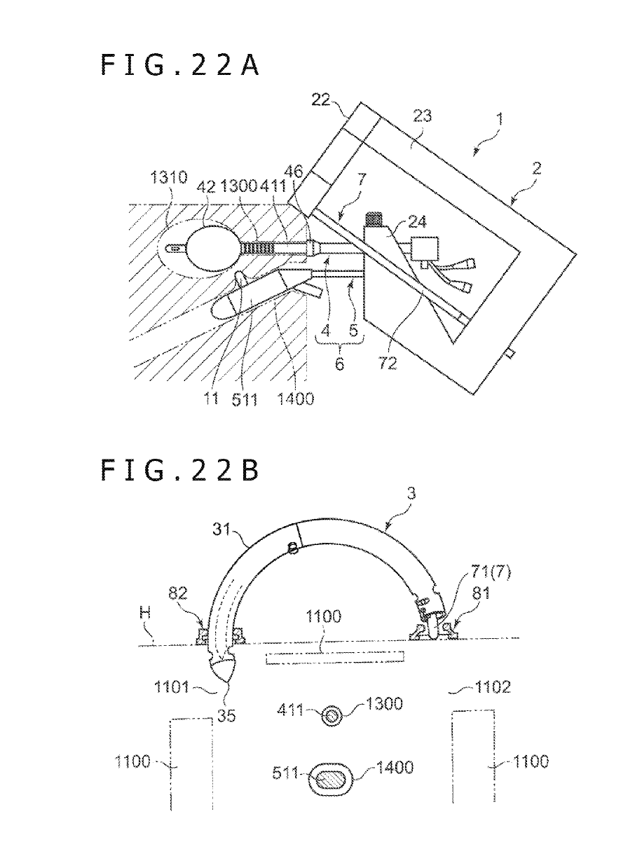

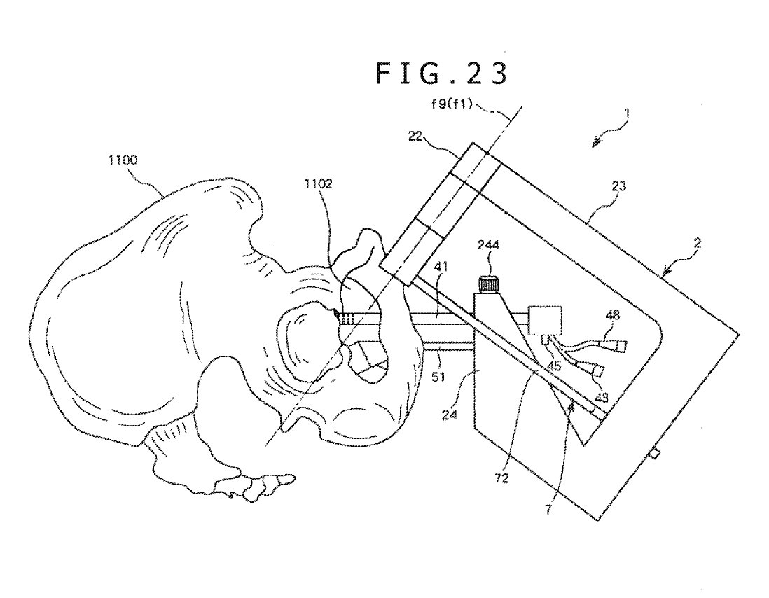

After the liquid dissection is performed to establish a state in which the urethra 1300 and the vaginal front wall 1410 are spaced apart from each other sufficiently, the frame 2 is fixed to the insertion tool 6 as depicted in FIG. 22A. Consequently, a state in which the puncture apparatus 1 is mounted on the patient is established. In this state, the positional relationship between the pelvis 1100 and the puncture apparatus 1 is such as depicted in FIG. 23.

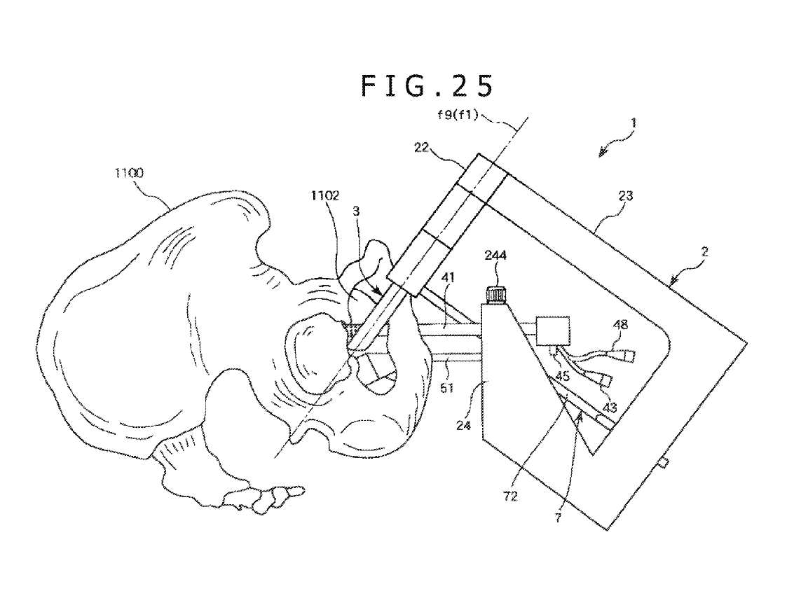

Then, for example, while the interlock portion 23 of the frame 2 is grasped by one hand, the interlock portion 72 of the operation member 7 is grasped by the other hand and the operation member 7 is turned in the counterclockwise direction as depicted in FIG. 24A. Consequently, the needle body 35 of the puncture member 3 punctures the body surface H at or in the proximity of the right side inguinal region of the patient (first region) from a direction different from that of the injection needle 2001 and enters the body. Then, the needle body 35 passes one 1101 of the obturator foramens, the region between the urethra 1300 and the vagina 1400 and the other obturator foramen 1102 in order, whereafter it projects to the outside of the body from the body surface H at or in the proximity of the left side inguinal region (second region) and finally is retracted into the guide portion 22 (refer to FIG. 25).

Consequently, the puncture member 3 is disposed in the living body and the anchors 81 and 82 are engaged with the main body 31 by the aforementioned principle. Therefore, the anchor 82 attaches to the body surface H to restrict further insertion of the proximal end portion of the main body 31 into the living body. In other words, the state in which the proximal end of the main body 31 is exposed to the outside of the living body can be secured.

Then, the operation member 7 is turned in the clockwise direction in FIG. 24A. Thereupon, although also the puncture member 3 tends to turn in the clockwise direction together with the operation member 7, since the anchor 81 attaches to the body surface H, such further turning motion (movement) is prevented. Therefore, while the state in which the distal end of the main body 31 is exposed to the outside of the living body is maintained, the insertion portion 71 is pulled out from the puncture member 3 and the living body. Thereafter, the puncture apparatus 1 (members other than the puncture member 3) is removed from the patient, and further, the needle body 35 is removed from the main body 31. Consequently, a state in which only the main body 31 is disposed in the living body as depicted in FIG. 24B can be established. In accordance with an exemplary embodiment, the main body 31 is disposed in the living body in a state in which the distal side opening and the proximal side opening thereof are exposed to the outside of the living body.

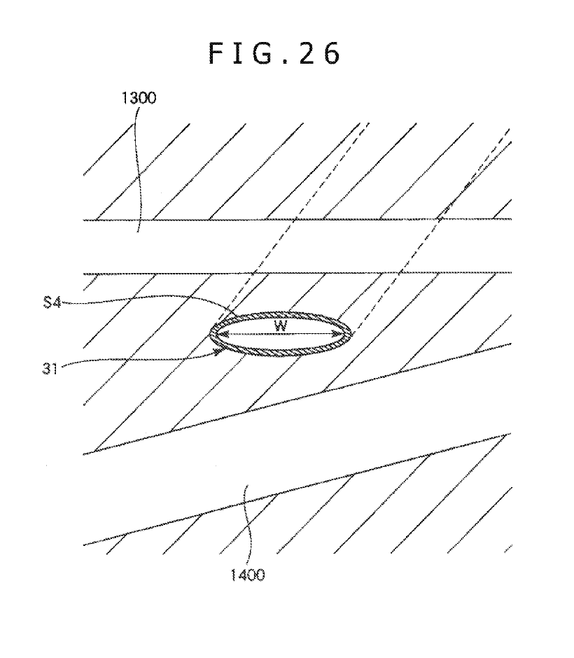

Then, the position of the main body 31 is adjusted as occasion demands. In particular, the main body 31 is displaced to the proximal side or the distal side so as to position the anchors 81 and 82 leftwardly and rightwardly symmetrically with respect to the living body. Consequently, the central portion S4 of the main body 31 can be positioned with a higher degree of accuracy between the urethra 1300 and the vagina 1400. In this state, the central portion S4 is disposed such that the widthwise direction (major axis J32 direction) W extends substantially in parallel to the urethra 1300 as depicted in FIG. 26. In other words, the urethra 1300 corrected by inserting the urethral insertion member 4 therein and the widthwise direction W of the central portion S4 are positioned substantially in parallel to each other.

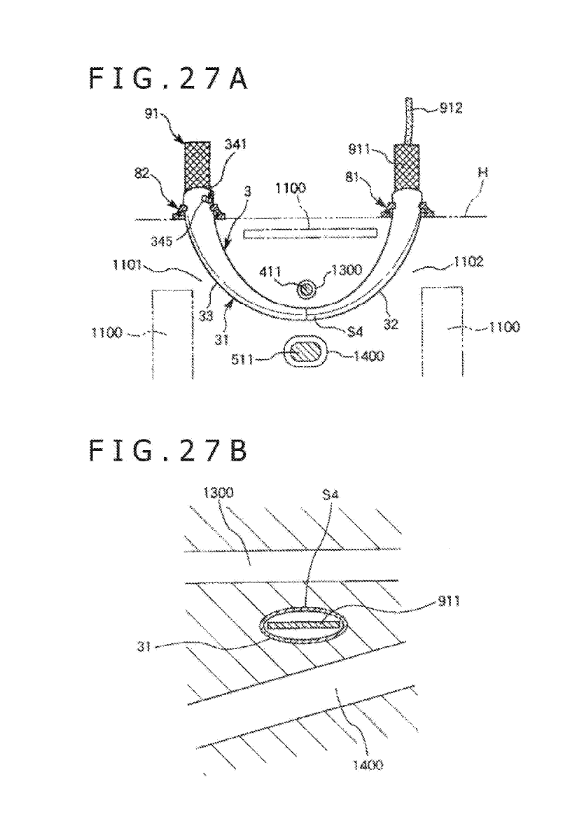

Thereafter, the implant main body 91 is inserted into the main body 31 while at the same time being taken out from the packaging material 92 to establish a state in which the band 912 projects from the proximal side opening and the distal side opening of the main body 31 as depicted in FIG. 27A. By leaving the implant main body 91 accommodated in the packaging material 92 till immediately before the implant main body 91 is disposed into the main body 31 in this manner, contamination of the implant main body 91 can be prevented. Note that, since the main body 31 has a flattened shape as aforementioned, the posture of the main body portion 911 follows the flattened shape. In particular, for example, as depicted in FIG. 27B, the main body portion 911 is disposed in the main body 31 such that the widthwise direction of the main body portion 911 coincides with the widthwise direction of the main body 31. As for the relationship to the urethra 1300, the implant main body 91 is disposed in parallel to the corrected urethra 1300.

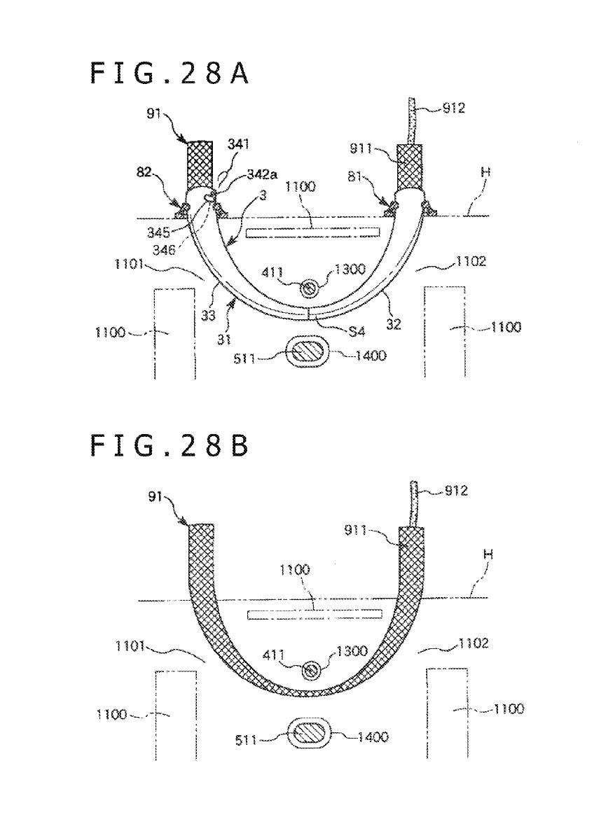

Then, the string 341 exposed from the exposure holes 345 and 346 is cut as depicted in FIG. 28A. Consequently, the main body 31 is placed into a state in which it can be divided into the distal end divisional piece 32 and the proximal end divisional piece 33. Note that, since the exposure holes 345 and 346 are positioned on the proximal side with respect to the anchor 82, they are exposed with certainty to the outside of the living body. Therefore, the string 341 can be readily cut.

Then, the suction of the urethra 1300 by the urethral insertion portion 41 and the suction of the vaginal front wall 1410 by the vaginal insertion portion 51 can be stopped. Consequently, the position and the shape of the urethra 1300 and the vagina 1400 return to their original states.

Then, the connection between the distal end divisional piece 32 and the proximal end divisional piece 33 is cancelled, and the distal end divisional piece 32 is pulled out toward the distal side from the living body and the proximal end divisional piece 33 is pulled out toward the proximal side from the living body. Thereupon, the distal end divisional piece 32 and the proximal end divisional piece 33 are moved substantially simultaneously in the opposite directions to each other and then are moved arcuately along the individual shapes. Consequently, the main body 31 is removed smoothly from the living body. As the distal end divisional piece 32 and the proximal end divisional piece 33 are removed from the living body in such a manner as aforementioned, the tissue in the surrounding portions separated by the main body 31 returns to its original state and is gradually contacted with the implant main body 91 from a central portion to the opposite end portions of the implant main body 91. As aforementioned, since the distal end divisional piece 32 and the proximal end divisional piece 33 are moved in their directions along the shapes of them and the main body 31 has an internal space sufficient to allow the implant main body 91 to move with low sliding resistance, the implant main body 91 can be indwelled in the state without being acted upon by unnecessary tension. Consequently, adjustment of the tension of the implant main body 91 is unnecessary. From the foregoing, the implant main body 91 is placed into a state in which it is embedded in the living body as depicted in FIG. 28B.

In the state in which the implant main body 91 is embedded in the living body, the main body portion 911 is disposed substantially in parallel to the urethra 1300 in the region between the urethra 1300 and the vagina 1400. Therefore, the urethra 1300 can be supported over an increased range by the implant main body 91.

In this manner, by dividing and removing the main body 31 from the living body, pulling out of the main body 31 from the living body can be readily performed. Further, even if the anchors 81 and 82 are not removed from the main body 31, the main body 31 can be pulled out from the living body, and consequently, the pulling out of the main body 31 can be readily performed. Further, with such a pulling out method as just described, the divisional pieces 32 and 33 being pulled out have little influence on the posture of the main body portion 911 in the region between the urethra 1300 and the vagina 1400.

Further, since the divisional pieces 32 and 33 are pulled out from within the living body in the state in which the urethral insertion member 4 is inserted in the urethra 1300, excessive tension can be prevented from acting upon the urethra 1300 by the implant main body 91 indwelled in the living body.

Then, the urethral insertion member 4 is pulled out from the urethra 1300 and the vaginal insertion member 5 is pulled out from the vagina 1400. After the urethral insertion member 4 is pulled out, the urethra 1300 returns to its shape of the natural state. However, since the main body portion 911 is embedded in the tissue, the parallel state between the urethra 1300 in the natural state and the main body portion 911 can be maintained.

Thereafter, unnecessary portions of the implant main body 91 can be cut away, thereby ending the manipulation.

As described above, with the puncture apparatus 1, prior to puncture by the puncture member 3, it can be detected that the puncture route M is in a state in which it interferes with the urethra 1300, and consequently, the puncture can be performed safely. Further, when the implant 9 is to be indwelled, this can be coped with, for example, only by low invasive manipulation such as puncture of the puncture member 3 and so forth, and there is no necessity to perform incision, which can be highly invasive. Therefore, the burden on the patient is relatively light and the safety of the patient is relatively high. Further, since the implant main body 91 can be embedded in parallel to the urethra 1300, the urethra 1300 can be supported over an increased area. Further, the living body can be punctured by the puncture member 3 avoiding the urethra 1300 and the vagina 1400, and such a situation that the puncture member 3 punctures the urethra 1300 or the vagina 1400 can be prevented. Further, the implant 9 can be prevented from being exposed to the inside of the vagina from a wound caused by incision of the vagina as in a conventional case in which the vagina is incised or that complications such as infection from the wound occur. Therefore, the puncture apparatus 1 is relatively safe and the implant 9 can be embedded with relative certainty.

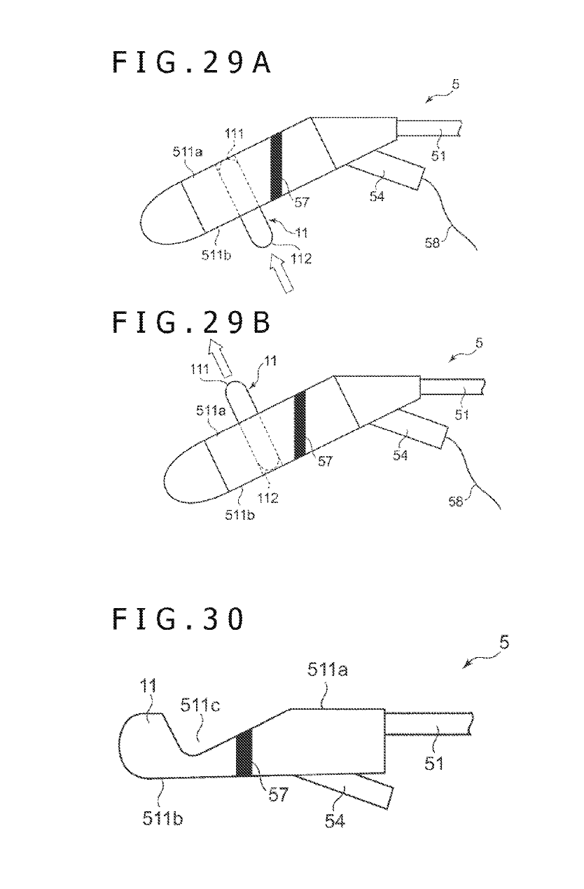

FIGS. 29A and 29B are lateral views illustrating an operation method of a vaginal insertion member possessed by a living body insertion device (second embodiment).

In the following, the second embodiment of a living body insertion tool and a suction method of the present disclosure are described with reference to FIGS. 29A and 29B. However, the description is given principally of differences of the second embodiment from the aforementioned first embodiment while description of like matters is omitted herein to avoid redundancy.

The present embodiment is similar to the first embodiment described hereinabove except that it is different in configuration of the vaginal insertion member.

As depicted in FIGS. 29A and 29B, in the present embodiment, the protrusion 11 of the vaginal insertion member 5 is configured from a piece of a plate and supported such that the apex 111 thereof can project from and retreat to the upper face 511a of the vaginal insertion portion 51. Consequently, the protrusion 11 can assume a first state in which it is retracted from the urethral insertion member 4 (refer to FIG. 29A) and a second state in which it is projected to the urethral insertion member 4 side, namely, to the upper side in FIG. 29A (refer to FIG. 29B).

When the vaginal insertion member 5 is inserted into the vagina 1400, the protrusion 11 is preferably placed into the first state. This makes it possible, upon insertion of the vaginal insertion member 5 into the vagina 1400, to prevent the protrusion 11 from making an obstacle. Consequently, the insertion into the vagina 1400 can be facilitated. In addition, where it is desirable to press the upper face 511a of the vaginal insertion portion 51 against the vaginal wall after the insertion, the protrusion 11 can be prevented from making an obstacle.

Further, the protrusion 11 in the first state can be caused to transit to the second state.

Note that, in the first state, a portion 112 on the opposite side to the apex 111 of the protrusion 11 projects from the lower face 511b of the vaginal insertion portion 51. By pressing the projecting portion 112, the protrusion 11 can be readily placed into the second state and with certainty.

FIG. 30 is a lateral view of a vaginal insertion member possessed by a living body insertion device (third embodiment).

In the following, the third embodiment of a living body insertion tool and a suction method of the present disclosure is described with reference to FIG. 30. However, the description is given principally of differences of the third embodiment from the aforementioned embodiments while description of like matters is omitted herein to avoid redundancy.

The present embodiment is similar to the first embodiment described hereinabove except that it is different in shape of the vaginal insertion member.

As depicted in FIG. 30, in the present embodiment, a recessed portion 511c is formed on the upper face 511a of the vaginal insertion portion 51. The puncture route M can pass so as to be spaced apart from the recessed portion 511c.

The recessed portion 511c has a wedge-shaped longitudinal sectional shape. Where such a recessed portion 511c as just described is formed, a portion on the distal side of the recessed portion 511c demonstrates a projecting state, and the projecting portion functions as the protrusion 11.

With such a configuration as just described, projection of the protrusion 11 from the upper face 511a can be reduced. Consequently, when the vaginal insertion member 5 is inserted into the vagina 1400, the protrusion 11 can be prevented from making an obstacle, and therefore, the insertion is facilitated.

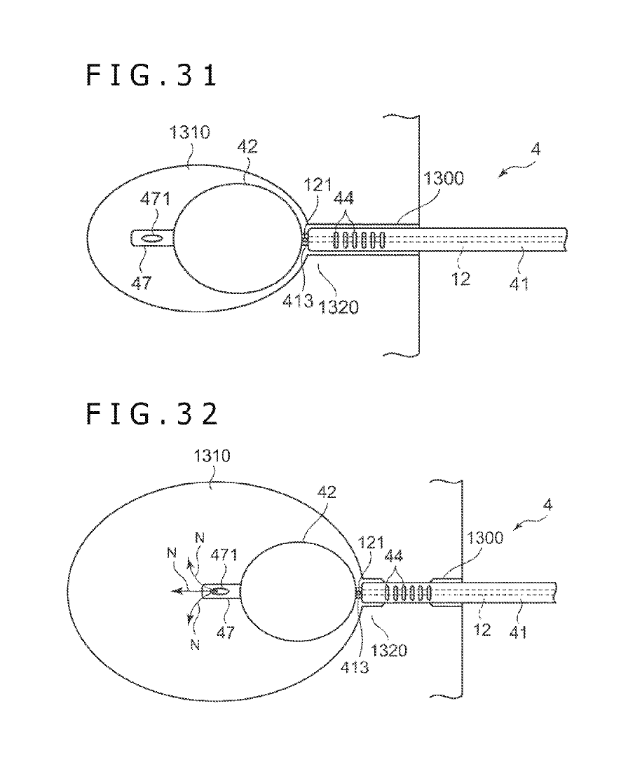

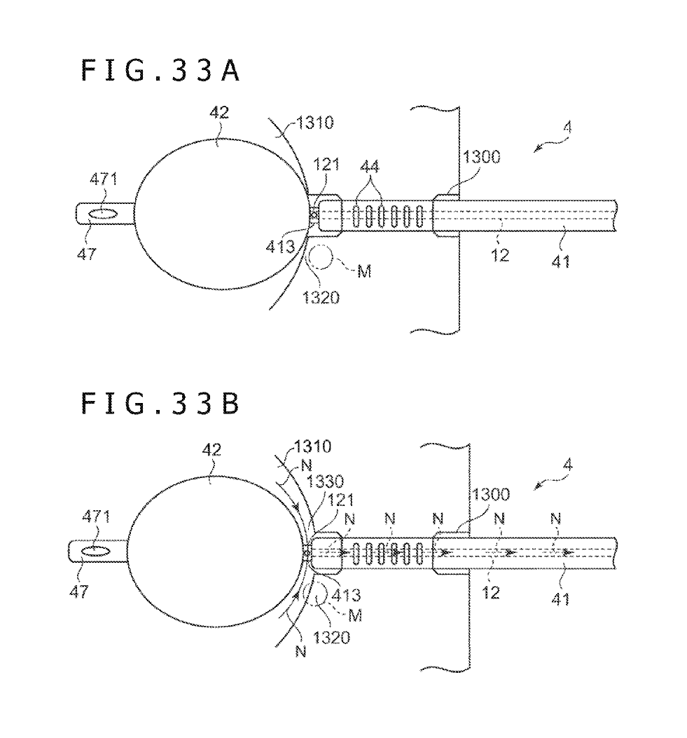

FIGS. 31 and 32 are views illustrating an operation procedure of a living body insertion device (fourth embodiment). FIGS. 33A and 33B illustrate the operation procedure of the living body insertion device (fourth embodiment), wherein FIG. 33A is a view when the operation is performed appropriately and FIG. 33B is a view when the operation is performed inappropriately.

In the following, the fourth embodiment of a living body insertion tool and a suction method of the present disclosure is described with reference to FIGS. 31 to 33B. However, the description is given principally of differences of the fourth embodiment from the aforementioned embodiments while description of like matters is omitted herein to avoid redundancy.

The present embodiment is similar to the first embodiment described hereinabove except that it is different in configuration of the urethral insertion member.

As depicted in FIGS. 31 to 33B, in the present embodiment, the urethral insertion member 4 has at least one detection lumen (flow path) 12 which functions as a position detection unit for detecting that the balloon 42 attaches to the bladder neck 1320. The detection lumen 12 is formed along a longitudinal direction of the urethral insertion portion 41 of the urethral insertion member 4. From a result of the detection (detection method), it can be decided whether or not a positioned state in which the balloon 42 attaches to and is positioned by the bladder neck 1320 is correct.

Further, the detection lumen 12 has an inlet port 121 into which liquid in the bladder 1310 can flow. The inlet port 121 is provided on an outer circumferential face of the urethral insertion portion 41 in the proximity of the proximal side with respect to the balloon 42 and is formed such that it is open sidewardly in the bladder 1310 in a state in which the balloon 42 is inserted and disposed in the bladder 1310. Further, the inlet port 121 is formed at a position between the balloon 42 and the suction holes 44. Further, while the inlet port 121 is disposed so as to open in the bladder 1310, the opening of the inlet port 121 is not limited to this, and for example, the inlet port 121 may be open to both of the inside of the bladder 1310 and the urethra 1300 or may be open to the urethra 1300.

Note that, where a plurality of inlet ports 121 are formed, they are preferably disposed, for example, at equal distances along a circumferential direction of the urethral insertion portion 41.

As aforementioned, it sometimes occurs that, in a state in which the urethral insertion member 4 is inserted in the urethra 1300, the balloon 42 is expanded and the urethral insertion member 4 is pulled in a direction toward the proximal end trying to attach the balloon 42 to the bladder neck 1320 in order to position the urethral insertion member 4. If this pulling is insufficient, then a state in which the balloon 42 does not attach to and is not positioned by the bladder neck 1320 is established as depicted in FIG. 33B. Also in the state depicted in FIG. 33B, the puncture route M and the urethra 1300 (bladder neck 1320) are sometimes in a state in which they interfere with each other. This is not preferable to treatment of urinary incontinence of women. Further, such a state as just described is less likely to be recognized by the operator.

Therefore, it can be detected from the detection lumen 12 whether or not the expanded balloon 42 of the urethral insertion member 4 attaches to the bladder neck 1320 in such a manner as to cover the bladder neck 1320 and is thereby positioned. In the following, this is described.

First, the urethral insertion member 4 is inserted into the urethra 1300 and the balloon 42 is expanded in the bladder 1310 as depicted in FIG. 31. Thereafter, the urine in the bladder 1310 is drained (flows out) through the urine drainage portion 47.

Then, the urethral insertion member 4 is pulled to the proximal side and the urethral wall is sucked by the suction holes 44 and then water N is supplied through the urine drainage portion 47 as depicted in FIG. 32. Consequently, the inside of the bladder 1310 can be filled with the water N once.

Then, if the pulling of the urethral insertion member 4 is appropriate, then the balloon 42 attaches to the bladder neck 1320 as depicted in FIG. 33A. On the other hand, if the pulling is not appropriate, namely, if the pulling is insufficient, then the balloon 42 does not attach to the bladder neck 1320 and a gap 1330 is formed between the balloon 42 and the bladder neck 1320 as depicted in FIG. 33B.

Then, if the positioned state is correct, then the balloon 42 attaches to the bladder neck 1320 and seals the periphery of the bladder neck 1320 as depicted in FIG. 33A. Therefore, the water N is inhibited from entering the inlet port 121, namely, the water N does not flow down along the detection lumen 12. Further, if the positioned state is not good, then the water N will flow into the inlet port 121 through the gap 1330 as depicted in FIG. 33B. Consequently, the water N flows down along the detection lumen 12. The operator can confirm the water N flowing down along the detection lumen 12, namely, can confirm flashback.

In this manner, with regard to the urethral insertion member 4, whether or not flashback is observed can be confirmed, namely, a result of detection by the detection lumen 12. Consequently, the operator can decide and understand that the positioned state is correct and the puncture route M is positioned on the urethral orifice side with respect to the bladder neck 1320.

Then, if puncture (a puncturing method) by the puncture member 3 is performed, then the puncture route M passes a targeted region of the living body tissue. Therefore, safe puncture can be performed with relative certainty.

A portion of the urethral insertion portion 41 at which the inlet port 121 is disposed is formed as a reduced diameter portion 413 which has a thickness smaller than that of a portion of the urethral insertion portion 41 at which the suction holes 44 are disposed. If suction of the urethral wall is performed by the suction holes 44, then the urethral wall is pulled to the urethral insertion portion 41 side and is closely contacted with the outer circumferential face of the urethral insertion portion 41. Although the pulled urethral wall sometimes reaches the inlet port 121, it can be prevented at the reduced diameter portion 413 from closely contacting with the urethral insertion portion 41. Consequently, the inlet port 121 is prevented with relative certainty from being closed up with the urethral wall, and therefore, whether or not there exists flashback can be confirmed with relative certainty.

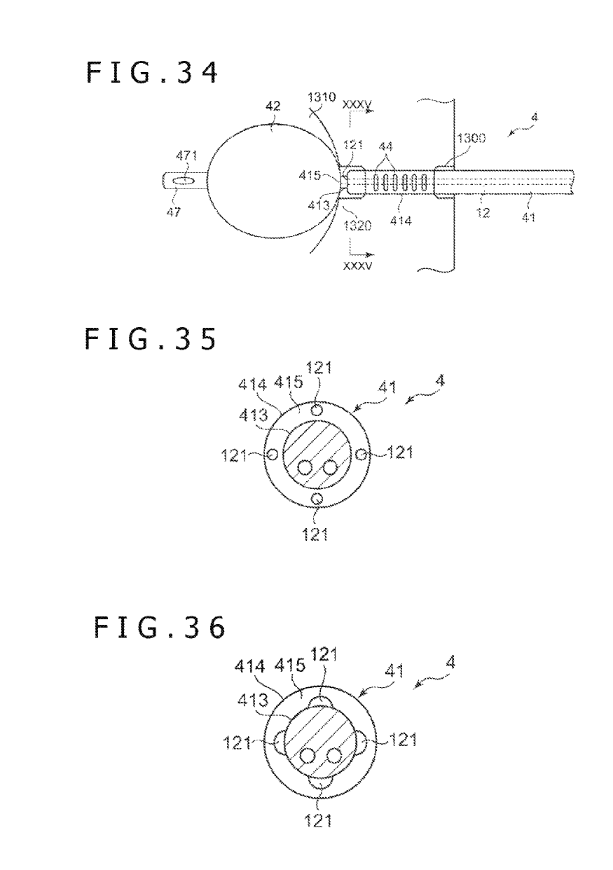

FIG. 34 is a lateral view of a urethral insertion member possessed by a living body insertion device (fifth embodiment). FIG. 35 is a sectional view taken along line XXXV-XXXV of FIG. 34.

In the following, the fifth embodiment of a living body insertion tool and a suction method of the present disclosure is described with reference to FIGS. 34 and 35. However, the description is given principally of differences of the fifth embodiment from the aforementioned embodiments while description of like matters are omitted herein to avoid redundancy.

The present embodiment is similar to the fourth embodiment described hereinabove except that it is different in configuration of the urethral insertion member.

As depicted in FIG. 34, in the present embodiment, the inlet port 121 is provided at a boundary region 415 between the reduced diameter portion 413 of the urethral insertion portion 41 and an increased diameter portion 414 on the proximal side with respect to the reduced diameter portion 413 and is directed toward the distal end. Since the inlet port 121 is disposed in this manner, it is prevented with a higher degree of certainty that the inlet port 121 is closed up with the sucked urethral wall. Therefore, whether or not a flashback exists can be confirmed with a relatively higher degree of certainty.

FIG. 36 is a transverse sectional view of a urethral insertion member possessed by a living body insertion device (sixth embodiment).

In the following, the sixth embodiment of a living body insertion tool and a suction method of the present disclosure is described with reference to FIG. 36. However, the description is given principally of differences of the sixth embodiment from the aforementioned embodiments while description of like matters are omitted herein to avoid redundancy.

The present embodiment is similar to the fifth embodiment described hereinabove except that it is different in configuration of the urethral insertion member.

As depicted in FIG. 36, in the present embodiment, a plurality of inlet ports 121 are unevenly distributed to the reduced diameter portion 413 side. Where the urethral insertion portion 41 is structured such that the reduced diameter portion 413 is inserted in and assembled to the increased diameter portion 414, if the inlet ports 121 are disposed in an unevenly distributed manner on the reduced diameter portion 413 side, then formation of the inlet ports 121 can be performed readily. Further, the size of the inlet ports 121 can be set greater than that in the fifth embodiment.

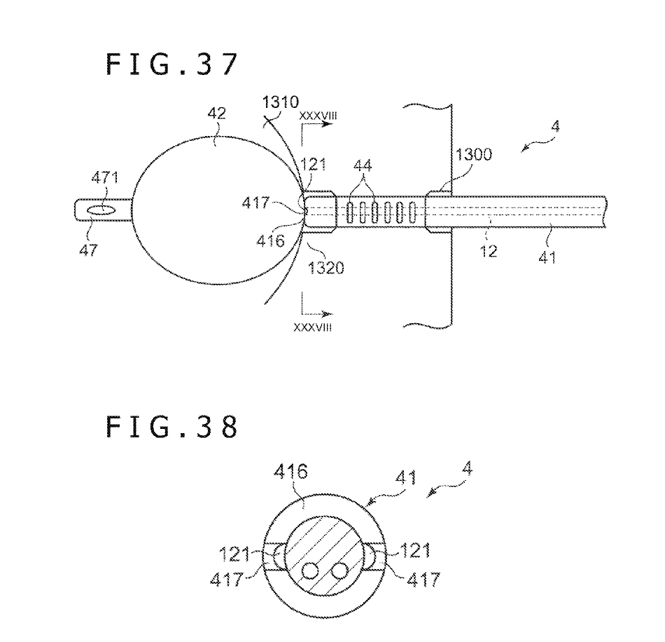

FIG. 37 is a lateral view of a urethral insertion member possessed by a living body insertion device (seventh embodiment). FIG. 38 is a sectional view taken along line XXXVIII-XXXVIII of FIG. 37.

In the following, the seventh embodiment of a living body insertion tool and a suction method of the present disclosure is described with reference to FIGS. 37 and 38. However, the description is given principally of differences of the seventh embodiment from the aforementioned embodiments while description of like matters are omitted herein to avoid redundancy.

The present embodiment is similar to the fourth embodiment described hereinabove except that it is different in configuration of the urethral insertion member.

As depicted in FIGS. 37 and 38, in the present embodiment, the inlet ports 121 are provided at a boundary portion 416 of the urethral insertion portion 41 from the balloon 42 and is directed toward the distal end. Further, grooves 417 are formed on the urethral insertion portion 41 such that they extend from an outer circumferential face of the urethral insertion portion 41 to the inlet ports 121. Consequently, when the positioned state is not appropriate, liquid in the bladder 1310 can flow to the inlet ports 121 through the grooves 417.

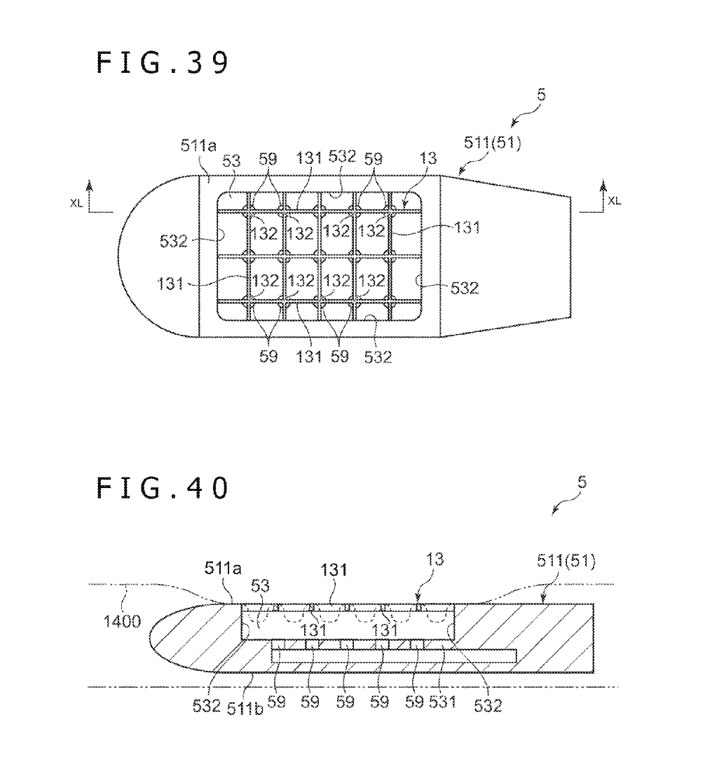

FIG. 39 is a plan view of a vaginal insertion member possessed by a living body insertion device (eighth embodiment). FIG. 40 is a sectional view taken along line XL-XL of FIG. 39.

In the following, the eighth embodiment of a living body insertion tool and a suction method of the present disclosure is described with reference to FIGS. 39 and 40. However, the description is given principally of differences of the eighth embodiment from the aforementioned embodiments while description of like matters are omitted herein to avoid redundancy.

The present embodiment is similar to the first embodiment described hereinabove except that it is different in configuration of the vaginal insertion member.

As depicted in FIGS. 39 and 40, in the present embodiment, the vaginal insertion member 5 has a single recessed portion 53 formed in a recessed shape on the upper face 511a which can attach to a living body tissue between the urethra 1300 and the vagina 1400, namely, to the vaginal wall. A plurality of suction holes 59 are disposed in rows and columns on the bottom 531 of the recessed portion 53.

As depicted in FIG. 40, the recessed portion 53 of the vaginal insertion member 5 has provided thereon a blocking portion 13 which blocks, in a state in which the vaginal wall is sucked, the vaginal wall from reaching the suction holes 59. The blocking portion 13 is configured from a plurality of linear members 131 having a linear shape at least at part thereof. The linear members 131 cross with each other to form a grating shape. Each linear member is provided so as to extend between, namely, bridge, opposing ones of side walls 532 provided uprightly from the bottom 531 of the recessed portion 53.

Note that the disposition location of the blocking portion 13 with respect to the recessed portion 53 is preferably set to a place as high as possible, namely, to a position spaced apart by a distance as large as possible from the bottom 531 of the recessed portion 53.

Further, the distance between the linear members 131 in the leftward and rightward direction and the distance between the linear members 131 in the upward and downward direction in FIG. 39 are preferably, for example, equal to or greater than, for example, 5 mm.

Further, the distance between the linear members 131 in the leftward and rightward direction and the distance between the linear members 131 in the upward and downward direction may be equal to each other or may be different from each other. Where the distances are different from each other, preferably the latter is greater than the former.

Further, while the blocking portion 13 in the present embodiment is configured such that a plurality of linear members 131 cross with each other to form a grating, the blocking portion 13 is not limited to this and may be configured such that a plurality of linear members 131 are disposed in parallel in one direction.

Further, the transverse sectional shape of the linear members 131 is not limited particularly and may be, for example, a polygonal shape, a circular shape, or an elliptical shape.

Further, the wire diameter of the linear members 131 may be smaller than, equal to, or greater than the opening diameter of the suction holes 59.

Further, the constituent material of the linear members 131 is not limited particularly, and for example, various metal materials and various resin materials can be used.

Incidentally, the suction force to the vaginal wall increases in proportion to the area over which the vaginal wall is actually sucked (hereinafter referred to as “suction area”). Here, the suction area where the vaginal wall enters the bottom 531 of the recessed portion 53 and directly contacts with the suction holes 59 to close up the suction holes 59 and the suction area where the vaginal wall enters midway of the recessed portion 53 but does not close up the suction holes 59 are compared with each other. The suction area in the former case is much smaller than the suction area in the latter case. Therefore, the suction force to the vaginal wall is lower in the former case than in the latter case. Consequently, the suction force to the vaginal wall may be so insufficient that the vaginal wall is spaced apart, namely, dislocated, from the upper face 511a of the vaginal insertion member 5.

Therefore, in the present embodiment, even in the suction state, such a situation that the vaginal wall closes up the suction holes 59 can be prevented with certainty by the blocking portion 13 as depicted in FIG. 40. This (suction method) can prevent drop of the suction force with relative certainty and maintain the suction state. Therefore, the vaginal wall can be prevented from being dislocated from the upper face 511a of the vaginal insertion member 5.

Further, when the vaginal wall is blocked from reaching the suction holes 59, namely, from contacting with the suction holes 59, by the blocking portion 13 as depicted in FIG. 40, the vaginal wall sinks into gaps between adjacent ones of the linear members 131. If the vaginal insertion member 5 is moved forwardly and rearwardly in this state, then also the vaginal wall follows up the moving direction of the vaginal insertion member 5. The positional adjustment of the puncture route M can be thereby performed.

As depicted in FIG. 39, when the upper face 511a is viewed in plan, the suction holes 59 overlap with crossing portions 132 of the linear members 131. In other words, the linear members 131 overlap with the suction direction of the suction holes 59. By such a positional relationship as just described, even if the sucked vaginal wall enters to the proximity of the bottom 531 of the recessed portion 53, the suction holes 59 can be prevented with relative certainty from being closed up with the vaginal wall.

Note that the configuration of the blocking portion 13 can be applied not only to the vaginal insertion member 5 but also to the urethral insertion member 4.

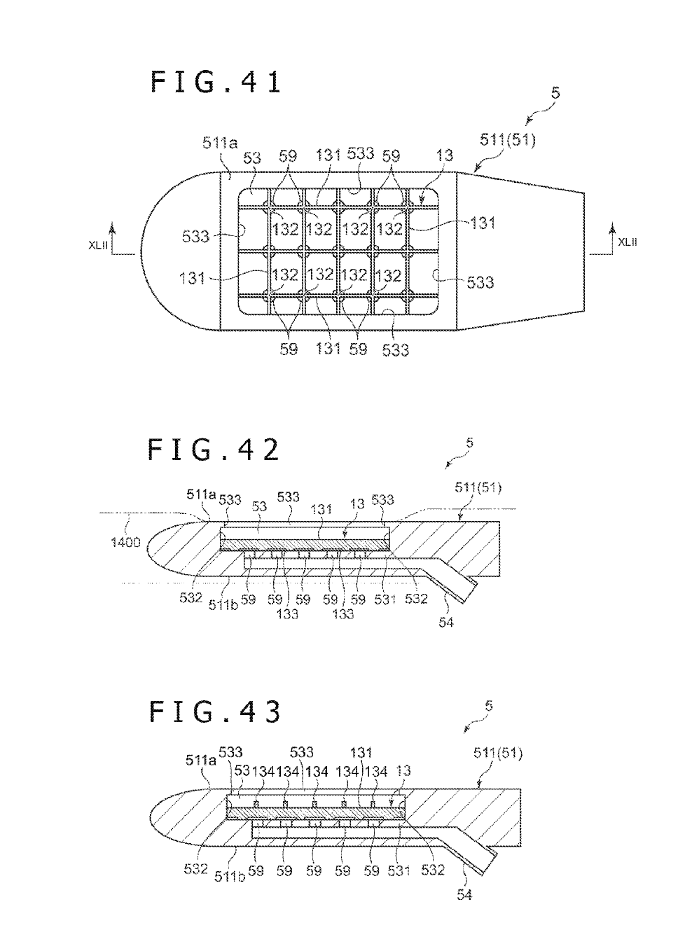

FIG. 41 is a plan view of a vaginal insertion member possessed by a living body insertion device (ninth embodiment). FIG. 42 is a sectional view taken along line XLII-XLII of FIG. 41.

In the following, the ninth embodiment of a living body insertion tool and a suction method of the present disclosure is described with reference to FIGS. 41 and 42. However, the description is given principally of differences of the ninth embodiment from the aforementioned embodiments while description of like matters are omitted herein to avoid redundancy.

The present embodiment is similar to the eighth embodiment described hereinabove except that it is different in configuration of the vaginal insertion member.

As depicted in FIGS. 41 and 42, in the present embodiment, the linear members 131 configuring the blocking portion 13 have a belt shape and are disposed such that the widthwise direction thereof coincides with the depthwise direction of the recessed portion 53. In other words, the bottom of the linear members 131 is disposed such that it coincides with the bottom 531 of the recessed portion 53.

Further, as depicted in FIG. 42, the blocking portion 13 is supported by the bottom 531 of the recessed portion 53 with spacers 133 interposed therebetween. Consequently, a gap is provided between the blocking portion 13 and the bottom 531 and communicates the blocking portion 13 and the bottom 531 with each other. Where the blocking portion 13 is disposed in the proximity of the bottom 531 of the recessed portion 53 in this manner, the linear members 131 preferably have a form of a belt whose widthwise direction coincides with the depthwise direction of the recessed portion 53 as aforementioned. The sucked vaginal wall can be thereby blocked from reaching the suction holes 59.

Further, where the blocking portion 13 is disposed in the proximity of the bottom 531 of the recessed portion 53, the vaginal wall enters to the deeper side of the recessed portion 53 than that in the eighth embodiment described hereinabove. Therefore, the side walls 532 of the recessed portion 53 preferably have protrusions 533 formed in a projecting manner thereon. The protrusions 533 can bite into the vaginal wall, which enters to the deep side of the recessed portion 53. Then, by moving the vaginal insertion member 5 forwardly and rearwardly in this state, also the vaginal wall follows up in the moving direction of the vaginal insertion member 5. Consequently, positional adjustment of the puncture route M can be performed.

FIG. 43 is a longitudinal sectional view of a vaginal insertion member possessed by a living body insertion device (tenth embodiment).

In the following, the tenth embodiment of a living body insertion tool and a suction method of the present disclosure is described with reference to FIG. 43. However, the description is given principally of differences of the tenth embodiment from the aforementioned embodiments while description of like matters are omitted herein to avoid redundancy.

The present embodiment is similar to the ninth embodiment described hereinabove except that it is different in configuration of the vaginal insertion member.

As depicted in FIG. 43, in the present embodiment, at least one of the linear members 131 which configure the blocking portion 13 has a protrusion 134 formed thereon such that it projects to the opposite side to the bottom 531, namely, to the upper side. Note that the protrusion 134 preferably extends in the deeper side with respect the plane of FIG. 43. Such a protrusion 134 as described above can bite into the vaginal wall, which enters the deep side of the recessed portion 53, together with the protrusions 533 of the recessed portion 53. Then, by moving the vaginal insertion member 5 forwardly and rearwardly in this state, also the vaginal wall follows up in the moving direction of the vaginal insertion member 5 with relative certainty. Consequently, positional adjustment of the puncture route M can be performed with relative certainty.

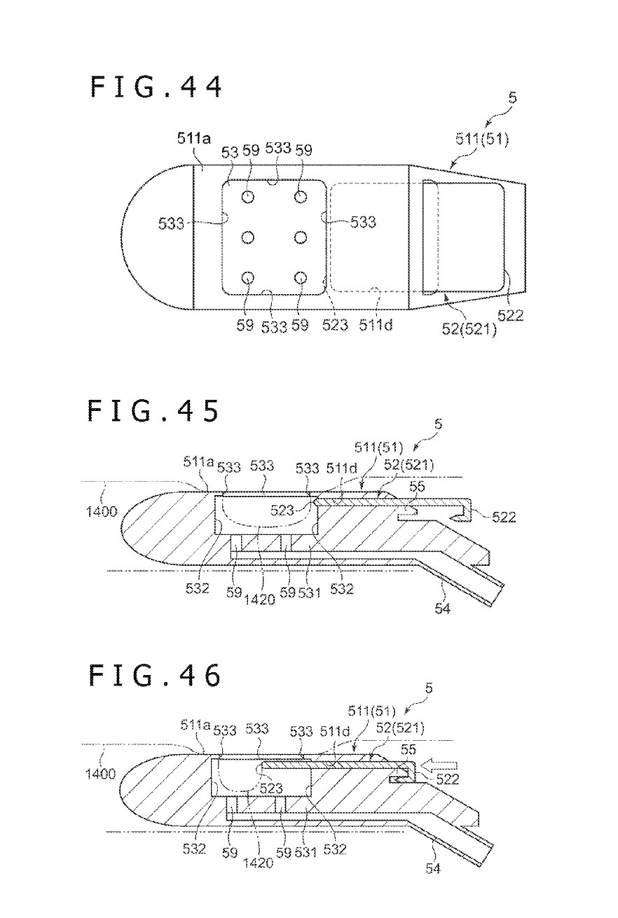

FIG. 44 is a plan view of a vaginal insertion member possessed by a living body insertion device (eleventh embodiment). FIGS. 45 and 46 are views illustrating an operation procedure of the vaginal insertion member depicted in FIG. 44.

In the following, the eleventh embodiment of a living body insertion tool and a suction method of the present disclosure is described with reference to FIGS. 44 to 46. However, the description is given principally of differences of the eleventh embodiment from the aforementioned embodiments while description of like matters are omitted herein to avoid redundancy.

The present embodiment is similar to the first embodiment described hereinabove except that it is different in configuration of the vaginal insertion member.

As depicted in FIGS. 44 to 46, in the present embodiment, the vaginal insertion member 5 has a holding portion 52. In the suction state, a suction target portion 1420 enters the recessed portion 53. The holding portion 52 is an element for holding the suction target portion 1420, which enters the recessed portion 53.

The holding portion 52 is configured from a sandwiching piece 521 supported for movement in a planar direction of the upper face 511a, namely, forwardly and rearwardly in the insertion direction of the vaginal insertion member 5, on the inner side of the recessed portion 53. The sandwiching piece 521 is configured from a piece of a plate and has a bent portion 522 which is bent, at a proximal end portion thereof, for example, twice in one direction, namely, in a “U shape.” This bent portion 522 functions as a pressing portion which is pressed when the sandwiching piece 521 is operated to move. A portion of the sandwiching piece 521 on the opposite side to the bent portion 522 functions as a sandwiching portion 523 for sandwiching the suction target portion 1420 at the destination of the movement of the sandwiching piece 521.

Such a sandwiching piece 521 as described above is inserted in a groove 511d formed on the insertion portion 511 of the vaginal insertion portion 51. The groove 511d is formed in parallel to the upper face 511a and is open just below the protrusions 533 of the recessed portion 53. Consequently, the sandwiching piece 521 can project into the recessed portion 53. The projection amount of the sandwiching piece 521 is adjusted by movement of the sandwiching piece 521. In a first state depicted in FIG. 45, the sandwiching piece 521 is retracted with respect to the recessed portion 53, and in a second state depicted in FIG. 46, the sandwiching piece 521 projects most from the recessed portion 53. Note that the sandwiching piece 521 can naturally assume an intermediate state (third state) between the first state and the second state during the movement.

As depicted in FIG. 45, before the suction is started, the sandwiching piece 521 is in the first state. In this state, the vaginal wall is partly sucked toward the inside of the recessed portion 53 by the sucking force and the suction target portion 1420 thereof enters the inside of the recessed portion 53.

Thereafter, if the bent portion 522 is pressed in a direction toward the distal end as depicted in FIG. 46, then the sandwiching piece 521 is placed into the second state. In this state, the suction target portion 1420 is sandwiched between the sandwiching portion 523 and the protrusion 533 of the side wall 532 positioned forwardly in the direction of movement of the sandwiching piece 521.

The suction force is sometimes dropped, for example, by a failure of the suction device connected to the suction port 54 of the vaginal insertion member 5 to allow the suction state to the suction target portion 1420 to be cancelled. Further, the surface of the vagina and the attachment face of the suction portion can be displaced from each other, resulting in disengagement of the vaginal insertion member 5. However, even if the suction state is lost, force similar to the force to establish the suction state, namely, the pulling force for pulling the suction target portion 1420 to the recessed portion 53 (vaginal insertion member 5) side, can be continued to act upon the suction target portion 1420 with certainty by the sandwiching piece 521 (suction method) which is in the second state. Then, a succeeding puncture operation can be rapidly entered.

Note that the constituent material of the sandwiching piece 521 is not limited particularly, and for example, various resin materials and various metal materials can be used.

Further, the vaginal insertion member 5 has a movement limit defining portion 55 for defining the movement limit for the sandwiching piece 521. As depicted in FIG. 46, the bent portion 522 of the sandwiching piece 521 which is in the second state can attach to the movement limit defining portion 55, and consequently, the limit of movement in a direction toward the distal end is defined. Therefore, excessive force can be prevented from acting upon the suction target portion 1420.

Further, the movement limit defining portion 55 can be inserted into and engaged with the bent portion 522 bent in a “U” shape as aforementioned. By this engagement, the second state can be maintained. Therefore, even if the pressing force upon the bent portion 522 of the sandwiching piece 521 is cancelled, the sandwiching piece 521 can continue to hold the suction target portion 1420.

Note that the configuration of the holding portion 52 can be applied not only to the vaginal insertion member 5 but also to the urethral insertion member 4.

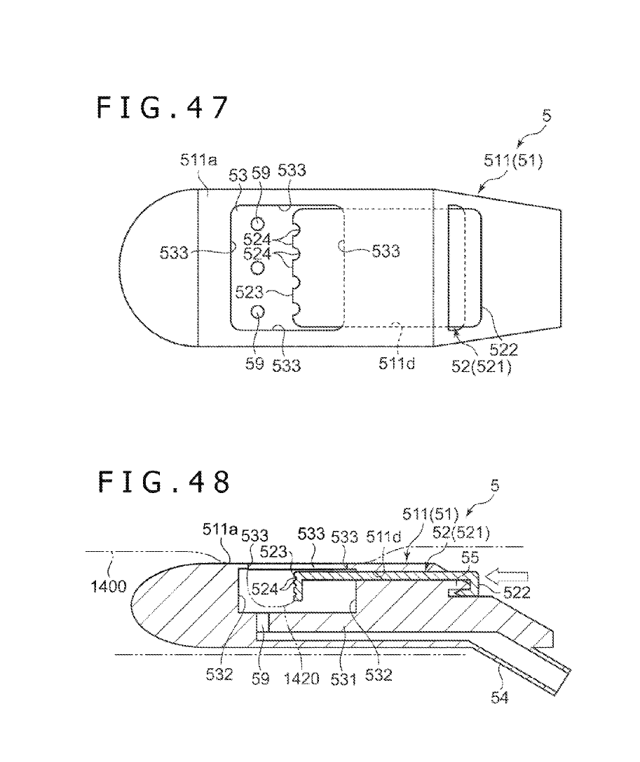

FIG. 47 is a plan view of a vaginal insertion member possessed by a living body insertion device (twelfth embodiment). FIG. 48 is a longitudinal sectional view of the vaginal insertion member depicted in FIG. 47.

In the following, the twelfth embodiment of a living body insertion tool and a suction method of the present disclosure is described with reference to FIGS. 47 and 48. However, the description is given principally of differences of the twelfth embodiment from the aforementioned embodiments while description of like matters are omitted herein to avoid redundancy.

The present embodiment is similar to the eleventh embodiment described hereinabove except that it is different in configuration of the vaginal insertion member.

As depicted in FIGS. 47 and 48, in the present embodiment, the sandwiching piece 521 is bent once at the sandwiching portion 523, and a plurality of projections and recesses 524 are formed on the bent sandwiching portion 523. Consequently, the projections and recesses 524 can bite into the suction target portion 1420 sandwiched in the second state, and the suction target portion 1420 can be prevented with relative certainty from coming off inadvertently from the recessed portion 53.

As depicted in FIG. 47, the suction holes 59 are disposed at positions at which they do not overlap with the sandwiching piece 521 which is at the limit of movement, namely, with the sandwiching piece 521 which is in the second state, when the upper face 511a is viewed in plan. Consequently, the suction holes 59 are not covered with the sandwiching piece 521 in the suction direction thereof. On the other hand, at positions at which the suction holes 59 overlap with the sandwiching piece 521 which is in the second state, in the eleventh embodiment, some of the suction holes 59 that are positioned on the proximal side are disposed.

As described hereinabove in connection with the eleventh embodiment, the suction target portion 1420 pulled in the recessed portion 53 in the first state is pressed in a direction toward the distal end in the second state. In this case, the suction target portion 1420 is released somewhat from the suction force by the suction holes 59 positioned on the proximal side. Therefore, the suction holes 59 positioned on the proximal side may be omitted. Consequently, simplification of the structure of the vaginal insertion member 5 can be anticipated.

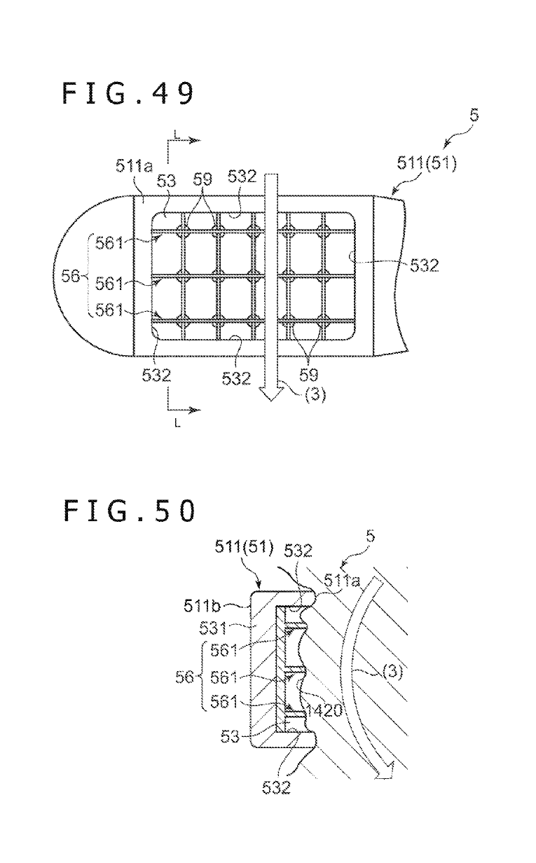

FIG. 49 is a plan view of a vaginal insertion member possessed by a living body insertion device (thirteenth embodiment). FIG. 50 is a sectional view taken along line L-L of FIG. 49.

In the following, the thirteenth embodiment of a living body insertion tool and a suction method of the present disclosure is described with reference to FIGS. 49 and 50. However, the description is given principally of differences of the thirteenth embodiment from the aforementioned embodiments while description of like matters are omitted herein to avoid redundancy.

The present embodiment is similar to the first embodiment described hereinabove except that it is different in configuration of the vaginal insertion member.

As depicted in FIGS. 49 and 50, in the present embodiment, the vaginal insertion member 5 has a positional displacement preventing portion 56. The positional displacement preventing portion 56 is an element for preventing or suppressing the vaginal wall from being displaced, for example, namely from moving, when the vaginal wall is punctured by the puncture member 3 in the suction state, in the puncture direction of the puncture member 3 (in the direction indicated by a blank arrow mark in FIGS. 49 and 50) with respect to the upper face 511a.

Such a positional displacement preventing portion 56 as just described has a plurality of (in the present embodiment, three) ribs (first protrusions) 561 formed to project from the bottom 531 of the recessed portion 53. One of the three ribs 561 is disposed at a central location in the widthwise direction of the vaginal insertion member 5 while the remaining two ribs 561 are disposed at equal distances from the centrally located rib 561.

The ribs 561 extend in the longitudinal direction of the vaginal insertion member 5, namely, in a direction orthogonal to the puncture direction of the puncture member 3. The ribs 561, for example, have an equal height and may be equal in height to the side walls 532 of the recessed portion 53. However, preferably, for example, the ribs 561 are lower than the side walls 532.

Note that, although the number of ribs 561 is, in the present embodiment, a plural number, the number is not limited to this and may be, for example, one.

When puncture by the puncture member 3 is to be performed, a suction state is established to raise the closeness of contact between the upper face 511a of the vaginal insertion member 5 and the vaginal wall and then the puncture is performed in this state. By the puncture, also the vaginal wall is pulled in the puncture direction. Then, if the pulling force is excessively high, then the vaginal wall will be displaced from the upper face 511a of the vaginal insertion member 5 and released from the suction state.

However, as depicted in FIG. 50, in the vaginal insertion member 5, the ribs 561 sink into the suction target portion 1420 entering the recessed portion 53 in the suction state. By such ribs 561 (suction method) as just described, even if the vaginal wall is pulled excessively in the puncture direction, the vaginal wall can be prevented or suppressed with relative certainty from being displaced in the puncture direction. Consequently, the vaginal wall during puncture can be prevented with relative certainty from being released inadvertently from the suction state.

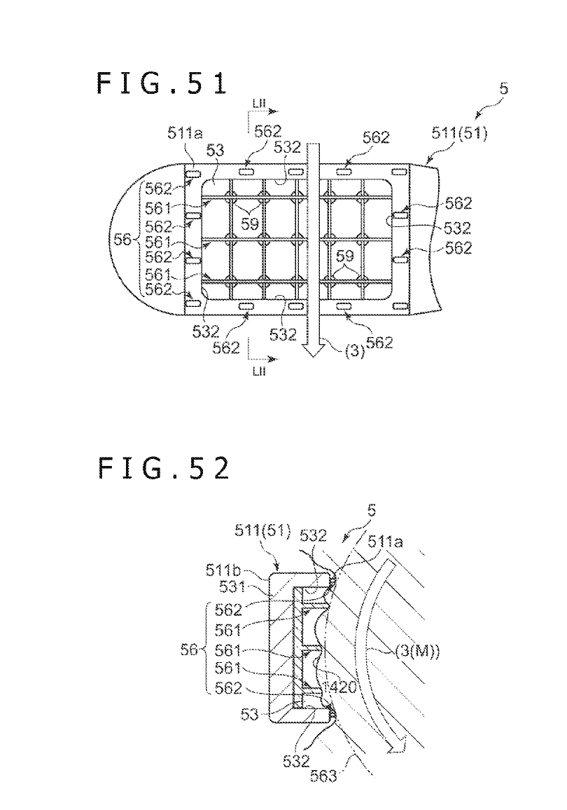

FIG. 51 is a plan view of a vaginal insertion member possessed by a living body insertion device (fourteenth embodiment). FIG. 52 is a sectional view taken along line LII-LII of FIG. 51.

In the following, the fourteenth embodiment of a living body insertion tool and a suction method of the present disclosure is described with reference to FIGS. 51 and 52. However, the description is given principally of differences of the fourteenth embodiment from the aforementioned embodiments while description of like matters are omitted herein to avoid redundancy.

The present embodiment is similar to the thirteenth embodiment described hereinabove except that it is different in configuration of the vaginal insertion member.

As depicted in FIGS. 51 and 52, in the present embodiment, the positional displacement preventing portion 56 has, in addition to the ribs 561, a plurality of small protrusions (second protrusions) 562 formed to project from the upper face 511a. The small protrusions 562 are disposed so as to surround the recessed portion 53. Consequently, the vaginal insertion member 5 is in a state in which totaling three or more of ribs 561 and small protrusions 562 are disposed.

Although the small protrusions 562 are, for example, equal in length, width and height, they are smaller than those of the ribs 561. Note that the rib 561 positioned centrally among the three ribs 561 can be lower than the ribs 561 on the opposite sides thereof.

As depicted in FIG. 52, in the positional displacement preventing portion 56, an imaginary line 563 interconnecting the tops of the three or more ribs 561 and small protrusions 562 along the puncture direction extends along a curved shape which is curved arcuately similarly to the puncture route M of the puncture member 3.

While the puncture member 3 is performing puncture, the vaginal wall is pulled excessively in the puncture direction as aforementioned, and the vaginal wall is pressed also against the vaginal insertion member 5. Since the ribs 561 and the small protrusions 562 are disposed so that the imaginary line 563 extends along an arc, the pressing force against the vaginal wall is uniform, which is desirable.

Note that, although the formed number of small protrusions 562 is, in the present embodiment, a plural number, the number is not limited to this and may be, for example, one.

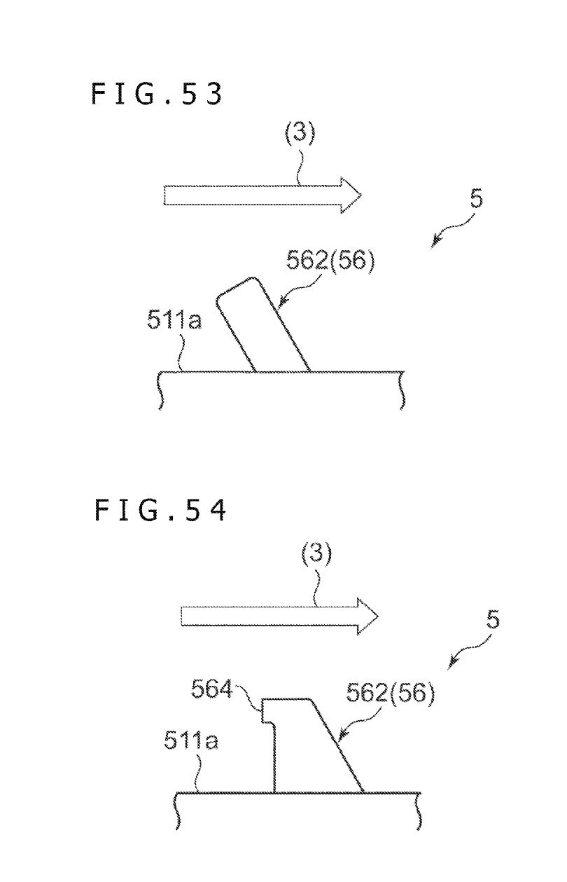

FIG. 53 is an enlarged view of a protrusion of a vaginal insertion member possessed by a living body insertion device (fifteenth embodiment).

In the following, the fifteenth embodiment of a living body insertion tool and a suction method of the present disclosure is described with reference to FIG. 53. However, the description is given principally of differences of the fifteenth embodiment from the aforementioned embodiments while description of like matters are omitted herein to avoid redundancy.

The present embodiment is similar to the thirteenth embodiment described hereinabove except that it is different in configuration of the vaginal insertion member.

As depicted in FIG. 53, in the present embodiment, the small protrusions 562 are inclined toward the puncture direction. Consequently, when the suction target portion 1420 (vaginal wall) is pulled in the puncture direction in the suction state, the small protrusions 562 sink with certainty into the suction target portion 1420. Therefore, the suction target portion 1420 can be prevented with a relative higher degree of certainty from being inadvertently released from the suction state thereof.

FIG. 54 is an enlarged view of a protrusion of a vaginal insertion member possessed by a living body insertion device (sixteenth embodiment).

In the following, the sixteenth embodiment of a living body insertion tool and a suction method of the present disclosure is described with reference to FIG. 54. However, the description is given principally of differences of the sixteenth embodiment from the aforementioned embodiments while description of like matters are omitted herein to avoid redundancy.

The present embodiment is similar to the thirteenth embodiment described hereinabove except that it is different in configuration of the vaginal insertion member.

As depicted in FIG. 54, in the present embodiment, each small protrusion 562 has a pawl 564 formed thereon so as to project toward the puncture direction. Consequently, when the suction target portion 1420 is pulled in the puncture direction in the suction state, the pawl 564 of the small protrusion 562 sinks with relative certainty into the suction target portion 1420 to prevent movement of the suction target portion 1420. By this, the suction target portion 1420 can be prevented with a higher degree of certainty from being released inadvertently from the suction state thereof.

Note that the thickness of the small protrusion 562 preferably increases toward the root side thereof, namely, toward the upper face 511a side.

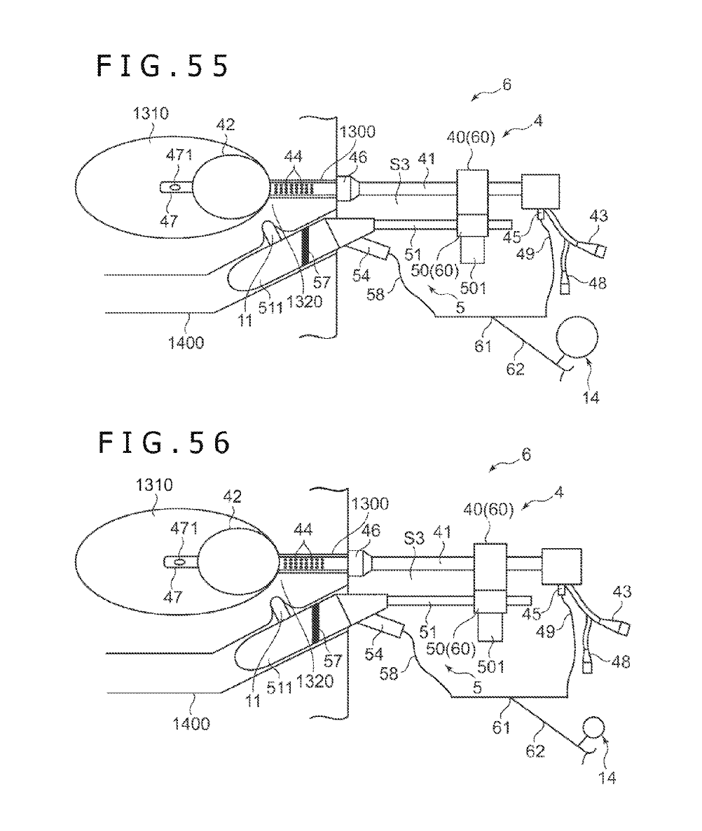

FIGS. 55 and 56 are views illustrating an operation procedure of a living body insertion device (seventeenth embodiment).

In the following, the seventeenth embodiment of a living body insertion tool and a suction method of the present disclosure is described with reference to FIGS. 55 and 56. However, the description is given principally of differences of the seventeenth embodiment from the aforementioned embodiments while description of like matters are omitted herein to avoid redundancy.

The present embodiment is similar to the first embodiment described hereinabove except that it is different in configuration of the living body insertion device.

As depicted in FIGS. 55 and 56, in the present embodiment, the suction line 49 of the urethral insertion member 4 (one of the living body insertion tools) and the suction line 58 of the vaginal insertion member 5 (the other living body insertion tool) intermediately join together at a merging location 61. A notification portion configured from an expandable balloon 14 is disposed on a line 62 on the downstream side with respect to the merging location 61. When the balloon 14 is expanded or contracted, this can be regarded as notification of at least one of the following two notifications.

The first notification relates to whether or not, when the urethral insertion member 4 sucks the urethral wall (living body tissue), the suction demonstrates a predetermined suction state, in particular, whether or not the suction is successful.

The second notification relates to whether or not, when the vaginal insertion member 5 sucks the vaginal wall (living body tissue), the suction demonstrates a predetermined suction state, namely, whether or not the suction is successful.

In accordance with an exemplary embodiment, the balloon 14 is configured such that an expandable elastic body having a form of a film is formed as a bag whose internal space is communicated with the line 62. Note that the constituent material of the elastic film is not limited particularly, and various rubber materials such as, for example, silicone rubber, can be used.

Such a balloon 14 as just described is, in the mounted state of the puncture apparatus 1, exposed from the living body. Consequently, the balloon 14 can be visually recognized with relative certainty.

Preferably, for example, a cock valve for establishing or canceling communication between the balloon 14 and the line 62 is provided at a boundary portion between the balloon 14 and the line 62. When suction is performed by any of the insertion members, the cock valve is operated to place the balloon 14 and the line 62 into a communication state.

As depicted in FIG. 55, in the initial state, namely, in the state in which suction by the urethral insertion member 4 and the vaginal insertion member 5 is not performed as yet, the balloon 14 expands itself by the elastic force thereof.

Then, if suction by the urethral insertion member 4 and the vaginal insertion member 5 is started and the suction of any of them results in success as depicted in FIG. 56, namely, if the urethral wall is sucked to the urethral insertion member 4 and/or the vaginal wall is sucked to the vaginal insertion member 5, also the inside of the balloon 14 is sucked through the line 62. Therefore, it can be recognized that the balloon 14 contracts to decrease the size thereof significantly.

In contrast, if the suction by one or both of the urethral insertion member 4 and the vaginal insertion member 5 results in failure, then the balloon 14 demonstrates little variation or demonstrates a little variation and becomes smaller. Then, which one of the urethral insertion member 4 and the vaginal insertion member 5 fails in suction can be confirmed, for example, by mounting a clamp on the suction line 49 or the suction line 58 on the upstream side with respect to the merging location 61 to perform confirmation. As an example, if the suction by the urethral insertion member 4 results in success while the suction by the vaginal insertion member 5 results in failure, then even if a clamp is mounted on the suction line 49, little variation can be observed on the balloon 14. However, if a clamp is mounted on the suction line 58, then the balloon 14 contracts. From this, it can be known that the suction by the urethral insertion member 4 results in success and the suction by the vaginal insertion member 5 results in failure. Note that a three-way stopcock may be provided at the merging location 61 in place of the clamp so that the merging location 61 serves as an openable and closable element.

Since the balloon 14 has a function for providing a notification of whether or not suction results in success in this manner, when suction of a living body is performed, only if the state of the balloon 14 is visually recognized, then success or failure in suction can be recognized readily and with relative certainty.

With such a configuration as described above, before puncture (puncture method) by the puncture member 3 is performed, while the living body tissue between the urethra 1300 and the vagina 1400 is sucked in an expansion direction, namely, in a direction in which the urethra 1300 and the vagina 1400 are spaced apart from each other, it can be confirmed whether the suction results in success or failure to perform puncture.

Preferably, the line 62 on which the balloon 14 is provided is configured from a tube having flexibility. This makes it possible to move the balloon 14 to a position at which it can be visually recognized or to a position at which the balloon 14 does not make an obstacle to the manipulation.

Further, although the balloon 14 is contracted by suction, the balloon 14 is not limited to this, and the balloon 14 may be expanded or may be deformed in some other form.

Note that, although the notification portion is configured, in the present embodiment, from the balloon 14 in the form of a bag, the notification portion is not limited to this and may be formed, for example, from a frame member to which an elastic film is applied. Further, as the notification form, notification by voice, notification by light emission or notification by a pressure gauge which issues a notification of a variation of an internal pressure may be applied.

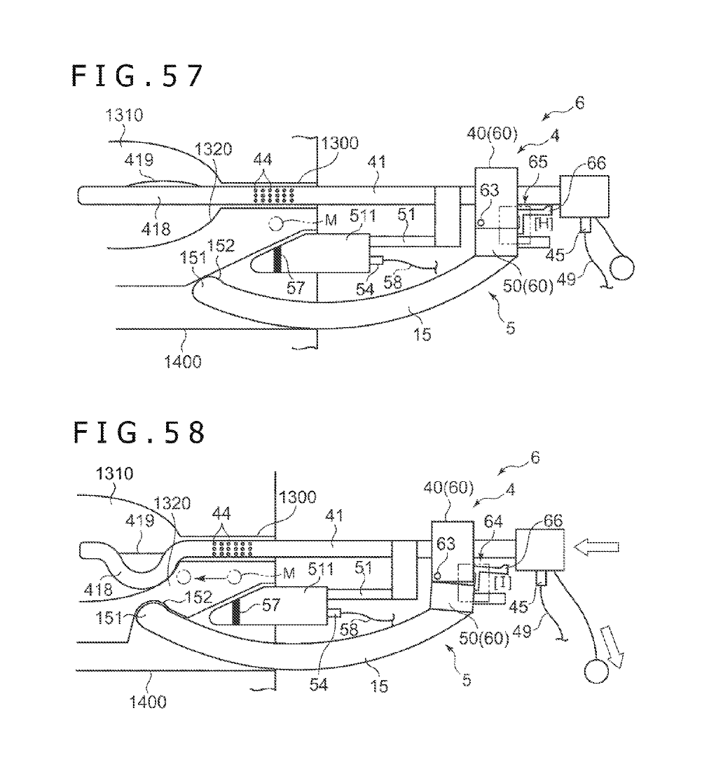

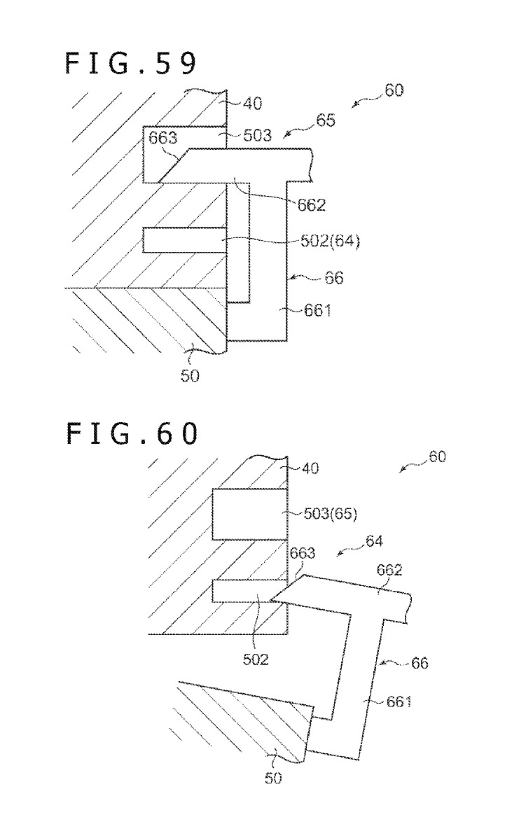

FIGS. 57 and 58 are views illustrating an operation procedure of a living body insertion device (eighteenth embodiment). FIG. 59 is an enlarged sectional view of a region [H] surrounded by a two-dot chain line in FIG. 57. FIG. 60 is an enlarged sectional view of a region [I] surrounded by a two-dot chain line in FIG. 58.

In the following, the eighteenth embodiment of a living body insertion tool and a suction method of the present disclosure is described with reference to FIGS. 57 to 60. However, the description is given principally of differences of the eighteenth embodiment from the aforementioned embodiments while description of like matters are omitted herein to avoid redundancy.

The present embodiment is similar to the first embodiment described hereinabove except that it is different in configuration of the living body insertion device.

As depicted in FIGS. 57 and 58, in the present embodiment, the support unit 60 (interlock unit) of the insertion tool 6 is configured for interlock of the urethral insertion member 4 and the vaginal insertion member 5 for movement toward and away from each other. In particular, the support unit 60 has the support portion 40 on the urethral insertion member 4 side and the support portion 50 on the vaginal insertion member 5 side as aforementioned. The support unit 60 further has a pivotally support portion 63 for supporting the support portion 40 and the support portion 50 for pivotal motion thereon. The configuration of the pivotally support portion 63 is not limited particularly and can be formed, for example, as an element configured from a shaft and a bearing. If one of the urethral insertion member 4 and the vaginal insertion member 5 pivotally moves in a predetermined direction with respect to the other through the pivotally support portion 63, then the urethral insertion member 4 and the vaginal insertion member 5 can be placed into a spaced apart state depicted in FIG. 57 in which they are spaced apart from each other. However, if one of the urethral insertion member 4 and the vaginal insertion member 5 pivotally moves in the opposite direction, then the urethral insertion member 4 and the vaginal insertion member 5 are placed into a closely positioned state depicted in FIG. 58 in which they are positioned closely to each other.

Further, as depicted in FIGS. 59 and 60, the support unit 60 has a first locking portion 64 for maintaining the closely positioned state and a second locking portion 65 for maintaining the spaced state. The first locking portion 64 has a first engaging portion 502 configured from a recess open to a proximal end face of the support portion 40. The second locking portion 65 has a second engaging portion 503 configured from a recess open to the proximal end face of the support portion 40 at a position different from that of the first engaging portion 502.

Further, the first locking portion 64 and the second locking portion 65 have a common single engaging member 66. The engaging member 66 is configured from an arm portion 661 supported on and fixed to the support portion 50 and an engaging piece 662 provided at an end portion of the arm portion 661. The engaging piece 662 can be engaged with the first engaging portion 502 and the second engaging portion 503 at timings different from each other. As depicted in FIG. 60, in the closely positioned state, the engaging piece 662 can engage with the first engaging portion 502, and the closely positioned state can be maintained with relative certainty by the engagement. As depicted in FIG. 59, in the spaced state, the engaging piece 662 can engage with the second engaging portion 503, and the spaced apart state can be maintained with relative certainty by the engagement.

Note that an inclined face 663 is formed at a distal end portion of the engaging piece 662. By the inclined face 663, getting over of the engaging piece 662 from the first engaging portion 502 to the second engaging portion 503 upon transition from the closely positioned state to the spaced apart state can be readily performed.

As depicted in FIGS. 57 and 58, in the present embodiment, the vaginal insertion portion 51 of the vaginal insertion member 5 is not supported on the support portion 50 but is supported at an intermediate portion of the urethral insertion portion 41 of the urethral insertion member 4 in the longitudinal direction. The element which is supported by the support portion 50 in place of the vaginal insertion portion 51 is an auxiliary vaginal insertion portion 15 which is provided on the vaginal insertion member 5 and configured to be inserted into the vagina 1400 together with the vaginal insertion portion 51. The auxiliary vaginal insertion portion 15 has an elongate shape and forms a curved portion, which is curved in an arch as a whole in a direction in which it is spaced apart from the urethral insertion member 4, namely, in such a manner as to stride over the vaginal insertion portion 51. Further, the auxiliary vaginal insertion portion 15 is longer than the vaginal insertion portion 51, and a distal end portion 151 of the auxiliary vaginal insertion portion 15 is positioned on the distal side with respect to the insertion portion 511 of the vaginal insertion portion 51. The distal end portion 151 has a rounded protrusion 152 formed thereon so as to project toward the urethral insertion member 4 side. The protrusion 152 can press, in the closely positioned state, a region just below the bladder 1310. Consequently, sandwiching of a living body tissue hereinafter described can be performed with relative certainty.

Further, in the present embodiment, the urethral insertion member 4 does not include the balloon 42 and can include, in place of the balloon 42, a deformable portion 418 provided at a distal end portion of the urethral insertion portion 41 and capable of deforming in a curve and an operation wire 419 for operating the deformable portion 418 to deform. The deformable portion 418 demonstrates, in a natural state thereof in which no external force is applied thereto, a linear state, but demonstrates, in a deformed state, a curved shape in which it is curved to the vaginal insertion member 5 side. In accordance with an exemplary embodiment, the operation wire 419 can be fixed at a distal end portion thereof to a distal end portion of the deformable portion 418, and a proximal end portion of the operation wire 419 can be grasped and pulled in a direction toward the proximal end. By the pulling, the deformable portion 418 is deformed into a curved state and therefore is attached in the deformed state to the bladder neck 1320.

In the insertion tool 6 having such a configuration as described above, the urethral insertion member 4 and the vaginal insertion member 5 are first placed into the spaced state and mounted on a living body as depicted in FIG. 57. Note that, as aforementioned, the spaced apart state is maintained with relative certainty by the second locking portion 65. This makes it possible to establish the mounted state on the living body.

Then, the urethral insertion member 4 and the vaginal insertion member 5 are placed into the closely positioned state as depicted in FIG. 58. In accordance with an exemplary embodiment, this closely positioned state can be maintained with relative certainty by the first locking portion 64 as aforementioned. Further, in the closely positioned state, the living body tissue between the urethra 1300 and the vagina 1400 can be sandwiched between them, and if the urethral insertion member 4 and the vaginal insertion member 5 are pushed in as they are toward the distal end, then the sandwiched living body tissue can be moved in the direction as well. Consequently, the position at which the puncture route M is to be formed can be adjusted.

In this manner, before puncture (puncture method) by the puncture member 3 is performed, the living body tissue between the urethra 1300 and the vagina 1400 can be moved forwardly or rearwardly thereby to adjust the position of the living body tissue. After the adjustment, a puncture process, namely, a surgical treatment for the living body tissue, can be performed relative accurately and with relative certainty.

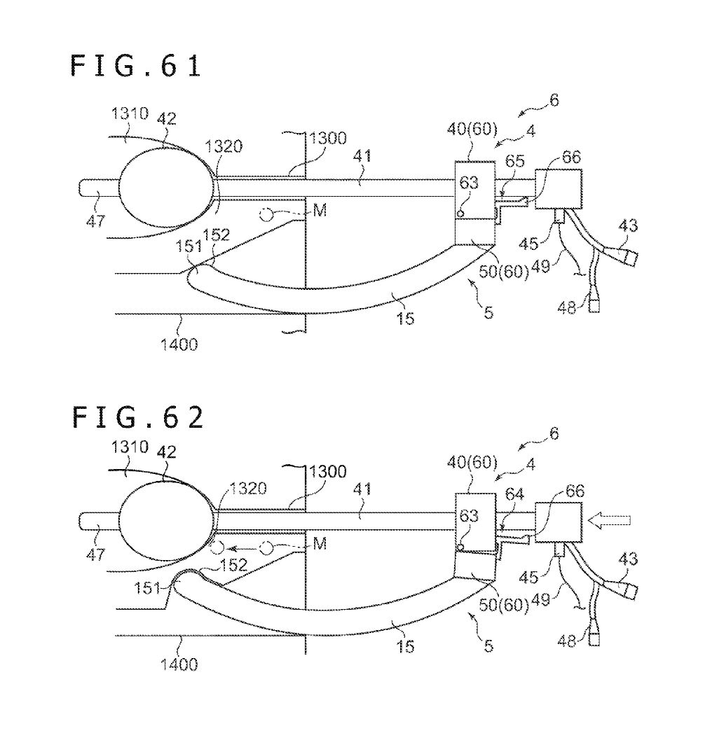

FIGS. 61 and 62 are views illustrating an operation procedure of a living body insertion device (nineteenth embodiment).

In the following, the nineteenth embodiment of a living body insertion tool and a suction method of the present disclosure is described with reference to FIGS. 61 and 62. However, the description is given principally of differences of the nineteenth embodiment from the aforementioned embodiments while description of like matters are omitted herein to avoid redundancy.

The present embodiment is similar to the eighteenth embodiment described hereinabove except that it is different in configuration of the living body insertion device.

As depicted in FIGS. 61 and 62, in the present exemplary embodiment, the vaginal insertion member 5 is configured such that it does not include the vaginal insertion portion 51 but has the auxiliary vaginal insertion portion 15. In this case, the auxiliary vaginal insertion portion 15 serves as a main body portion of the vaginal insertion member 5, and the vaginal insertion member 5 can be formed in a simplified structure.

Further, since the vaginal insertion portion 51 is omitted, when the auxiliary vaginal insertion portion 15 is inserted into the vagina 1400 as depicted in FIG. 61, the state of the vaginal wall can be readily confirmed.

Further, as depicted in FIG. 62, when the living body tissue is sandwiched in the closely positioned state, it can be confirmed to which degree the auxiliary vaginal insertion portion 15 is inserted into the vagina 1400 and at which position the living body tissue is sandwiched.

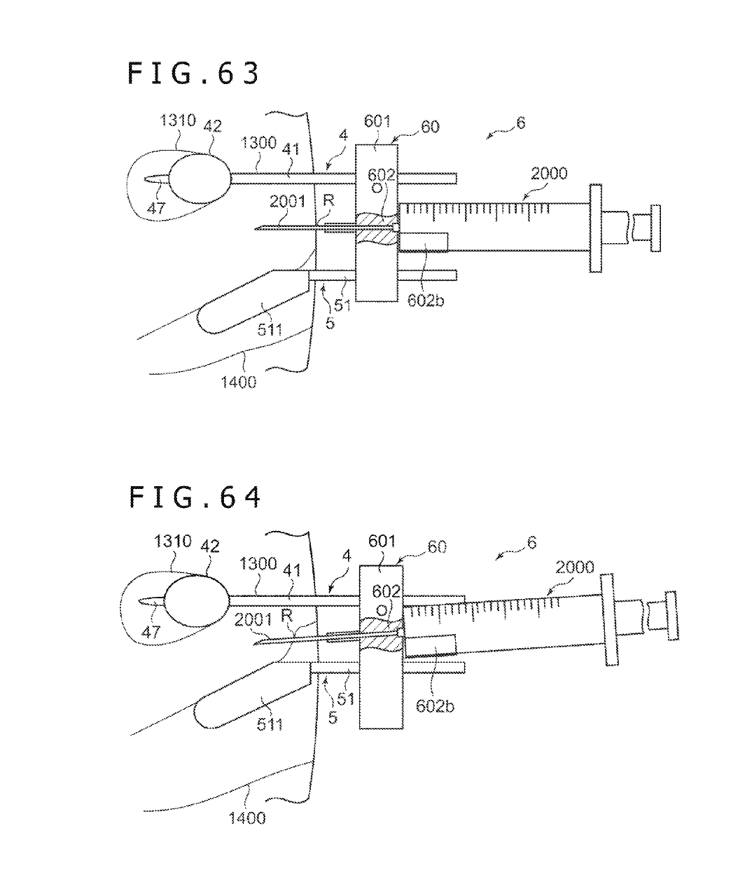

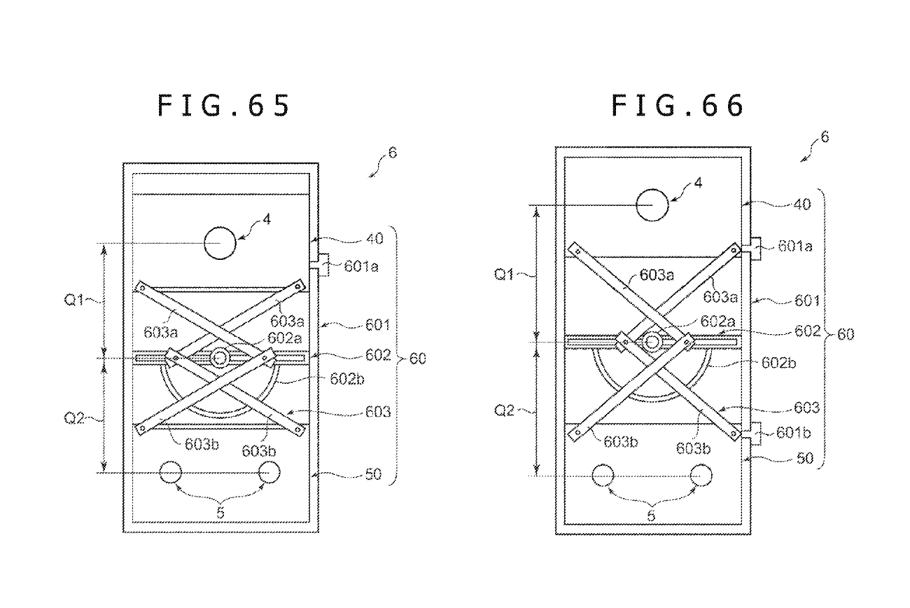

FIGS. 63 and 64 are views illustrating an operation procedure of a living body insertion device (twentieth embodiment). FIG. 65 is a front elevational view of a support unit of the living body insertion device (twentieth embodiment).

In the following, the twentieth embodiment of a living body insertion tool and a suction method of the present disclosure is described with reference to FIGS. 63 to 65. However, the description is given principally of differences of the twentieth embodiment from the aforementioned embodiments while description of like matters are omitted herein to avoid redundancy.

The present embodiment is similar to the first embodiment described hereinabove except that it is different in configuration of the living body insertion device.

As depicted in FIGS. 63 and 64, in the present embodiment, the support unit 60 is configured such that it supports the urethral insertion member 4 and the vaginal insertion member 5 for movement toward and away from each other. In particular, for example, as depicted in FIG. 65, the support unit 60 can include a frame member 601, a connection portion 602 and a position restriction portion 603 in addition to the support portions 40 and 50.

The frame member 601 supports, on the inner side thereof, the support portion 40, connection portion 602 and support portion 50 in order from above in FIG. 65. The support portion 40 and the connection portion 602 are supported for movement in the upward and downward direction in FIG. 65 with respect to the frame member 601. The mechanism for the movement is not limited particularly and can be configured, for example, from a groove and a protrusion inserted in the groove and movable along the groove. The support portion 50 is supported fixedly on the frame member 601.

The connection portion 602 has a form of a plate and has, at a central portion in the longitudinal direction thereof, a connection opening 602a to which the injector 2000 is to be connected. As depicted in FIG. 64, in the state in which the injector 2000 is connected to the connection opening 602a, the injection needle 2001 mounted on the injector 2000 projects in a direction toward the distal end. Further, the connection portion 602 has an arcuate supporting plate 602b which supports the injector 2000 from the vaginal insertion member 5 side in the state in which the injector 2000 is connected thereto. The injector 2000 can be stably connected to the connection portion 602 by the supporting plate 602b.

The position restriction portion 603 is a link mechanism configured from two first links 603a and two second links 603b. The first links 603a cross with each other and interlock the support portion 40 and the connection portion 602 to each other. The first links 603a are each supported at a pivotal support point on the support portion 40 and the connection portion 602. Meanwhile, the second links 603b cross with each other and interlock the connection portion 602 and the support portion 50 to each other. The second links 603b are each supported at a pivotal support point on the connection portion 602 and the support portion 50. By the position restriction portion 603 having such a configuration as just described, the support portion 40 and the connection portion 602 can move in the same direction in an interlocking relationship with the support portion 50, namely, move toward and away from the support portion 50. Consequently, as depicted in FIG. 65, for example, irrespective of whether the support portion 40 and the connection portion 602 move toward or away from the support portion 50 in the state in which the connection portion 602 and the injector 2000 are connected to each other, the ratio between the distance Q1 between the injection needle 2001 and the urethral insertion member 4 and the distance Q2 between the injection needle 2001 and the vaginal insertion member 5 is fixed. The position restriction portion 603 performs such restriction that the support portion 40 (urethral insertion member 4), the support portion 50 (vaginal insertion member 5) and the connection portion 602 maintain such a positional relationship as described just above.

Note that the frame member 601 can include a locking member 601a for temporarily restricting, when the support portion 40 and the connection portion 602 move in an interlocking relationship with each other, re-movement at the destination of the movement. The locking member 601a is engageable with the support portion 40, and this engagement restricts re-movement of the support portion 40 and the connection portion 602. Then, if the engagement is cancelled, then such re-movement is permitted.

As depicted in FIGS. 63 and 64, the thickness of the living body tissue between the urethra 1300 and the vagina 1400 has an individual difference and may be comparatively large (refer to FIG. 63) or comparatively small (refer to FIG. 64).

On a patient who has the living body tissue of a comparatively large thickness as depicted in FIG. 63, the urethral insertion member 4 and the vaginal insertion member 5 are mounted in a state spaced apart from each other. On the other hand, on a patient who has the living body tissue of a comparatively small thickness as depicted in FIG. 64, the urethral insertion member 4 and the vaginal insertion member 5 are mounted in a closely positioned relationship with each other. In any one of the state depicted in FIG. 63 and the state depicted in FIG. 64, the ratio between the distance Q1 between the injection needle 2001 and the urethral insertion member 4 and the distance Q2 between the injection needle 2001 and the vaginal insertion member 5 is fixed with certainty by the action of the position restriction portion 603 as aforementioned. Consequently, when a living body tissue is punctured by the injection needle 2001, the puncture location R can be fixed irrespective of the individual difference, namely, can be set to a substantially central portion of the living body tissue in the thicknesswise direction. If the living body tissue is punctured in this state and liquid is injected to perform liquid dissection (injection method), then also in this case, the living body tissue can be inflated rapidly to a state in which the living body tissue is sucked to the urethral insertion member 4 and the vaginal insertion member 5 irrespective of the individual difference. In addition, the puncture location R can be set to a position displaced from a region of the living body tissue which is actually sucked by the vaginal insertion member 5 (refer to FIG. 64). Consequently, the liquid used for the liquid dissection can be prevented from being sucked from the living body tissue by the vaginal insertion member 5.

Further, as aforementioned, the living body tissue is inflated by liquid dissection. As depicted in FIG. 64, in the insertion tool 6, preferably the connection portion 602 is configured for rotation with respect to the frame member 601. By the rotation, the injection needle 2001 can follow up a variation of the living body tissue being inflated. Consequently, excessive force can be prevented from acting upon the living body tissue from the injection needle 2001.

FIG. 66 is a front elevational view of a support unit of a living body insertion device (twenty-first embodiment).

In the following, the twenty-first embodiment of a living body insertion tool and a suction method of the present disclosure is described with reference to FIG. 66. However, the description is given principally of differences of the twenty-first embodiment from the aforementioned embodiments while description of like matters are omitted herein to avoid redundancy.

The present embodiment is similar to the twentieth embodiment described hereinabove except that it is different in configuration of the living body insertion device.

As depicted in FIG. 66, in the support unit 60 in the present embodiment, the support portion 40 and the support portion 50 are supported on the frame member 601 for movement in the upward and downward direction in FIG. 66. The connection portion 602 is supported fixedly on the frame member 601.

The support portion 40 and the support portion 50 can move in an interlocking relationship in the opposite directions to each other with respect to the connection portion 602 by the position restriction portion 603, namely, can move toward and away from each other. Consequently, in the closely positioned state and the spaced apart state of the support portion 40 and the support portion 50, the ratio between the distance Q1 between the injection needle 2001 (connection portion 602) and the urethral insertion member 4 and the distance Q2 between the injection needle 2001 (connection portion 602) and the vaginal insertion member 5 is fixed. Therefore, when the living body tissue is punctured by the injection needle 2001, the puncture location R can be fixed irrespective of the individual difference.

Note that the frame member 601 can include a locking member 601a and another locking member 601b each of which temporarily restricts, when the support portion 40 and the support portion 50 are moved in an interlocking relationship with each other, re-movement at the destination of the movement. The locking member 601a is engageable with the support portion 40, and the re-movement is restricted by the engagement. The re-movement is permitted by cancelling the engagement. The locking member 601b is engageable with the support portion 50, and the re-movement is restricted by the engagement. The re-movement is permitted by cancelling the engagement.

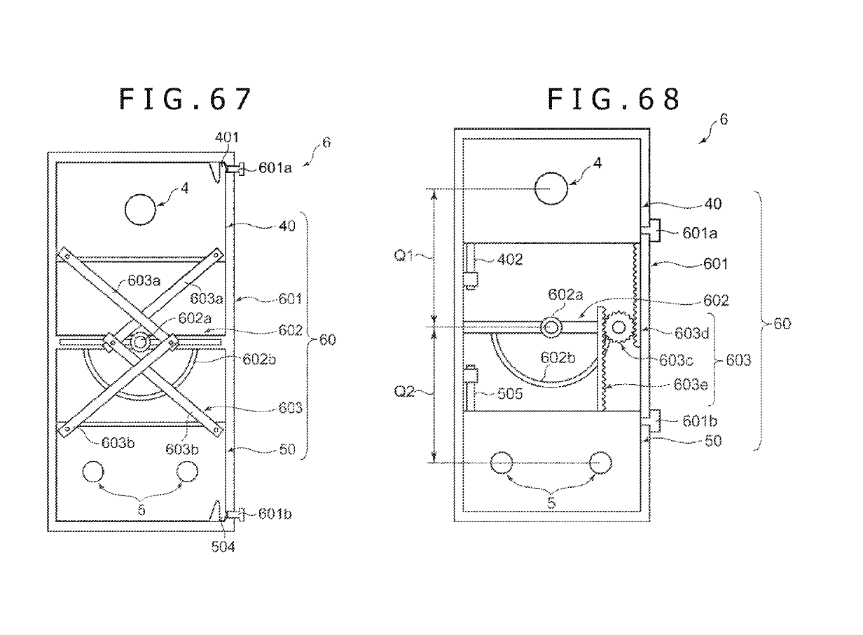

FIG. 67 is a front elevational view of a support unit of a living body insertion device (twenty-second embodiment).

In the following, the twenty-second embodiment of a living body insertion tool and a suction method of the present disclosure is described with reference to FIG. 67. However, the description is given principally of differences of the twenty-second embodiment from the aforementioned embodiments while description of like matters are omitted herein to avoid redundancy.

The present embodiment is similar to the twenty-first embodiment described hereinabove except that it is different in configuration of the living body insertion device.

As depicted in FIG. 67, in the present embodiment, the support portion 40 has an engaging portion 401 which engages with the locking member 601a while the support portion 50 has an engaging portion 504 which engages with the locking member 601b in the state in which the support portion 40 and the support portion 50 are spaced apart from each other.

The engaging portion 401 is elastically deformable and is elastically deformed by being pressed by the locking member 601a in a state in which the engaging portion 401 engages with the locking member 601a. By the elastic deformation of the engaging portion 401, the state in which the engaging portion 401 and the locking member 601a engage with each other is maintained with relative certainty. Further, by pulling the locking member 601a in this engaging state, the engagement between the engaging portion 401 and the locking member 601a can be cancelled.

Also the engaging portion 504 is elastically deformable and is elastically deformed by being pressed by the locking member 601b in a state in which the engaging portion 504 engages with the locking member 601b. By the elastic deformation of the engaging portion 504, the state in which the engaging portion 504 and the locking member 601b engage with each other is maintained with relative certainty. Further, by pulling the locking member 601b in this engaging state, the engagement between the engaging portion 504 and the locking member 601b can be cancelled.

FIG. 68 is a front elevational view of a support unit of a living body insertion device (twenty-third embodiment).

In the following, the twenty-third embodiment of a living body insertion tool and a suction method of the present disclosure is described with reference to FIG. 68. However, the description is given principally of differences of the twenty-third embodiment from the aforementioned embodiments while description of like matters are omitted herein to avoid redundancy.

The present embodiment is similar to the twenty-first embodiment described hereinabove except that it is different in configuration of the living body insertion device.

As depicted in FIG. 68, in the present embodiment, the position restriction portion 603 is configured from a pinion gear 603c supported for rotation on the connection portion 602, a rack 603d supported fixedly on the support portion 40, and a rack 603e supported fixedly on the support portion 50. The rack 603d and the rack 603e are disposed with the pinion gear 603c interposed therebetween and are held in meshing engagement with the pinion gear 603c. By the position restriction portion 603 of such a configuration as just described, the ratio between the distance Q1 between the injection needle 2001 and the urethral insertion member 4 and the distance Q2 between the injection needle 2001 and the vaginal insertion member 5 is fixed. Consequently, when the injection needle 2001 punctures a living body tissue, the puncture location R can be fixed irrespective of individual differences.

Further, a stopper 402 is provided on the support portion 40 such that it projects toward the support portion 50 side, and another stopper 505 is provided on the support portion 50 such that it projects toward the support portion 40 side. When the support portion 40 and the support portion 50 are positioned nearest to each other, the stopper 402 and the stopper 505 attach to each other, by which the shortest distance between them can be restricted.

Further, in the present embodiment, preferably the connection opening 602a is configured for sliding movement in the leftward and rightward direction in FIG. 68.

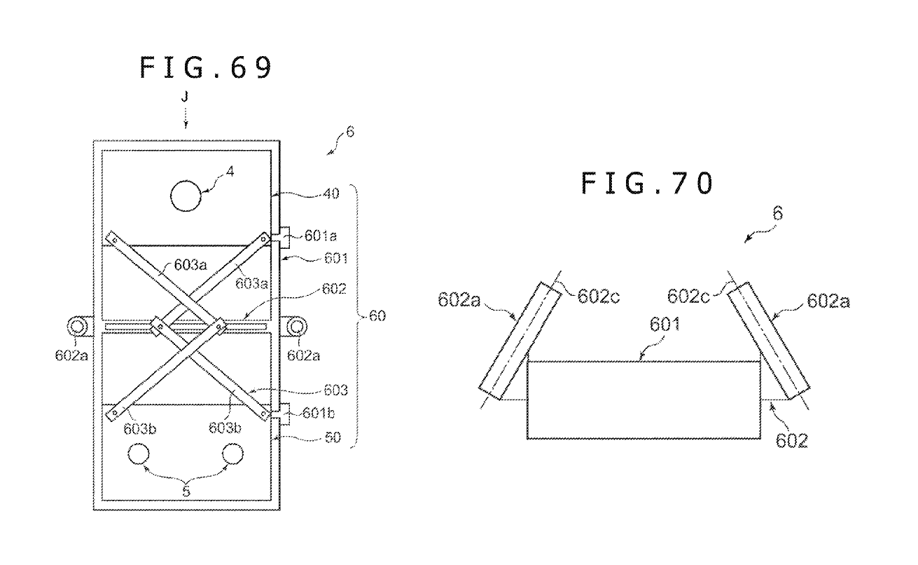

FIG. 69 is a front elevational view of a support unit of a living body insertion device (twenty-fourth embodiment). FIG. 70 is a view of the support unit of FIG. 69 as viewed in a direction indicated by an arrow mark J.

In the following, the twenty-fourth embodiment of a living body insertion tool and a suction method of the present disclosure is described with reference to FIGS. 69 and 70. However, the description is given principally of differences of the twenty-fourth embodiment from the aforementioned embodiments while description of like matters are omitted herein to avoid redundancy.

The present embodiment is similar to the twenty-first embodiment described hereinabove except that it is different in configuration of the living body insertion device.

As depicted in FIGS. 69 and 70, in the present embodiment, two connection portions 602 having a tubular shape are provided. As depicted in FIG. 70, the two connection portions 602 are disposed with the frame member 601 interposed therebetween such that the center axes 602c thereof approach each other in a direction toward the distal ends thereof. By such disposition, the connection direction of the injector 2000 is different between the connection portions 602. Consequently, if the two connection portions 602 are selectively used, then a living body tissue can be punctured from a different direction to inject liquid. Therefore, the time required for liquid dissection can be reduced.

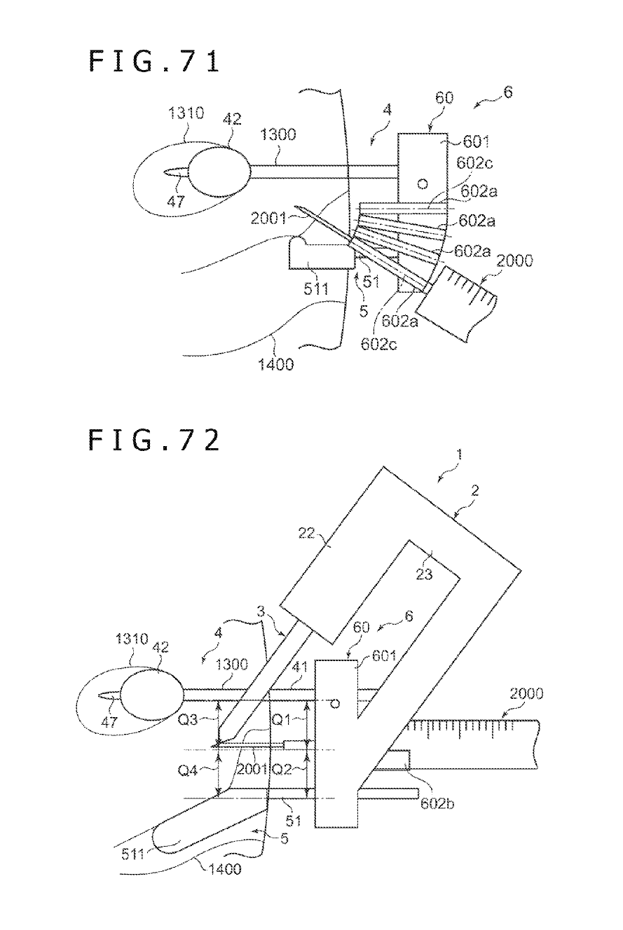

FIG. 71 is a lateral view of a living body insertion device (twenty-fifth embodiment).

In the following, the twenty-fifth embodiment of a living body insertion tool and a suction method of the present disclosure is described with reference to FIG. 71. However, the description is given principally of differences of the twenty-fifth embodiment from the aforementioned embodiments while description of like matters are omitted herein to avoid redundancy.

The present embodiment is similar to the twenty-fourth embodiment described hereinabove except that it is different in configuration of the living body insertion device.

As depicted in FIG. 71, in the present embodiment, a plurality of connection openings 602a are provided. The connection portions 602 are disposed along the longitudinal direction of the frame member 601, namely, along the upward and downward direction in FIG. 71. Further, the connection openings 602a are disposed so as to draw a circular locus centered at a predetermined position. The “predetermined position” here is a distal end arrival point of the injector 2000 and is set so as to reach a point above a projecting portion existing at the distal end of the vaginal insertion portion 51. However, the “predetermined position” is not limited to this, but may be decided from a position of a marker possessed by the urethral insertion portion 41 or the vaginal insertion portion 51. By this, the connection direction of the injector 2000 is different between the connection portions 602. Therefore, a living body tissue can be punctured with injected liquid from different directions.

In this manner, in the present embodiment, the stoppers for the support portions 40 and 50 may be the upper and lower frames of the support unit 60 or such engaging portions 504 as described in connection with the twenty-second embodiment.

FIG. 72 is a lateral view of a living body insertion device (twenty-sixth embodiment).

In the following, the twenty-sixth embodiment of a living body insertion tool and a suction method of the present disclosure is described with reference to FIG. 72. However, the description is given principally of differences of the twenty-sixth embodiment from the aforementioned embodiments while description of like matters are omitted herein to avoid redundancy.

The present embodiment is similar to the twentieth embodiment described hereinabove except that it is different in configuration of the living body insertion device.

As depicted in FIG. 72, in the present embodiment, the frame 2 and the support unit 60 are interlocked to each other, and the frame 2 in the interlocked state exhibits a function as a position restriction portion for restricting the positional relationship among the urethral insertion member 4, the vaginal insertion member 5 and the puncture member 3. In particular, when the needle body 35 (needle tip 351) passes the region between the urethral insertion member 4 and the vaginal insertion member 5, the frame 2 performs positioning such that the ratio between the distance Q3 between the needle body 35 and the urethral insertion member 4 and the distance Q4 between the needle body 35 and the vaginal insertion member 5 is fixed irrespective of whether the urethral insertion member 4 and the vaginal insertion member 5 are positioned closely or are spaced apart from each other. Consequently, puncture (puncturing method) by the puncture member 3 can be carried out relatively accurately while the puncture location of a living body tissue by the needle body 35 is fixed irrespective of individual differences. Further, for example, the ratio between the distance Q3 and the distance Q4 and the aforementioned ratio between the distance Q1 and the distance Q2 are equal to each other.

In treatment of urinary incontinence of women, the place actually punctured by the puncture member 3 and the place actually punctured by the injection needle 2001 are preferably made same as each other in the living body tissue. Therefore, the configuration wherein the aforementioned two ratios are equal to each other is effective.

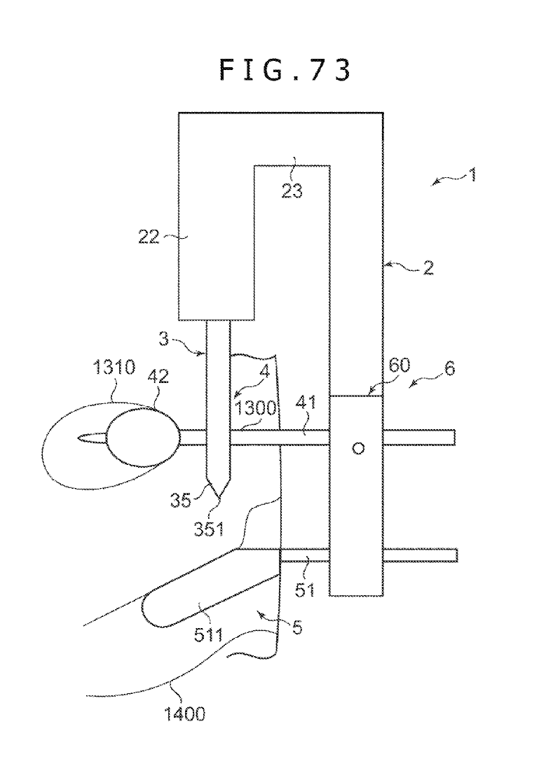

FIG. 73 is a lateral view of a living body insertion device (twenty-seventh embodiment).

In the following, the twenty-seventh embodiment of a living body insertion tool and a suction method of the present disclosure is described with reference to FIG. 73. However, the description is given principally of differences of the twenty-seventh embodiment from the aforementioned embodiments while description of like matters are omitted herein to avoid redundancy.

The present embodiment is similar to the twentieth embodiment described hereinabove except that it is different in configuration of the living body insertion device.

As depicted in FIG. 73, in the present embodiment, the connection portion 602 (connection opening 602a and supporting plate 602b) is omitted, and the support unit 60 is simplified in configuration as much.

Note that, in the present embodiment, the puncture member 3 is disposed such that a puncture hole by the puncture member 3 is formed in a direction orthogonal to the longitudinal direction of the insertion tool 6.

While the living body insertion tool and the suction method of the present disclosure have been described with reference to the embodiments depicted in the drawings, the present disclosure is not limited to them and the components which configure the living body insertion tool and the suction method can be replaced by arbitrary components which can demonstrate similar functions. Further, some other arbitrary member or members may be added to the present disclosure.

Further, the living body insertion tool and the suction method of the present disclosure may be two or more arbitrary ones of the configurations (features) of the embodiments described hereinabove.

Further, while, in the embodiments described hereinabove, the needle body is held on the main body in a freely detachable manner, the needle body is not limited to this and may be fixed on the main body such that, for example, the main body and the needle body are formed as a unitary member. In this case, by operating the puncture member to puncture a living body and project the needle body to the outside of the living body and then cutting the needle body using scissors or the like, the distal side opening of the main body can be opened.

Further, in the embodiments described hereinabove, the main body of the puncture member is disposed into a living body, whereafter the implant main body is inserted into the main body. However, the puncture member is not limited to this, and the implant main body may be accommodated in the puncture member (main body) from the beginning. In this case, it is preferable, for example, to fix a string positioned on the needle tip side from between two strings possessed by the implant main body to the needle tip. Where the string is fixed in this manner, if the needle tip is removed from the main body, then the string can be thereby projected to the outside of the main body. Therefore, later fine adjustment of the disposition of the implant main body can be smoothly performed.

Further, while, in the foregoing description of the embodiments, the puncture apparatus is applied to an apparatus for use when an implant which can be embedded for treatment of urinary incontinence of women is embedded into a living body. However, the application of the puncture apparatus is not limited to this.

For example, the target of the application of the present disclosure can include excretory disorders attendant on the weakening of the pelvic floor muscle group (urinary urgency, frequent urination, urinary incontinence, fecal incontinence, urinary retention, and strangury) and pelvic floor disorders including pelvic organ prolapse, vesicovaginal fistula, urethrovaginal fistula, or pelvic pain. The pelvic organ prolapse can include disorders of cystocele, enterocele, rectocele, and hysterocele. Alternatively, the pelvic organ prolapse can include such disorders as anterior vaginal prolapse, posterior vaginal prolapse, vaginal apical prolapse, and vaginal vault prolapse in which the naming method thereof is based on the prolapsed vaginal-wall part.

Further, the overactive tissues include the bladder, vagina, uterus, and bowel. The less active tissues include bones, muscles, fascias, and ligaments. Especially, for example, in the case of pelvic floor disorders, the less active tissues can include an obturator fascia, a coccygeus fascia, a cardinal ligament, an uterosacral ligament, and a sacrospinous ligament.

The procedure for interlocking an overactive tissue with a less active tissue in the pelvic floor disorder can include a retropubic sling surgery, a transobturator sling surgery (transobturator tape (TOT) surgery), a tension-free vaginal mesh (TVM) surgery, a uterosacral ligament suspension (USLS) surgery, a sacrospinous ligament fixation (SSLF) surgery, an iliococcygeus fascia fixation surgery, and a coccygeus fascia fixation surgery.

According to the present disclosure, the living body insertion tool for being inserted into a body lumen can include an attachment face configured to attach to a living body tissue, at least one suction portion configured to suck the living body tissue to the attachment face side in a state in which the attachment face attaches to the living body tissue, and a blocking portion configured to block the living body tissue from reaching the suction portion. Therefore, a drop of the suction force can be prevented with relative certainty.

Accordingly, the living body insertion tool of the present disclosure has industrial applicability.

The detailed description above describes to a living body insertion tool and a suction method. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A living body insertion tool for being inserted into a body lumen, comprising:
an attachment face configured to attach to a living body tissue;
a suction portion configured to suck the living body tissue to the attachment face in a state in which the attachment face attaches to the living body tissue; and
a blocking portion configured to block the living body tissue from reaching the suction portion.

2. The living body insertion tool according to claim 1, wherein a recessed portion is formed on the attachment face;
the suction portion is provided at a bottom portion of the recessed portion; and
the blocking portion is configured from a plurality of linear members at least part of which is provided at the bottom portion and which individually have a linear shape or a shape of a belt.

3. The living body insertion tool according to claim 2, wherein the blocking portion has a form of a grating formed by crossing the plurality of linear members with each other.

4. The living body insertion tool according to claim 2, wherein the blocking portion and the bottom portion have a gap therebetween and are communicated with each other by the gap.

5. The living body insertion tool according to claim 2, wherein at least part of the linear members is provided so as to connect side walls of the recessed portion to each other.

6. The living body insertion tool according to claim 2, wherein the linear members are disposed so as to overlap with a suction direction of the suction portion.

7. The living body insertion tool according to claim 1, wherein the body lumen is a vaginal cavity and the living body insertion tool is inserted into and used with the vaginal cavity.

8. A suction method for inserting a living body insertion tool into a body lumen to suck a living body tissue to the living body insertion tool side, the living body insertion tool including an attachment face configured to attach to the living body tissue, a suction portion configured to suck the living body tissue to the attachment face in a state in which the attachment face attaches to the living body tissue, and a blocking portion configured to block the living body tissue from reaching the suction portion, the suction method comprising:
sucking the living body tissue to a recessed portion of the attachment face such that the living body tissue does not attach to the suction portion.

9. The method according to claim 8, comprising:
forming a recessed portion on the attachment face;
providing the suction portion at a bottom portion of the recessed portion; and
configuring the blocking portion from a plurality of linear members at least part of which is provided at the bottom portion and which individually have a linear shape or a shape of a belt.

10. The method according to claim 9, wherein the blocking portion has a form of a grating formed by crossing the plurality of linear members with each other.

11. The method according to claim 9, wherein the blocking portion and the bottom portion have a gap therebetween and are communicated with each other by the gap.

12. The method according to claim 9, comprising:
providing at least part of the linear members so as to connect side walls of the recessed portion to each other.

13. The method according to claim 9, comprising:
disposing the linear members so as to overlap with a suction direction of the suction portion.

14. The method according to claim 8, wherein the body lumen is a vaginal cavity; and
inserting the living body insertion tool into the vaginal cavity.
